(19) United States
(12) Patent Application Publication
(10) Pub. No.: US 2026/0325489 A1
(43) Pub. Date: Oct. 22, 2026

(54) PHARMACEUTICAL COMPOSITIONS COMPRISING 5-METHOXY-N,N-DIMETHYLTRYPTAMINE (5-MEO-DMT)

(71) Applicant: Beckley Psytech Limited, Oxford (GB)

(72) Inventor: Jason GRAY, Oxford (GB)

(21) Appl. No.: 19/492,890

(22) PCT Filed: Jun. 13, 2024
(86) PCT No.: PCT/GB2024/051512

§ 371 Date: Dec. 12, 2025

Related U.S. Application Data
(60) Provisional application No. 18/229,041, filed on Aug. 1, 2023.

(30) Foreign Application Priority Data
Aug. 1, 2023 (GB) 2308830.5
Sep. 29, 2023 (GB) 2314993.3
Oct. 3, 2023 (GB) 2315176.4
Nov. 3, 2023 (GB) 2316902.2

(51) Int. Cl. A61K 31/4045; A61K 9/00; A61K 9/16; A61K 9/19; A61K 47/12; A61K 47/32; A61M 16/12

(52) U.S. Cl. CPC A61K 31/4045; A61K 9/006; A61K 9/0075; A61K 9/0078; A61K 9/1623; A61K 9/1694; A61K 9/19; A61K 47/12; A61K 47/32; A61M 16/12

(57) ABSTRACT

This invention relates to a nasal delivery device comprising 5-methoxy-N,N-dimethyltryptamine (5-MeO-DMT) pharmaceutical compositions or formulations, more particularly intranasal pharmaceutical compositions or formulations of pharmaceutically acceptable salts of 5-MeO-DMT, and methods of administration and treatment using the same.

### FIELD OF THE INVENTION

This invention relates to an active nasal delivery device comprising 5-methoxy-N,N-dimethyltryptamine (5-MeO-DMT) pharmaceutical compositions or formulations, more particularly intranasal pharmaceutical compositions or formulations of pharmaceutically acceptable salts of 5-MeO-DMT, and methods of administration and treatment using the same.

### BACKGROUND OF THE INVENTION

5-methoxy-N,N-dimethyltryptamine is a pharmacologically active compound of the tryptamine class and has the chemical formula:

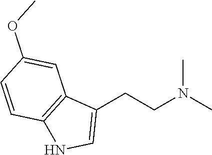 [SMILES: COc1ccc2[nH]cc(CCN(C)C)c2c1]

5-MeO-DMT is a psychoactive/psychedelic substance found in nature. Man-made salts of 5-MeO-DMT are also known in the art e.g. Sherwood, Alexander M., et al. “Synthesis and Characterization of 5-MeO-DMT succinate for clinical use.” ACS omega 5.49 (2020): 32067-32075 discloses the hydrochloride salt of 5-MeO-DMT. However, 5-MeO-DMT, and salts thereof, are not well understood and methods of administration, in particular intranasal methods of administration of pharmaceutical compositions or formulations of this compound, and the salts thereof, are difficult to develop and have not been well explored.

For example, it has been found that liquid intranasal pharmaceutical compositions or formulations of the hydrochloride salt of 5-MeO-DMT experience issues relating to stability, discoloration and a reduction in desirable pharmacokinetic properties. For the sake of brevity, the term ‘5-MeO-DMT’ used herein, may also be understood to be referring to the salts of 5-MeO-DMT.

5-MeO-DMT is not suitable for oral delivery and so other methods of administration have been considered. Other methods of administration are possible e.g. intravenous and inhalation by smoking. Intranasal administration, e.g. nasal liquid spray formulation, is also another way of providing systemic drug delivery across the blood brain barrier, in particular when oral administration is not effective.

There remains a need in the art for improved pharmaceutical compositions or formulations, in particular intranasal pharmaceutical compositions or formulations, comprising 5-MeO-DMT and the salts thereof, and methods of administration and treatment using the same to enable safe and non-invasive dosing that allows for greater accessibility for patients and greater patient compliance.

### SUMMARY OF THE INVENTION

The nasal cavity is recognised as a promising systemic drug delivery route due to the highly vascularised capillary bed within the nasal mucosa. The inventors have surprisingly discovered, see Example 28, that delivery of 5-MeO-DMT pharmaceutical compositions or formulations by an active nasal delivery device produces a desirable nasal deposition profile whilst delivery of 5-MeO-DMT pharmaceutical compositions or formulations by a passive nasal delivery device produces an undesirable nasal deposition profile.

Herein disclosed, there is provided an active nasal delivery device comprising a pharmaceutical composition/formulation of 5-MeO-DMT, or a pharmaceutically acceptable salt, prodrug, hydrate, ester, co-crystal or deuterated form thereof, and one or more pharmaceutically acceptable carriers or excipients. In an embodiment, the pharmaceutical composition/formulation of 5-MeO-DMT is a spray dried formulation. In an embodiment, the pharmaceutical composition/formulation comprises below about 5% moisture content by weight of the formulation.

In an embodiment, at least 95% of the particles of the pharmaceutical composition/formulation are larger than 10 microns in size. In an embodiment, the pharmaceutical composition/formulation comprises methyl cellulose, optionally a high viscosity methyl cellulose. In an embodiment, the pharmaceutical composition/formulation comprises a low viscosity methyl cellulose, and a high viscosity methyl cellulose. In an embodiment, the pharmaceutical composition/formulation comprises a cellulose like/based excipient; optionally HPMC, further optionally a high viscosity HPMC. In an embodiment, the pharmaceutical composition/formulation comprises a low viscosity HPMC and a high viscosity HPMC.

In an embodiment, the ratio of the low viscosity HPMC to high viscosity HPMC is in the ratio of 1:10 to 10:1, optionally 1:4 to 4:1 and further optionally 1:2 to 2:1. In an embodiment, the ratio of the low viscosity HPMC to high viscosity HPMC is in the ratio of 50:50, 45:55, 40:60, 35:65, 30:70 or 25:75. In an embodiment, the high viscosity HPMC has a viscosity greater or equal to about 20, 30, 40, 50 or 60 megaPascals, optionally where the HPMC is a HPMC containing about 7.0-12.0% hydroxypropyl content, about 28.0-30.0% methoxy content, and a viscosity of about 50 mPas. In an embodiment, the low viscosity HPMC has a viscosity less than about 20, 15, 10, 5, 1 megaPascals, optionally where the HPMC is a HPMC containing about 7.0-12.0% hydroxypropyl content, about 28.0-30.0% methoxy content, and a viscosity of about 4.8-7.2 mPas.

In an embodiment, the pharmaceutical composition/formulation comprises a polyol, optionally the polyol is mannitol, xylitol, sorbitol, maltitol, erythritol, lactitol or isomalt, further optionally the polyol is sorbitol. In an embodiment, the pharmaceutical composition/formulation comprises about 1-10%, 2-5% or 3% polyol by weight, optionally about 3% sorbitol or mannitol or isomalt by weight.

In an embodiment, the pharmaceutical composition/formulation comprises a 5-MeO-DMT salt. In an embodiment, the pharmaceutical composition/formulation comprises 5-MeO-DMT benzoate. In an embodiment, the pharmaceutical composition/formulation comprises 5-MeO-DMT hydrochloride. In an embodiment, the pharmaceutical composition/formulation comprises 5-MeO-DMT hydrobromide. In an embodiment, the 5-MeO-DMT salt is amorphous. In an embodiment, the 5-MeO-DMT salt is crystalline. In an embodiment, the crystalline 5-MeO-DMT salt is selected from: a crystalline form of 5-MeO-DMT benzoate, characterised by one or more peaks in an XRPD diffractogram at 17.5, 17.7 and 21.0°2θ±0.1°2θ as measured using an x-ray wavelength of 1.5406 Å; a crystalline form of 5-MeO-DMT hydrochloride, characterised by one or more peaks in an XRPD diffractogram at 9.2°±0.1°, 12.2°±0.1°, 14.1°±0.1°, 15.0°±0.1°, 18.5°±0.1°, and 19.5°±0.1°2θ as measured by x-ray powder diffraction using an x-ray wavelength of 1.5406 Å; or a crystalline form of 5-MeO-DMT hydrobromide, characterised by one or more peaks in an XRPD diffractogram at 14.6, 16.8, 20.8, 24.3, 24.9 and 27.5° 2θ±0.1°2θ as measured by x-ray powder diffraction using an x-ray wavelength of 1.5406 Å.

In an embodiment, the active nasal delivery device comprises: a dispenser outlet; an air expeller for generating a flow of air while the device is being actuated; and at least one reservoir that contains a single dose of formulation.

In an embodiment, the active nasal delivery device comprises: a dispenser outlet; an air expeller for generating a flow of air while the device is being actuated, said air expeller including a piston that slides in an air chamber between a rest position and a dispensing position, said air chamber including a cylindrical body in which said piston slides in airtight manner; and at least one reservoir that contains a single dose of formulation, said reservoir including an air inlet that is connected to said air expeller, and a pharmaceutical composition/formulation outlet that is connected to said dispenser outlet, said air inlet including a pharmaceutical composition/formulation retainer member for retaining the pharmaceutical composition/formulation in the reservoir until the pharmaceutical composition/formulation is dispensed, and said pharmaceutical composition/formulation outlet being closed by a closure element that is force fitted in the pharmaceutical composition/formulation outlet of the reservoir; said device further including a mechanical opening system that co-operates with said closure element so as to expel said closure element mechanically from a closed position while the device is being actuated, said piston of said air expeller, when in the rest position, co-operating in non-airtight manner with said air chamber, in such a manner that said air chamber is in communication with the atmosphere in the rest position, wherein said piston includes an inner lip configured to cooperate with a cylindrical surface of a cylindrical member extending inside the cylindrical body, said cylindrical surface including fluting that co-operates in non-airtight manner with said inner lip of the piston in the rest position.

In an embodiment, the active nasal delivery device is for use in a method of treating a disease or condition. In an embodiment, the active nasal delivery device is for use in treating a mental health condition, optionally depression and/or alcohol use disorder. Herein disclosed, there is provided a dry powder formulation, produced by spray drying, lyophilisation or hot melt extrusion, wherein the pharmaceutical composition/formulation comprises 5-MeO-DMT, or a pharmaceutically acceptable salt thereof, and one or more pharmaceutically acceptable carriers or excipients. Beneficially, spray drying, lyophilisation or hot melt extrusion provide an intimate mix of the 5-MeO-DMT salt with any of the carriers, excipients or any other additives.

In a first aspect, there is provided a state-stable amorphous dry powder pharmaceutical composition/formulation comprising 5-MeO-DMT HBr (the hydrobromide salt of 5-MeO-DMT) or 5-MeO-DMT HCl (the hydrochloride salt of 5-MeO-DMT) and one or more pharmaceutically acceptable carriers or excipients. Beneficially, the amorphous state does not revert to a crystalline form, and so the nature of the pharmaceutical composition/formulation is well understood. In an embodiment, the pharmaceutical composition/formulation is a spray dried formulation. In an embodiment, no more than 80% of the 5-MeO-DMT is released from the pharmaceutical composition/formulation by 4 minutes in water at 37 °C. In an embodiment, no more than 80% of the 5-MeO-DMT is released from the pharmaceutical composition/formulation by 5, 6, 7, 8, 9 or 10 minutes in water at 37 °C. In an embodiment, no more than 80% of the 5-MeO-DMT is released from the pharmaceutical composition/formulation by 5, 6, 7, 8, 9 or 10 minutes in a simulated nasal fluid. Beneficially, the 5-MeO-DMT is not released immediately, and indeed released relatively slowly. Beneficially, the person being treated receives the active substance over a duration of time. On some occasions, receiving the active substance, a psychoactive substance, over a very short period of time can be quite intense.

In an embodiment, at least 95% of the particles of the pharmaceutical composition/formulation are larger than 10 microns in size. Beneficially, the pharmaceutical composition/formulation does not substantially contain respirable fines (undesirable particles that could enter the lungs). In an embodiment, the 5-MeO-DMT HBr is non-hygroscopic. In an embodiment, the pharmaceutical composition/formulation comprises below about 5% moisture content by weight of the formulation. In an embodiment, the pharmaceutical composition/formulation is a free flowing formulation. Beneficially, non-hygroscopic salts are easy to handle and formulate, these tend to be free-flowing, and are less prone to deformulation.

In an embodiment, greater than 70% (w/w) of the 5-MeO-DMT HBr in the pharmaceutical composition/formulation is in an amorphous form. In an embodiment, the pharmaceutical composition/formulation comprises at least about 10%, 20%, 30%, 40%, 50%, 60%, 70%, 80%, 90%, 95% or 99% by weight 5-MeO-DMT HBr. In an embodiment, greater than 70% (w/w) of the 5-MeO-DMT HCl in the pharmaceutical composition/formulation is in an amorphous form. In an embodiment, the pharmaceutical composition/formulation comprises at least about 10%, 20%, 30%, 40%, 50%, 60%, 70%, 80%, 90%, 95% or 99% by weight 5-MeO-DMT HCl.

In an embodiment, upon administration to a nasal cavity of a subject the pharmaceutical composition/formulation exhibits a residence time. In an embodiment, the length of time a substance is present in nasal cavity, for example along the nasal cilia and mucus layer, in the nasal cavity of at least 10, 15, 20, 25, 30, 35, 40, 45, 50, 55 or 60 minutes. Beneficially, the person being treated receives the active substance over a duration of time. On some occasions, receiving the active substance, a psychoactive substance, over a very short period of time can be quite intense. In an embodiment, the pharmaceutical composition/formulation comprises a cellulose like/based excipient; optionally cellulose ethers, optionally HPMC, further optionally a high viscosity HPMC, still further optionally a high viscosity HPMC. In an embodiment, the pharmaceutical composition/formulation comprises a low viscosity HPMC and a high viscosity HPMC. In an embodiment, the ratio of the low viscosity HPMC to high viscosity HPMC is in the ratio of 1:10 to 10:1, optionally 1:4 to 4:1 and further optionally 1:2 to 2:1. In an embodiment, the ratio of the low viscosity HPMC to high viscosity HPMC is in the ratio of 50:50, 45:55, 40:60, 35:65, 30:70 or 25:75. In an embodiment, the high viscosity HPMC has a viscosity greater or equal to about 20, 30, 40, 50 or 60 megaPascals, optionally where the HPMC is a HPMC containing about 7.0-12.0% hydroxypropyl content, about 28.0-30.0% methoxy content, and a viscosity of about 50 mPas. In an embodiment, the low viscosity HPMC has a viscosity less than about 20, 15, 10, 5, 1 megaPascals, optionally where the HPMC is a HPMC containing about 7.0-12.0% hydroxypropyl content, about 28.0-30.0% methoxy content, and a viscosity of about 4.8-7.2 mPas. In an embodiment, the pharmaceutical composition/formulation comprises a polyol, optionally the polyol is mannitol, xylitol, sorbitol, maltitol, erythritol, lactitol or isomalt, further optionally the polyol is sorbitol. In an embodiment, the pharmaceutical composition/formulation comprises about 1-10%, 2-5% or 3% polyol by weight, optionally about 3% sorbitol by weight.

In an embodiment, the pharmaceutical composition/formulation comprises one or more of: chitosan, chitosan derivatives, β-cyclodextrin, Clostridium perfringens enterotoxin, zonula occludens toxin (ZOT), human neutrophil elastase inhibitor (ER143), sodium taurocholate, sodium deoxycholate sodium, sodium lauryl sulphate, glycodeoxycholate, palmitic acid, palmitoleic acid, stearic acid, oleyl acid, oleyl alcohol, capric acid sodium salt, DHA, EPA, dipalmitoyl phosphatidyl choline, soybean lecithin, lysophosphatidylcholine, dodecyl maltoside, tetradecyl maltoside, EDTA, lactose, cellulose, and citric acid. In an embodiment, the pharmaceutical composition/formulation comprises one or more of: mucoadhesive enhancer, penetrating enhancer, cationic polymers, cyclodextrins, Tight Junction Modulators, enzyme inhibitors, surfactants, chelators, and polysaccharides.

In an embodiment, the pharmaceutical composition/formulation comprises one or more anti-caking agents. In an embodiment, the pharmaceutical composition/formulation comprises one or more of: calcium silicate, sodium aluminosilicate, sodium ferrocyanide, potassium ferrocyanide, calcium carbonate, magnesium carbonate, silicon dioxide, tricalcium phosphate, powdered cellulose, magnesium stearate, sodium bicarbonate, sodium ferrocyanide, potassium ferrocyanide, calcium ferrocyanide, microcrystalline cellulose, calcium phosphate, sodium silicate, silicon dioxide, calcium silicate, magnesium trisilicate, talcum powder, sodium aluminosilicate, potassium aluminium silicate, calcium aluminosilicate, bentonite, aluminium silicate, stearic acid, sodium stearyl fumarate or polydimethylsiloxane.

In an embodiment, there is provided a nasal delivery device comprising the formulation. In an embodiment, the proportion of the active substance (5-MeO-DMT), in the active substance salt is greater than about 65, 70 or 80% (i.e. using a counter ion of smaller relative molecular mass). Beneficially, in a device with a small delivery chamber, a high proportion of active in the salt is desirable. Similarly, the higher the proportion of the salt in the particles is desirable.

In an embodiment, there is provided a method of treating depression and/or alcohol use disorder in a subject in need thereof, the method comprising intranasally administering to the subject the pharmaceutical composition/formulation in an amount sufficient to treat the depression and/or alcohol use disorder. In an embodiment, there is provided a method of making the formulation, the method comprising (i) mixing the components of the pharmaceutical composition/formulation with a liquid to form a mixture and spray drying the mixture to form a solid; and (ii) following following step (i), further drying the solid to form the pharmaceutical composition/formulation and optionally wherein the drying step is performed at between 45 and 15 °C and between 85% to 65% relative humidity (RH); between 35 and 20 °C and between 80% to 70% RH; and further optionally at 25 °C and 75% RH.

In an embodiment, the pharmaceutical composition/formulation comprises about: 40-60% by weight 5-MeO-DMT or pharmaceutically acceptable salt; 30-40% by weight a HPMC containing about 7.0-12.0% hydroxypropyl content, about 28.0-30.0% methoxy content, and a viscosity of about 4.8-7.2 mPas; 7-15% by weight a HPMC containing about 7.0-12.0% hydroxypropyl content, about 28.0-30.0% methoxy content, and a viscosity of about 50 mPas; and 0-5% by weight of sorbitol. In a further embodiment, the pharmaceutical composition/formulation comprises about: 40-60% by weight 5-MeO-DMT HBr or 5-MeO-DMT HCl; 30-40% by weight a HPMC containing about 7.0-12.0% hydroxypropyl content, about 28.0-30.0% methoxy content, and a viscosity of about 4.8-7.2 mPas; 7-15% by weight a HPMC containing about 7.0-12.0% hydroxypropyl content, about 28.0-30.0% methoxy content, and a viscosity of about 50 mPas; and 0-5% by weight of sorbitol.

In an embodiment, the 5-MeO-DMT, or a pharmaceutically acceptable salt thereof, is in an amorphous (non-crystalline) form. In an embodiment, the pharmaceutical composition/formulation is a stable free flowing formulation. In an embodiment, the pharmaceutical composition/formulation is a state-stable free flowing formulation. In an embodiment, the pharmaceutical composition/formulation comprises at least about 10%, 20%, 30%, 40%, 50%, 60%, 70%, 80%, 90%, 95% or 99% by weight 5-MeO-DMT, or a pharmaceutically acceptable salt thereof. In an embodiment, the pharmaceutical composition/formulation exhibits an extended release profile, optionally having a residence time in the nasal cavity of at least 10, 15, 20, 25 or 30 minutes. In an embodiment, the pharmaceutical composition/formulation exhibits an extended release profile, wherein 80% of the 5-MeO-DMT active agent dissolves over a period of time of about 2 to 40, optionally 3 to 30, further optionally 4 to 15 minutes.

In an embodiment, the pharmaceutical composition/formulation comprises a cellulose like/based excipient; optionally cellulose ethers, optionally HPMC, further optionally a high viscosity HPMC, still further optionally a high viscosity HPMC. In an embodiment, the pharmaceutical composition/formulation exhibits an extended release profile. In an embodiment, the pharmaceutical composition/formulation comprises a cellulose like/based excipient; optionally HPMC, further optionally a high viscosity HPMC, still further optionally a high viscosity HPMC, and wherein the pharmaceutical composition/formulation exhibits an extended release profile. In an embodiment, the pharmaceutical composition/formulation exhibits an extended release profile when compared with a pharmaceutical composition/formulation without the cellulose like/based excipient. In an embodiment, the pharmaceutical composition/formulation comprises a low viscosity HPMC and a high viscosity HPMC. In an embodiment, the pharmaceutical composition/formulation exhibits an extended release profile.

In an embodiment, the pharmaceutical composition/formulation comprises a low viscosity HPMC and a high viscosity HPMC, and wherein the pharmaceutical composition/formulation exhibits an extended release profile. In an embodiment, the ratio of the low viscosity HPMC to high viscosity HPMC is in the ratio of 1:10 to 10:1, optionally 1:4 to 4:1 and further optionally 1:2 to 2:1. In an embodiment, the ratio of the low viscosity HPMC to high viscosity HPMC is in the ratio of 50:50, 45:55, 40:60, 35:65, 30:70 or 25:75. In an embodiment, the ratio of the low viscosity HPMC to high viscosity HPMC is in the ratio of 50:50, 45:55, 40:60, 35:65, 30:70 or 25:75 and wherein the pharmaceutical composition/formulation exhibits an extended release profile. In an embodiment, wherein the high viscosity HPMC has a viscosity greater or equal to about 20, 30, 40, 50 or 60 megaPascals, optionally where the HPMC is metolose 60SH50.

In an embodiment, the low viscosity HPMC has a viscosity less than about 20, 15, 10, 5, 1 megaPascals, optionally where the HPMC is pharmacoat 606. In an embodiment, the pharmaceutical composition/formulation comprises a polyol, optionally the polyol is mannitol, xylitol, sorbitol, maltitol, erythritol, lactitol or isomalt, further optionally the polyol is sorbitol. In an embodiment, the pharmaceutical composition/formulation comprises about 1-10%, 2-5% or 3% polyol by weight, optionally about 3% sorbitol by weight.

In an embodiment, the pharmaceutical composition/formulation comprises 5-MeO-DMT hydrochloride, optionally the salt is in an amorphous form, further optionally a state-stable amorphous form. In an embodiment, the pharmaceutical composition/formulation comprises 5-MeO-DMT hydrobromide, optionally the salt is in an amorphous form, further optionally a state-stable amorphous form. In an embodiment, the pharmaceutical composition/formulation comprises 5-MeO-DMT benzoate, optionally the salt is in an amorphous form, further optionally a state-stable amorphous form. In an embodiment, the pharmaceutical composition/formulation comprises 5-MeO-DMT oxalate, optionally the salt is in an amorphous form, further optionally a state-stable amorphous form. In an embodiment, the pharmaceutical composition/formulation comprises 5-MeO-DMT phosphate, optionally the salt is in an amorphous form, further optionally a state-stable amorphous form. In an embodiment, the pharmaceutical composition/formulation comprises 5-MeO-DMT fumarate, optionally the salt is in an amorphous form, further optionally a state-stable amorphous form.

In an embodiment, the pharmaceutical composition/formulation comprises a 5-MeO-DMT salt, optionally the salt is in an amorphous form, further optionally a state-stable amorphous form. In a further aspect of the invention, there is provided a nasal delivery device comprising the pharmaceutical compositions or formulations herein described, and/or in any aspect or embodiment of the invention. In an embodiment, the device is single use. In an embodiment, the device contains a single dose of the formulation. In an embodiment, the pharmaceutical composition/formulation or nasal delivery device is for use as a medicament.

In an embodiment, the pharmaceutical composition/formulation or nasal delivery device is for use in a method of treatment of depression and/or alcohol use disorder. In an embodiment, the pharmaceutical composition/formulation is produced by spray drying and wherein following the spray drying of the formulation, an additional conditioning step is performed to condition the formulation. In an embodiment, the drying step is performed at between 45 and 15 °C and between 85% to 65% relative humidity (RH); optionally between 35 and 20 °C and between 80% to 70% RH; and further optionally at 25 °C and 75% RH.

In an embodiment, there is provided a dry powder formulation, produced by a method of spray drying, wherein the pharmaceutical composition/formulation comprises about: 50% by weight 5-MeO-DMT, or a pharmaceutically acceptable salt thereof; 35% by weight HPMC 606; 12% by weight Metolose 60 SH 50; and 3% by weight of sorbitol.

Liquid intranasal administration is one way of providing systemic drug delivery across the blood brain barrier. However, one of the challenges faced with these liquid pharmaceutical compositions or formulations is the limited residence time in the nasal cavity. The mucociliary clearance mechanism is responsible for this limited residence time, the movement of the nasal cilia leads the upper gel-like mucus layer in the epithelia to move with a velocity of about 6 mm/min towards the nasopharynx and throat. As such, such liquid pharmaceutical compositions or formulations are rapidly removed from the nasal cavity. In addition, some liquid intranasal pharmaceutical compositions or formulations of 5-MeO-DMT, can have stability issues, e.g. discoloration and/or a reduction in desirable pharmacokinetic properties.

Some attempts have been made to develop dry powder pharmaceutical compositions or formulations for intranasal administration which overcome the problems associated with liquid pharmaceutical compositions or formulations. However, this has proved challenging. The first intranasal dry powder pharmaceutical compositions or formulations were approved by the Food and Drug Administration in 2016 and 2019 for Onzetra (containing Sumatriptan) and Baqsimi (containing Glucagon) respectively. Onzentra uses a passive administration device and Baqsimi uses an active device; and so work was needed to find a pharmaceutical composition/formulation and device that in each case would work well in concert.

In addition, some 5-MeO-DMT salts have been found to be very soluble in water (e.g. some crystalline forms of the halide salts of 5-MeO-DMT have a solubility of >400 mg/ml in water) and have very rapid dissolution profiles. While these properties can be desirable in solid oral or intravenous dosage pharmaceutical compositions or formulations, they are not necessarily beneficial for intranasal pharmaceutical compositions or formulations of 5-MeO-DMT.

This is because 5-MeO-DMT can provide a very intense Mystical Experience in a subject. So, for a highly soluble intranasal pharmaceutical composition/formulation that rapidly crosses the blood brain barrier (e.g. 80% of the dose of the active agent in under about 4 minutes) the Mystical Experience generated may happen very quickly, and can be quite intense, cause irritation, and this may be unsettling for some users.

The applicant has beneficially found that dry powder pharmaceutical compositions or formulations of 5-MeO-DMT (and the salts described herein), in particular amorphous dry powder pharmaceutical compositions or formulations of the same, address and/or ameliorate the problems encountered in the prior art as discussed further herein below.

The applicant has beneficially found that the following factors (not necessary listed in the order of importance) provide beneficial properties when making dry powder pharmaceutical compositions or formulations of 5-MeO-DMT: amorphous (non-crystalline) form/state; moderate/lower solubility forms; excipients/agents that slow/retard the dissolution of the active agent across the nasal blood brain barrier (e.g. in particular cellulose like/based excipients like HMPC and (methyl) cellulose). As such, the applicant has sought to increase the residency time of the active agent (5-MeO-DMT) in the nasal cavity. Other modifications have not proved effective and indeed in some cases have lessened the residency time in the nasal cavity. Also, when matching the pharmaceutical composition/formulation to a dry powdered delivery device, the proportion of the active agent in the pharmaceutical composition/formulation should be relatively high (so a smaller formula weight counter ion can be beneficial, as is a low proportion of any excipient/additives), e.g. to maximise the proportion of the active agent in the delivery vehicle, which may need to fit within a relatively small delivery chamber in the delivery vehicle (e.g. holding a volume of less than about 0.05 ml). Also, beneficially, the pharmaceutical composition/formulation should be state-stable e.g. it should not (re) crystalize, in particular upon storage. More beneficially, an amorphous (non-crystalline) form/state should be state-stable at or above room temperature e.g. it should not (re) crystalize when stored at about above 0, 5, 10, 15, 20, 25, 30 or 35 °C. Also, the cellulose like/based excipients may have a viscosity to suit need, e.g. high, moderate or low viscosity or contain a mixture of these with different viscosities (e.g. a high and low viscosity). Without being bound by theory, it is possible that the 5-MeO-DMT is preferentially soluble in the cellulose like/based excipients, and this delays/retards the API (i.e. 5-MeO-DMT) travelling across the nasal blood brain barrier. Advantageously, the pharmaceutical composition/formulation should be free from respirable API fines (e.g. which may enter the lungs) and/or significant amounts of any aggregation. Respirable fines are particles which are 10 microns or less. In an embodiment, the pharmaceutical composition/formulation comprises particles which are larger than 10 microns. In an embodiment, the pharmaceutical composition/formulation is substantially free of respirable fines. In an embodiment, 75, 80, 90, 95, 98, 99, 99.5, 99.8 or 99.9% of the particles of the pharmaceutical composition/formulation are larger than 10 micrometres.

In an embodiment the pharmaceutical composition/formulation comprises particles having a median diameter of less than 2000 μm, 1000 μm, 500 μm, 250 μm, 100 μm, 50 μm, or 1 μm. In an embodiment, the particles have a median diameter of greater than 500 μm, 250 μm, 100 μm, 50 μm, 40 μm, 30 μm, 25 μm, 20 μm, 15 μm, 10 μm, 5 μm, 1 μm or 0.5 μm. In an embodiment pharmaceutical composition/formulation has a particle size distribution of d10=20-60 μm, and/or d50=80-120 μm, and/or d90=130-300 μm.

It is considered that a pharmaceutical composition/formulation could be prepared that contained a slow release element/portion and a standard release element/portion (e.g. a mixture of amorphous and partially crystalline 5-MeO-DMT (the API)), such that there is an initial release of the API (e.g. a lower amount), followed by a slower release of the API (e.g. a larger amount). In an embodiment, the pharmaceutical composition/formulation comprises crystalline API dry blended with SDD comprising API/HPMC. Beneficially, this may provide for higher drug loadings within a single device.

Herein disclosed, the pharmaceutical composition/formulation is free from the one or more pharmaceutically acceptable carriers or excipients. 5-MeO-DMT is very likely to be treated as a controlled drug by Health Authorities. The Authorities would require the setting up of specific methodology for the prescribing and use of this drug in patients. When taken as prescribed by a licensed physician, controlled substances can effectively treat many conditions. However, over the past decade, in many countries, it can be seen that a drastic increase in controlled substance diversion, misuse, and abuse has occurred.

Most drugs of abuse directly or indirectly target the brain's reward system affecting the body's ability to regulate movement, emotion, motivation, and feelings of pleasure. When the system is overstimulated with certain controlled substances, it produces euphoric effects, which strongly reinforce the behaviour of substance use, thereby teaching the user to repeat the action. Although the leading route of administration is oral, some individuals alter the route of administration to intensify the effect or as they build a tolerance to the substance.

Alternative routes of administration, including intranasal (e.g. crushing and snorting) and intravenous (e.g. dissolving and injecting) methods, smoking inhalation, often provide faster drug delivery and onset, and intensified effects. Individuals who use altered routes of administration are at an increased risk for overdose and the development or exacerbation of substance use disorders. Abuse-deterrent pharmaceutical compositions or formulations prevent inexperienced substance users from successfully ingesting substances via altered routes of administration, and thereby prevent associated overdoses and escalation of substance use, resulting in significant personal and public health benefits.

Abuse-deterrent pharmaceutical compositions or formulations limit one or more forms of such abuse by reducing the attractiveness or drug-liking qualities of a controlled substance by: (1) Hindering the extraction of active ingredients; (2) Decreasing their bioavailability through product manipulation, thereby reducing the feeling of euphoria; (3) Preventing administration through alternative routes; (4) Making abuse of the manipulated product less attractive or rewarding.

In an embodiment the 5-MeO-DMT pharmaceutical composition/formulation is formulated as an abuse-deterrent pharmaceutical composition/formulation to include one or more of the following types of abuse deterrent technology: (1) Physical barriers: to prevent chewing, crushing, cutting, grating or grinding; (2) Chemical barriers: to resist extraction of the opioid using common solvents, such as water, alcohol, or other organic solvents; (3) Agonist/antagonist combinations: to interfere with, reduce, or defeat the euphoria associated with abuse upon manipulation of the product; (4) Aversion: to combine substances in order to produce an unpleasant effect if the dosage form is manipulated prior to ingestion or a higher dosage than directed is used; (5) Delivery systems: certain drug release designs or methods of drug delivery that offer resistance to abuse, such as depot injectable pharmaceutical compositions or formulations or implants; 6) Prodrugs: to lack activity until transformed in the gastrointestinal tract, thereby making intravenous injection or intranasal abuse less attractive; (7) Combination: products that combine two or more of the above-mentioned methods.

Particular 5-MeO-DMT pharmaceutical compositions or formulations as described herein in the form of, for example, spray dried dispersions of 5-MeO-DMT in combination with one or more pharmaceutically acceptable excipients benefit not only from the herein described advantageous properties of said dispersions, but also from the fact that said dispersions provide a deterrent to abuse. The intimately mixed nature of the dispersions provides a barrier to the easy extraction of the active pharmaceutical ingredient.

In an embodiment, there is provided a 5-MeO-DMT pharmaceutical composition/formulation as described herein wherein said pharmaceutical composition/formulation comprises a polyol and wherein said pharmaceutical composition/formulation shows a strong reduction of mucosal irritation upon the intranasal, buccal, sublabial or sublingual administration thereof, compared with a pharmaceutical composition/formulation without a polyol.

In an embodiment, there is provided a method of reducing mucosal irritation in a subject upon intranasal, buccal, sublabial or sublingual administration of 5-MeO-DMT to the subject, wherein said method comprises providing the 5-MeO-DMT in a pharmaceutical composition/formulation as described herein, wherein said pharmaceutical composition/formulation comprises a polyol. In an embodiment, there is provided a 5-MeO-DMT pharmaceutical composition/formulation as described herein wherein said pharmaceutical composition/formulation comprises 5-MeO-DMT as the saccharinate salt and wherein said pharmaceutical composition/formulation shows a strong reduction of mucosal irritation upon the intranasal, buccal, sublabial or sublingual administration thereof, compared with a pharmaceutical composition/formulation of 5-MeO-DMT freebase or an alternative salt form.

In an embodiment, there is provided a method of reducing mucosal irritation in a subject upon intranasal, buccal, sublabial or sublingual administration of 5-MeO-DMT to the subject, wherein said method comprises providing the 5-MeO-DMT in a pharmaceutical composition/formulation as described herein, wherein said pharmaceutical composition/formulation 5-MeO-DMT as the saccharinate salt. In an embodiment, there is provided a pharmaceutical composition/formulation comprising 5-MeO-DMT, or a pharmaceutically acceptable salt thereof, wherein said pharmaceutical composition/formulation is as described herein and/or is produced as described herein and has a residency time in the nasal cavity of greater than or equal to 5, 6, 7, 8, 9, 10, 11, 12, 13, 14, 15, 16, 17, 18, 19, 20, 25, 30, 35, 40, 45, 50, 55 or 60 minutes.

In an embodiment, the pharmaceutical composition/formulation comprises a salt of 5-MeO-DMT. In an embodiment, the pharmaceutical composition/formulation comprises a crystalline salt of 5-MeO-DMT. In an embodiment, there is provided a crystalline form of 5-MeO-DMT hydrobromide. In an embodiment, there is provided a crystalline form of 5-MeO-DMT hydrobromide, characterised by one or more peaks in an XRPD diffractogram at 14.6, 16.8, 20.8, 24.3, 24.9 and 27.5° 2θ±0.1°2θ as measured by x-ray powder diffraction using an x-ray wavelength of 1.5406 Å. In an embodiment, there is provided a crystalline form of 5-MeO-DMT phosphate, characterised by one or more peaks in an XRPD diffractogram at 12.9, 20.4 and 23.1° 2θ±0.1°2θ as measured by x-ray powder diffraction using an x-ray wavelength of 1.5406 Å. In an embodiment, there is provided a crystalline form of 5-MeO-DMT fumarate, characterised by one or more peaks in an XRPD diffractogram at 13.0, 16.3 and 22.1° 2θ±0.1°2θ as measured by x-ray powder diffraction using an x-ray wavelength of 1.5406 Å. In an embodiment, there is provided a crystalline form of 5-MeO-DMT oxalate, characterised by one or more peaks in an XRPD diffractogram at 13.0, 19.9 and 26.0° 2θ±0.1°2θ as measured by x-ray powder diffraction using an x-ray wavelength of 1.5406 Å. In an embodiment, there is provided a crystalline form of 5-MeO-DMT tartrate, characterised by one or more peaks in an XRPD diffractogram at 18.3, 18.6, and 20.7° 2θ±0.1°2θ as measured by x-ray powder diffraction using an x-ray wavelength of 1.5406 Å. In an embodiment, there is provided a crystalline form of 5-MeO-DMT benzenesulfonate, characterised by one or more peaks in an XRPD diffractogram at 9.5, 21.2, and 23.6° 2θ±0.1°2θ as measured by x-ray powder diffraction using an x-ray wavelength of 1.5406 Å. In an embodiment, there is provided a crystalline form of 5-MeO-DMT tosylate, characterised by one or more peaks in an XRPD diffractogram at 19.3, 23.6 and 24.1°2θ±0.1°2θ as measured by x-ray powder diffraction using an x-ray wavelength of 1.5406 Å. In an embodiment, there is provided a crystalline form of 5-MeO-DMT glycolate, characterised by one or more peaks in an XRPD diffractogram at 20.2, 21.1 and 23.4° 2θ±0.1°2θ as measured by x-ray powder diffraction using an x-ray wavelength of 1.5406 Å. In an embodiment, there is provided a crystalline form of 5-MeO-DMT ketoglutarate, characterised by one or more peaks in an XRPD diffractogram at 14.4, 18.2 and 20.9°2θ±0.1°2θ as measured by x-ray powder diffraction using an x-ray wavelength of 1.5406 Å. In an embodiment, there is provided a crystalline form of 5-MeO-DMT malate, characterised by one or more peaks in an XRPD diffractogram at 18.3, 18.7 and 18.9°2θ±0.1°2θ as measured by x-ray powder diffraction using an x-ray wavelength of 1.5406 Å. In an embodiment, there is provided a crystalline form of 5-MeO-DMT saccharinate, characterised by one or more peaks in an XRPD diffractogram at 8.7, 15.2 and 20.9° 2θ±0.1°2θ as measured by x-ray powder diffraction using an x-ray wavelength of 1.5406 Å. In an embodiment, there is provided a crystalline form of 5-MeO-DMT hydrochloride, characterised by one or more peaks in an XRPD diffractogram at 9.2°±0.1°, 12.2°±0.1°, 14.1°±0.1°, 15.0°±0.1°, 18.5°±0.1°, and 19.5°±0.1° 20 as measured by x-ray powder diffraction using an x-ray wavelength of 1.5406 Å. In an embodiment, there is provided a crystalline form of 5-MeO-DMT benzoate, characterised by one or more peaks in an XRPD diffractogram at 17.5, 17.7 and 21.0° 2θ±0.1°2θ as measured using an x-ray wavelength of 1.5406 Å.

### DESCRIPTION OF THE INVENTION

In an embodiment, there is provided a dry powder pharmaceutical composition/formulation comprising 5-MeO-DMT, or a pharmaceutically acceptable salt thereof, and polyvinylpyrrolidone (PVP).

Herein disclosed, there is provided a method of production of a pharmaceutical composition/formulation of 5-MeO-DMT comprising the steps of: Atomisation of a liquid mixture comprising 5-MeO-DMT to produce droplets; Contact between a hot gas and the droplets to dry the droplets; Optionally, the separation of the dried product from the drying medium; and Conditioning of the dried product.

In an embodiment, the conditioning step comprises exposing the dried product to between 15 to 45 °C and between 65% to 85% relative humidity (RH); optionally between 2° and 35 °C and between 70 to 80% RH; and further optionally 25 °C and 75% RH.

In an embodiment, the conditioning step takes place for between 1 day and several weeks. In an embodiment, the conditioning step takes place for between 1 day and 1 week. In an embodiment, the conditioning step takes place for between 1 day and 3 days. In an embodiment, the conditioning step takes place for less than 1 day.

In an embodiment, there is provided a pharmaceutical composition/formulation as described previously or subsequently. In an embodiment, the pharmaceutical composition/formulation is produced by spray drying, lyophilisation and/or hot melt extrusion.

Unless otherwise stated herein all X-ray powder diffractograms (XRPD) were generated using an X-ray wavelength of 1.5406 Å; all modulated differential scanning calorimetry (DSC) thermograms were generated using a 2 °C/min heating rate.

The applicant has beneficially found that in order to slow the dissolution rate, and therefore increase the residency time in the nasal cavity, the dry powder pharmaceutical composition/formulation should optionally include one or more suitable excipients.

Hydroxypropyl methylcellulose (HPMC) or hypromellose refers to soluble methylcellulose ethers and is approved as an inactive ingredient. Without being bound by theory, HPMC is believed to act as a viscosity enhancer, and delays/slows mucociliary clearance. HPMC polymers for fabricating hydrophilic matrix systems are available in various viscosity grades ranging from 4000-100,000 mPas. The polymer chain length, size and degree of branching determine the viscosity of the polymer in solution.

Different grades of HPMC (with lower viscosities than that above) are also available according to their particle size distribution, viscosity, molecular weights, and substitution of methoxy and hydroxypropyl groups.

For the purposes of this application a high viscosity HPMC would generally be considered to be one with a viscosity grade of 20 mPas or above. For the purposes of this application a low viscosity HPMC would generally be considered to be one with a viscosity grade of less than 20 mPas.

HPMC 2910 has an average content of methoxy groups of 29% and hydroxypropoxy groups of 10% (hence the nomenclature of 2910). Pharmacoat is a brand of low viscosity HPMC 2910, with Pharmacoat 606 (as commercially available in the UK as of 1 Jun. 2023) has a viscosity of 6 mPas. Metolose is a brand of high viscosity HPMC 2910 and methyl cellulose. Metolose 60SH50 606 (as commercially available in the UK as of 1 Jun. 2023) has a viscosity of 50 mPas

### Dry Blending

Dry blending (giving a solid dispersion matrix) of cellulose based excipients, such as HPMC 2910, with the API at high concentrations up to approximately 95% wt:wt (excipient to 5-MeO-DMT) in the blend, beneficially slowed the dissolution release rate of the API relative to an unblended formulation.

Care is needed when making dry blends to ensure blend uniformity and monitoring of any aggregation/agglomeration may be needed. Also, the nature of the blend needs to be assessed to ensure the physical properties work well with the delivery device and with scale up, e.g. if the resultant blend is prone to static charge build up, then the blend can be difficult to load into the delivery device without losses.

The applicant also found that an intimate mix pharmaceutical composition/formulation of the API with excipients gave beneficial properties. Intimate mix pharmaceutical composition/formulation (e.g. spray drying, lyophilisation and/or hot melt extrusion with one or more suitable pharmaceutical excipients or carriers) are considered further below. These techniques are best known for generating amorphous solid dispersions with improved bioavailability and increased solubility. In the present case, with the intense Mystical Experiences associated with the API, this would not on the face of it appear beneficial. Typically such dispersions comprise API loading of around 20% wt:wt (e.g. 5-MeO-DMT to excipients) or below.

### Lyophilisation Pharmaceutical Compositions or Formulations

Amorphous 5-MeO-DMT salt pharmaceutical compositions or formulations have been produced via lyophilisation processes. It is noted that in some cases these have low glass transition temperatures. Conversion to a crystalline form may therefore occur, perhaps rapidly. Solubility studies utilising a lyophilised amorphous form of 5-MeO-DMT benzoate showed an almost instantaneous dissolution rate, which would have applications where this was a desirable property.

### Hot-Melt Pharmaceutical Compositions or Formulations

Hot melt extrusion may be utilised to produce 5-MeO-DMT pharmaceutical compositions or formulations according to the current invention and/or any embodiments thereof. Hot melt extrusion is the processing of polymeric materials above their glass transition temperature (Tg) in order to effect molecular level mixing of thermoplastic binders and/or polymers and active compounds.

### Spray Dried Dispersions

Spray drying typically involves injecting a liquid pharmaceutical composition/formulation of material into a chamber for contact with a drying fluid which is concurrently flowing through the chamber. The injected wet material in the form of droplets contacts the stream of drying fluid so that the liquid passes from the droplets to the drying fluid stream, producing a spray dried product that is discharged from the drying chamber, and drying fluid effluent that likewise is discharged from the drying chamber.

Advantageously, and unexpectedly, the applicant discovered that spray dried dispersions of 5-MeO-DMT gave products with lower/reduced dissolution rates. This was unexpected because spray dried dispersions are generally developed to improve the solubility of low solubility products. The expectation was that the small particles produced by spray drying would have a larger surface area, which allows them to dissolve more easily in the body.

That said, there is a limit to the amount of product that can be administered to the nasal cavity through a medical device, typically below 50 mg (approximately 0.05 ml in volume). In the case of 5-MeO-DMT dry powder pharmaceutical compositions or formulations, it is envisaged that single doses of up to 20 mg or above API may be needed, which may require an API loading of for example 50% wt:wt, levels which are not typically seen in spray dried solid dispersions.

Below, more specific sprayed dried pharmaceutical compositions or formulations are considered.

### 5-MeO-DMT Benzoate|Spray Dried Powder Pharmaceutical Composition/Formulation

Spray dried powder pharmaceutical compositions or formulations containing excipient(s) and the benzoate salt of 5-MeO-DMT were produced, which contained dried particles of suitable size for intranasal administration and surprisingly, a reduced dissolution rate as compared to the amorphous form of 5-MeO-DMT salts. Respirable particles, particles which can penetrate beyond the terminal bronchioles into the gas-exchange region of the lungs are undesirable as they can, for example, trigger bronchoconstriction in asthmatics. In addition, uncontrolled recrystallisation over time within the spray dried dispersion particles may also lead to aggregation of the particles.

With this benzoate salt, the applicant has found that storage at 2-8° C., and protection from moisture, prevents recrystallisation and aggregation. In this case, it was found that a post spray drying step (conditioning the spray dried dispersion particles) at 25 °C/75% RH produced a stable crystalline spray dried dispersion particle with no respirable API fines or significant aggregation.

It was also discovered that producing a spray dried dispersion with reduced amount of the benzoate salt loading (e.g. API loading to approximately 20% wt:wt) produced an amorphous dispersion with no sign of the crystalline benzoate salt. In an embodiment, there is provided a dry powder pharmaceutical composition/formulation of this 5-MeO-DMT salt and one or more pharmaceutically acceptable carriers or excipients. In an embodiment, the pharmaceutical composition/formulation is an amorphous dry powder formulation. In an embodiment, the pharmaceutical composition/formulation has been produced by spray drying. In an embodiment, the pharmaceutical composition/formulation has been produced by lyophilisation. In an embodiment, the pharmaceutical composition/formulation has been produced by hot melt extrusion.

### 5-MeO-DMT Oxalate|Spray Dried Powder Formulation

A spray dried powder pharmaceutical composition/formulation of 5-MeO-DMT oxalate (50% API loading) and excipient(s) was produced and yielded partially crystalline particles (see Example 2). Without being bound by theory, it is believed that lowering the API content (as was the case with the Benzoate salt) could produce an amorphous dispersion with no sign of the crystalline salt.

In an embodiment, there is provided a dry powder pharmaceutical composition/formulation of this 5-MeO-DMT salt and one or more pharmaceutically acceptable carriers or excipients. In an embodiment, the pharmaceutical composition/formulation is an amorphous dry powder formulation. In an embodiment, the pharmaceutical composition/formulation has been produced by spray drying. In an embodiment, the pharmaceutical composition/formulation has been produced by lyophilisation. In an embodiment, the pharmaceutical composition/formulation has been produced by hot melt extrusion.

### 5-MeO-DMT Hydrobromide| Spray Dried Powder Formulation

A spray dried powder pharmaceutical composition/formulation of 5-MeO-DMT hydrobromide (50% API loading) and excipient(s) was produced. Surprisingly, this pharmaceutical composition/formulation was found to be a stable amorphous dispersion without the need for any additional drying step post spray drying. It was also state-stable when stored at above the temperature range 2-8 °C.

In an embodiment, there is provided a dry powder pharmaceutical composition/formulation of this 5-MeO-DMT salt and one or more pharmaceutically acceptable carriers or excipients. In an embodiment, the pharmaceutical composition/formulation is an amorphous dry powder formulation. In an embodiment, the pharmaceutical composition/formulation has been produced by spray drying. In an embodiment, the pharmaceutical composition/formulation has been produced by lyophilisation. In an embodiment, the pharmaceutical composition/formulation has been produced by hot melt extrusion. In an embodiment, the pharmaceutical composition/formulation is non-hygroscopic.

### Extended Release Pharmaceutical Compositions or Formulations of the Salts of 5-MeO-DMT

Recent clinical trials undertaken by the applicant have identified the desirability of an extended release pharmaceutical composition/formulation of 5-MeO-DMT (or salts thereof) suitable for intranasal administration. The applicant has surprisingly discovered that the use of higher viscosity excipients, within a spray dried dispersion as described previously or subsequently, can suitably extend the release window of the API 5-MeO-DMT.

More viscous HPMCs, such as HPMC metolose 60SH50, are not typically suitable for spray drying due to the high viscosity solutions produced as feed stock. Unexpectedly, it has been found that spray drying 5-MeO-DMT with a mixture of a high viscosity HPMC and a lower viscosity HPMC (such as pharmacoat 606) produces spray dried dispersion droplets in which the dissolution of the API from the pharmaceutical composition/formulation is slowed.

Various ratios of high viscosity HPMC to low viscosity HPMC have been investigated and it was discovered that the dissolution rate of the API did not significantly differ between a ratio of 50:50 high:low HPMC and a ratio of 25:75 high:low HPMC. The pharmaceutical compositions or formulations comprising a lower amount overall of high viscosity HPMC are more amenable to the spray drying process (e.g. lower viscosity feed stock).

It was noted that the products obtained with a single HPMC had yields that were higher than products obtained with mixed HPMC products in the spray drying process described above. Further investigation found that, beneficially, that the addition of a polyol to the pharmaceutical composition/formulation with the mixed HPMCs, improved the resultant yields (e.g. by as much as about 18% compared with a pharmaceutical composition/formulation lacking the polyol). Beneficially and unexpectedly, the addition of the polyol has no appreciable effect on the dissolution rate of the API in the formulation.

In an embodiment an additive such as a polyol or surfactant etc. is added to the pharmaceutical composition/formulation prior to spray drying. In an embodiment, the pharmaceutical composition/formulation comprises two (or more) different HPMCs. In an embodiment, the pharmaceutical composition/formulation comprises two (or more) different HPMCs where these have different viscosities. In an embodiment, the pharmaceutical composition/formulation comprises two (or more) different HPMCs. In an embodiment, the pharmaceutical composition/formulation comprises two (or more) different HPMCs where these have different viscosities, and wherein the net viscosity of the mixture is spray dryable. In an embodiment, the pharmaceutical composition/formulation comprises two (or more) different HPMCs where these have different viscosities, wherein the net viscosity of the mixture is spray dryable, and wherein at least one of the HPMCs alone would not be readily suitable for spray drying.

In an embodiment, there is provided an extended release dry powder pharmaceutical composition/formulation of 5-MeO-DMT comprising a mixture of a high viscosity HPMC and a low viscosity HPMC. In an embodiment, the extended release dry powder pharmaceutical composition/formulation comprises a ratio of 1:1 of the high and low HPMC. In an embodiment, the ratio is 1:2 of high to low HPMC. In an embodiment, the ratio is 1:3 of high to low HPMC.

In an embodiment, the pharmaceutical composition/formulation further comprises a polyol (e.g. an organic compound, e.g. 4 to 12 carbon atoms, and containing multiple hydroxyl groups (—OH)). Optionally containing 4 to 6 carbon atoms. Some of these are polyether, polyester, polycarbonate and also acrylic polyols. Polyether polyols may be further subdivided and classified as polyethylene oxide or polyethylene glycol (PEG), polypropylene glycol (PPG) and Polytetrahydrofuran or PTMEG. These have 2, 3 and 4 carbons respectively per oxygen atom in the repeat unit. Polycaprolactone polyols are also available. Polyols may be biobased and hence renewable.

In an embodiment, the polyol is selected from mannitol, xylitol, sorbitol, maltitol, erythritol, lactitol or isomalt. In an embodiment, the polyol is sorbitol. In an embodiment, the pharmaceutical composition/formulation comprises 3% sorbitol. In an embodiment, the extended release dry powder pharmaceutical composition/formulation is produced by spray drying. In an embodiment, the production method comprises addition of a polyol e.g. sorbitol or mannitol or combinations of both (e.g. isomalt).

### Low Crystalline Content Spray Dried Dispersions

Beneficially, the applicant has discovered that reducing the relative 5-MeO-DMT salt loading of the spray dried dispersion pharmaceutical composition/formulation leads to a reduction in salt crystalline content (and hence an overall increase in the amorphous content present in the dispersion). The level of crystalline content was determined using a higher than normal heating rate DSC method and the crystalline content was comparable to that of the low viscosity HPMC pharmaceutical compositions or formulations. The increased amorphous API content in these pharmaceutical compositions or formulations surprisingly led to a reduction in the dissolution rate of the formulation. This is contrary to the expected result, wherein amorphous solid dispersions are manufactured with the aim of enhancing the solubility and dissolution of the formulation.

In an embodiment, the dry powder pharmaceutical composition/formulation has a moisture content of below about 1%, about 2%, about 3%, about 4%, about 5%, about 6%, about 7%, about 8%, about 9%, about 10% by weight of the formulation.

### Non-Hygroscopic 5-MeO-DMT Hydrobromide

In an embodiment, the pharmaceutical composition/formulation is non-hygroscopic. It has been surprisingly found that the hydrobromide salt of 5-MeO-DMT is non-hygroscopic. Hygroscopicity is the phenomenon of attracting and holding water molecules via either adsorption or absorption from the surrounding environment. Pharmaceuticals that pick up less than 0.2% moisture at 80% RH are considered non hygroscopic. Pharmaceuticals that pick up between 0.2% and 2.0% moisture at 80% RH are considered slightly hygroscopic. Pharmaceuticals that pick up between 2.0% and 15.0% moisture at 80% RH are considered moderately hygroscopic. Pharmaceuticals that pick up more than 15.0% moisture at 80% RH are considered very hygroscopic. Hygroscopic substances are difficult to handle and costly and burdensome measures must be taken in order to ensure they are not exposed to moisture during process and formulation. Exposed to moisture, hygroscopic substances can take on water and convert to a hydrous form. This presents several disadvantages. First, the hydrous forms may have the disadvantage of being less bioavailable and less dissoluble than the anhydrous forms. Second, the variation in the amount of hydrous versus anhydrous substance from batch to batch could fail to meet specifications set by drug regulatory agencies. Third, processes like milling may cause the drug substance to adhere to manufacturing equipment which may further result in processing delay, increased operator involvement, increased cost, increased maintenance and lower production yield. Fourth, in addition to problems caused by introduction of moisture during the processing of these hygroscopic substances, the potential for absorbance of moisture during storage and handling would adversely affect the dissolubility of the drug substance. Thus shelf-life of the product could be significantly decreased and/or packaging costs could be significantly increased. Beneficially, the non-hygroscopic properties of the 5-MeO-DMT hydrobromide additionally make it a good salt form for a dry powder formulation.

### Nasal Delivery Devices

In an embodiment, there is provided a nasal powder dispenser device having a reservoir containing at least one dose of powder comprising 5-MeO-DMT, or a pharmaceutically acceptable salt thereof (inclusive of any of the aspects and/or embodiments of the invention and/or pharmaceutical compositions or formulations as described herein). Further, the embodiment may comprise one or more of: a nasal dispenser head for inserting into a user's nostril, the nasal dispenser head including a dispenser orifice; and an air expeller that, during actuation of the nasal powder dispenser device, generates a flow of compressed air so as to dispense a dose of powder into the nostril through the dispenser orifice. In an embodiment, the air expeller has an air chamber and a piston that slides in airtight manner in the air chamber so as to compress the air contained in the air chamber. In an embodiment, in the nasal powder dispenser device, a pressure of the flow of compressed air generated by the air expeller is higher than 0.7 bar; and a volume of the air chamber is greater than about 1700 mm³ (corresponding to a volume of about 12×12×12 mm).

In an embodiment, there is provided a nasal powder delivery device having a container comprising a dose of powder comprising at least particles of 5-MeO-DMT or a pharmaceutically acceptable salt thereof, a nasal delivery head, and an air discharge system generating a flow of compressed air for delivering a dose of powder into the nostril. In an embodiment, the air chamber is arranged in a skirt, and a piston sealingly slides in the air chamber to compress the air. In an embodiment, the piston is connected to an actuating member, in which, before actuation, at least one breakable bridge is provided between the skirt and the actuating member, wherein each breakable bridge is formed on the skirt and cooperates with a radial projection formed on the actuating member. In an embodiment, each radial projection has an axial extension greater than that of the respective breakable bridge and forms an inclined axial ramp on either side.

In an embodiment the device is an Aptar device (UDS-Unidose Solid) as commercially available in the UK as of 1 Jun. 2023. Dry powder devices described in WO21005308; WO22123128; WO22171969; and WO22208014 are incorporated herein by reference. In an embodiment, the counter ion (anion) of the 5-MeO-DMT salt is a benzoate, hydrobromide, hydrochloride, phosphate, fumarate, oxalate, tartrate, benzenesulfonate, tosylate, glycolate, ketoglutarate, malate, saccharinate or succinate salt.

In an embodiment, the pharmaceutical composition/formulation may contain two salts of 5-MeO-DMT, wherein the second salt of 5-MeO-DMT salt is a benzoate, hydrobromide, hydrochloride, phosphate, fumarate, oxalate, tartrate, benzenesulfonate, tosylate, glycolate, ketoglutarate, malate, saccharinate or succinate salt. The salt type can be selected by the medical practitioner or formulator to suit need and the particular circumstance of the patient being treated or the physical requirements of the pharmaceutical composition/formulation needed. In an embodiment one, or both salts, comprise or consist of an amorphous (non-crystalline) state. In an embodiment one, or both salts, comprise or consist of a state-stable amorphous (non-crystalline) state.

In an embodiment (one or both of) the 5-MeO-DMT salt is 5-MeO-DMT benzoate. Advantageously, the benzoate salt has shown good irritation tolerability, in particular when compared to the better known chloride salt, and has a good stability profile. In an embodiment, the 5-MeO-DMT benzoate is not crystalline. In an embodiment, the crystalline 5-MeO-DMT benzoate is characterised by one or more peaks in an X-ray powder diffraction (XRPD) diffractogram at a 20 value of 17.5°±0.1°, 17.7°±0.1° and 21.0°±0.1° using an X-ray wavelength of 1.5406 Å. In an embodiment (one or both of) the 5-MeO-DMT salt is 5-MeO-DMT hydrobromide, and wherein the pharmaceutical composition/formulation comprises substantially the same dosage amount of the active 5-MeO-DMT cation. Advantageously, the hydrobromide salt is substantially non-hygroscopic.

It should be appreciated that different salts of 5-MeO-DMT will have different formula weights. For example the hydrochloride, hydrobromide and benzoate have respectively formula weights of about 254.8 g/mol, 299.2 g/mol, 340.4 g/mol and the free base of 5-MeO-DMT 218.3 g/mol. So, this is the amount of substance that is required to give 1 mol of the active agent. So, for example for the salt, the dosage amount may be the equivalent amount of the free base delivered when the salt is taken. So 100 mg dosage amount of 5-MeO-DMT corresponds to 117 mg of the hydrochloride salt (i.e. both providing the same molar amount of the active substance). The greater mass of the salt needed is due to the larger formula weight of the hydrogen chloride salt (i.e. 218.3 g/mol for the free base as compared to 254.8 g/mol for the salt). Similarly, for a deuterated or triturated version of 5-MeO-DMT (also considered within the scope of the invention), a slight increase in mass can be expected due to the increased formula weight of these isotopic compounds. Unless stated otherwise, the mass (mg) of 5-MeO-DMT refers to the mass of benzoate salt (and so the equivalent molar amount of the 5-MeO-DMT active agent). Accordingly, with reference to the other salts mentioned herein, the appropriate mass of the other salt can be scaled accordingly using ratios of the formula weights. These masses of salts are normally rounded up or down to suit need. This rounding may be to the nearest whole, half, quarter or tenth of a milligram (mg). For example, splitting of a combined dose will typically be done to whole numbers so 3.5 and 6.5 mg (combined total of 10 mg) may be formulated to 3 and 7 mg respectively.

In an embodiment, the pharmaceutical composition/formulation comprises the hydrochloride, phosphate, fumarate, oxalate, tartrate, benzenesulfonate, tosylate, glycolate, ketoglutarate, malate, saccharinate or succinate salt of 5-MeO-DMT. In an embodiment, the pharmaceutical composition/formulation does not comprises a crystalline form of the hydrochloride, phosphate, fumarate, oxalate, tartrate, benzenesulfonate, tosylate, glycolate, ketoglutarate, malate, saccharinate or succinate salt of 5-MeO-DMT.

In an embodiment, the 5-MeO-DMT is administered as the free base. In an embodiment, the 5-MeO-DMT is administered as a salt. In an embodiment, the 5-MeO-DMT is not administered as a crystalline salt. In an embodiment, the 5-MeO-DMT is not administered as a polymorphic salt form. In an embodiment, the 5-MeO-DMT is not administered as a polymorph of a 5-MeO-DMT salt. In an embodiment, the 5-MeO-DMT is administered as the benzoate, fumarate, citrate, acetate, succinate, halide, fluoride, chloride, bromide, iodide, oxalate, or triflate salt. In an embodiment, the 5-MeO-DMT is administered as the benzoate salt. In an embodiment, the 5-MeO-DMT is administered as the hydrochloride salt. In an embodiment, the 5-MeO-DMT is administered as the hydrobromide salt. In an embodiment, the 5-MeO-DMT salt is administered in an amorphous form. In an embodiment, the 5-MeO-DMT salt is not administered in a crystalline form.

In an embodiment, the 5-MeO-DMT is not administered as a crystalline form of the benzoate salt. Crystalline forms of the benzoate salt are disclosed in WO2021250434 and are incorporated herein by reference. Crystalline forms of the hydrochloride salt are also disclosed in WO2021250434 and are incorporated herein by reference. In an embodiment, crystalline 5-MeO-DMT hydrochloride is characterised by peaks in an X-ray powder diffraction (XRPD) diffractogram at 2θ values of 9.2°±0.1°, 12.2°±0.1°, 14.1°±0.1°, 15.0°±0.1°, 18.5°±0.1°, and 19.5°±0.1°, as measured using an X-ray wavelength of 1.5406 Å.

In an embodiment, the salt anion is an aryl carboxylate. In an embodiment, the aryl carboxylate is substituted with one to three R groups. In an embodiment the one or more R groups are independently selected from: alkynyl, carbonyl, aldehyde, halo formyl, alkyl, halide, hydroxy, alkoxy, carbonate ester, carboxylate, carboxyl, carboalkoxy, methoxy, hydroperoxy, peroxy, ether, hemiacetal, hemiketal, acetal, ketal, orthoester, methylenedioxy, orthocarbonate ester, carboxylic anhydride, carboxamide, secondary, tertiary or quaternary amine, primary or secondary ketimine, primary or secondary aldimine, imide, azide, azo, cyanate, isocyanate, nitrate, nitrile, isonitrile, nitrosooxy, nitro, nitroso, oxime, pyridyl, carbamate, sulfhydryl, sulphide, disulfide, sulfinyl, sulfonyl, sulfino, sulfo, thiocyanate, isothiocyanate, carbonothioyl, carbothioic S-acid, carbothioic O-acid, thioester, thionoester, carbodithioic acid, carbodithio, phosphino, phosphono, phosphate, borono, boronate, borino or borinate. In an embodiment the one or more R groups are independently selected from: C₁-C₆ alkyl, C₁-C₆ alkoxy, C₁-C₆ alkenyl or C₁-C₆ alkynyl, and where each of these may be optionally substituted with one to three R groups as previously described.

In an embodiment, the 5-MeO-DMT or the pharmaceutical composition/formulation comprising 5-MeO-DMT and one or more pharmaceutically acceptable carriers or excipients, is for use in a method of one or more of: treating mental disorders, in particular treatment resistant depression, major depressive disorder, persistent depressive disorder, alcohol use disorder, anxiety disorder, post-traumatic stress disorder (PTSD), body dysmorphic disorder, obsessive-compulsive disorder, eating disorder and psychoactive substance abuse.

In an embodiment, the disease or condition is: conditions caused by dysfunctions of the central nervous system, conditions caused by dysfunctions of the peripheral nervous system, conditions benefiting from sleep regulation (such as insomnia), conditions benefiting from analgesics (such as chronic pain), migraines, trigeminal autonomic cephalgias (such as short-lasting unilateral neuralgiform headache with conjunctival injection and tearing (SUNCT), and short-lasting neuralgiform headaches with cranial autonomic symptoms (SUNA)), conditions benefiting from neurogenesis (such as stroke, traumatic brain injury, Parkinson's dementia), conditions benefiting from anti-inflammatory treatment, depression, treatment resistant depression, anxiety, substance use disorder, addictive disorder, gambling disorder, eating disorders, obsessive-compulsive disorders, or body dysmorphic disorders, optionally the condition is SUNCT and/or SUNA, alcohol-related diseases and disorders, eating disorders, impulse control disorders, nicotine-related disorders, tobacco-related disorders, methamphetamine-related disorders, amphetamine-related disorders, cannabis-related disorders, cocaine-related disorders, hallucinogen use disorders, inhalant-related disorders, benzodiazepine abuse or dependence related disorders, opioid-related disorders, tobacco addiction, alcohol abuse and/or addiction.

As used herein, “Clinical response” and/or “clinically significant reduction” and/or “clinically significant response” includes, but is not limited to, improvements on rating scales such as the Clinical Global Impression-Severity scale (CGI-S), the Patient Global Impression-Severity scale (PGI-S), the Clinical Global Impression-Improvement scale (CGI-I) or the Patient Global Impression-Improvement scale (PGI-I) and further includes, but is not limited to, endpoints such as the Montgomery-Asberg depression/major depressive disorder Rating Scale (MADRS) or the 17-item Hamilton depression/major depressive disorder Rating Scale (HAM-D) for depression/major depressive disorder and persistent depressive disorder, anxiety symptoms e.g. as measured by the Beck Anxiety Inventory (BAI), the Hamilton Anxiety Scale (HAM-A) or the State-Trait Anxiety Inventory (STAI) for anxiety disorder, the Clinician-Administered Posttraumatic Stress Disorder Scale (CAPS) for posttraumatic stress disorder, the Yale-Brown Obsessive Compulsive Scale Modified for Body Dysmorphic Disorder (BDD-YBOCS) for body dysmorphic disorder, the Yale-Brown Obsessive Compulsive Scale (Y-BOCS) for obsessive-compulsive disorder, weight gain for anorexia nervosa, frequency of binge-purge episodes for bulimia nervosa, frequency of binge episodes for binge eating disorder, duration of abstinence or reduced substance use in psychoactive substance abuse and suicidality rating scales such as the Columbia-Suicide Severity Rating Scale (C-SSRS) or the suicidal thoughts item of the MADRS for suicidal ideation or the Clinical Global Impression-Severity of Suicidality—Revised (CGI—SS-R) scale (the CGI—SS—R is derived from the CGI-S, and is scored 0=Normal, Not At All Suicidal; 1=Questionably Suicidal; 2=Mildly Suicidal; 3=Moderately Suicidal; 4=Markedly Suicidal; 5=Severely Suicidal; 6=Extremely Suicidal). When assessing a clinical response at an early time point after drug administration (e.g. at 2 hours) based on endpoints which have been developed for a longer recall period, a rational modification of such endpoint (e.g. changing the MADRS recall period to 2 hours and carrying forward the sleep item recorded at baseline before drug administration) may be applied.

The severity of a condition as well as changes of the severity can be assessed by the Clinical Global Impression (CGI) rating scales which are measures of symptom severity, treatment response and the efficacy of treatments. The CGI rating scales were developed to provide a brief, stand-alone assessment of the clinician's view of the patient's global functioning prior to and after a treatment (Busner, J. and Tagrum, S. D., 2007. The Clinical Global Impressions Scale: Applying a Research Tool in Clinical Practice. Psychiatry 2007, 29-37).

The CGI-Severity (CGI-S) is based on one question the clinician has to answer: “Considering your total clinical experience with this particular population, how mentally ill is the patient at this time?” This is rated on the following seven-point scale: 1=normal, not at all ill; 2=borderline mentally ill; 3=mildly ill; 4=moderately ill; 5=markedly ill; 6=severely ill; 7=among the most extremely ill patients. The CGI-S can be used to assess treatment success by comparing scores before and after treatment.

A clinical response may be reflected by a reduction in the Clinical Global Impression-Severity (CGI-S) score. According to the invention, a reduction in the CGI-S score means that the CGI-S is reduced by at least 1. Preferably, the CGI-S is reduced by at least 2 and/or to a score of 0. It is especially preferred if the CGI-S is reduced by at least 3 and/or to a score of 0. Alternatively, treatment success can be assessed using the CGI-Improvement (CGI-I), which is similarly simple in its format. After the treatment, the clinician compares the patient's overall clinical condition to the one prior to the treatment (the so-called baseline value). Again, only one query is rated on a seven-point scale: “Compared to the patient's condition at admission to the project [prior to medication initiation], this patient's condition is: 1=very much improved since the initiation of treatment; 2=much improved; 3=minimally improved; 4=no change from baseline (the initiation of treatment); 5=minimally worse; 6=much worse; 7=very much worse since the initiation of treatment.” The Patient Global Impression scale (PGI), also known as Subject Global Impression (SGI), is the counterpart to the Clinical Global Impressions scale (CGI). It consists of one item based on the CGI and adapted to the patient. It can measure disease severity (PGI-S) or disease improvement (PGI-I). Individual items of scales as described herein as well as sub-combinations of individual items may be used to assess specific disease aspects.

Numerous scales have been suggested to assess severity of one or more conditions or disorders, such as one or more conditions or disorders, such as one or more conditions or disorders, such as a mental disorder or a nervous system disorder. Such scales are based on tests which may be self-administered or administered by a clinician/physician. Scales which may be used according to the invention include those known in the art for diagnosis and/or monitoring the one or more conditions or disorders, such as a mental disorder or a nervous system disorder are discussed in more detail herein. Treatment outcome is assessed by using one or more indices or scales at one or more time points after completion of a treatment course.

The assessment may be carried out after the complete mystical experience has subsided. An appropriate point in time for an early assessment is generally about 2 to 3 hours after the last administration. An early assessment may generally be carried out, for instance, about 2 hours or about 3 hours after the last administration. An assessment of an effect on, for example, sleep disturbance can, however, be carried out at the earliest on the day after the treatment (i.e., on day 1) so that the treated patient/subject had the opportunity to sleep for at least one night.

Thus, an assessment at day 1 or on day 1 means an assessment on the day following the administration. The assessment may be carried out not earlier than 12 hours after the last administration and in any event optionally not earlier than one night after the last administration and not later than 36 hours after the last administration. The assessment may be carried out after about 24 hours. An assessment at day 7 or on day 7 means an assessment on the seventh day following the administration (the day of administration is day 0). Analogous definitions apply for other assessment timings measured in days.

When assessing a clinical response, for instance, using one of the scales to assess severity of one or more conditions or disorders, such as a mental disorder or a nervous system disorder, at an early time point after drug administration (e.g. at 1, 2 or 3 hours) based on endpoints which have been developed for a longer recall period (e.g. normally 7 days for the MADRS), a rational modification of such endpoint (e.g. changing the MADRS recall period to 1, 2 or 3 hours and carrying forward the sleep item recorded at baseline before drug administration) may be applied. The same applies with respect to any other scale applied herein, unless a recall period is specifically indicated. The considerations outlined apply for early time points because, on the one hand, in order to assess a clinical response, the influence of the patient's status before the treatment on any score recorded after treatment should be kept as low as possible, whereas on the other hand the sleep item cannot be assessed 1, 2 or 3 hours after drug administration. At later time points, for instance, on day 1 or later, typically all items of the relevant scales to assess a clinical response may be assessed, using, if necessary, an adapted recall period, so that it is not necessary to carry forward any pre-treatment score.

There are two fundamental types of sleep: rapid eye movement (REM) sleep and non-REM sleep. Non-REM sleep may be divided into four stages (I-IV). These non-REM stages correspond to an increasing depth of sleep. Non-REM and REM sleep alternate during each of the four to five cycles of normal human sleep each night. During the earlier proportion of the night, non-REM sleep is deeper and occupies a disproportionately large amount of time, particularly within the first cycle of sleep. As the night progresses, non-REM sleep becomes shallow and more of each cycle is allocated to REM sleep.

Normal healthy sleep consists of different phases as outlined above that proceed in successive, tightly regulated order through the night. Disruption of this tight regulation results in sleep disturbances. Sleep disturbance refers to conditions, whether idiopathic or occurring in the context of a medical condition such as for example one or more conditions or disorders, such as one or more conditions or disorders, such as a mental disorder or a nervous system disorder, that affect sleep quality, timing, or duration. It impacts a person's ability to properly function while the person is awake.

Common forms of sleep disturbances encompass disorders of initiating and maintaining sleep (insomnia), disorders of excessive somnolence (hypersomnia), disorders of sleep wake schedule (circadian rhythm disorders), dysfunctions associated with sleep, sleep stages, or partial arousals (parasomnia), disorders characterised by respiratory disturbance during sleep (sleep-related breathing disorders) and disorders characterised by abnormal movements during sleep (sleep-related movement disorders). Insomnia is a sleep disturbance where people have difficulty falling or staying asleep. People with insomnia have difficulty falling asleep; wake up often during the night and have trouble going back to sleep; wake up too early in the morning; have unrefreshing sleep; and/or have at least one daytime problem such as fatigue, sleepiness, problems with mood, concentration, accidents at work or while driving, etc. due to poor sleep.

Hypersomnia is characterised by excessive daytime sleepiness, and/or prolonged nighttime sleep. Sleep drunkenness is also a symptom found in hypersomnia patients/subjects. It is a difficulty transitioning from sleep to wake. Individuals experiencing sleep drunkenness report waking with confusion, disorientation, slowness and repeated returns to sleep.

Circadian rhythm disorders are characterised by chronic or recurring sleep disturbances due to alterations of the individual's internal circadian rhythm or due to misalignments between their circadian rhythm and their desired or required work or social schedule. This dyssynchrony may be transient or persistent. The ensuing clinical picture combines elements of both insomnia and hypersomnia. Sleep periods are usually shortened and disrupted, performance during the desired waking state is impaired, and temporary opportunities to revert to a regular sleep schedule are unsuccessful.

Parasomnia designates various forms of sleep disturbance characterised by abnormal behavioural or physiological activity (such as sleepwalking or nightmares) that people experience prior to falling asleep, while asleep, or during the arousal period between sleep and wakefulness. There are considerable variations in terms of characteristics, severity, and frequency. Parasomnia may compromise the quality of sleep.

Sleep-related breathing disorders are characterised by abnormal and difficult respiration during sleep. Respiration is a complex process that relies heavily on the coordinated action of the muscles of respiration and the (control centre in the) brain. One form of a sleep-related breathing disorder is central sleep apnoea. It occurs when the brain stops sending signals that control breathing, for instance, based on an underlying health condition. Central sleep apnoea has a potentially serious impact on sleep and the balance of oxygen and carbon dioxide in the blood. The reduction of airflow leads to intermittent hypoxia which in leads to sleep fragmentation due to microarousals or awakenings. A consequence may be excessive daytime sleepiness.

In sleep-related movement disorders repetitive, relatively simple, usually stereotyped, movements interfere with sleep or its onset. The most common of these are restless leg syndrome (RLS) and periodic limb movement disorder (PLMD). Not getting the proper amount or quality of sleep may lead to personality changes and may not only exacerbate existing mental illness, but also be a trigger for the development of mental illness. Sleep disturbance may also interfere with cognitive function and lead to memory impairment. A patient/subject who is deprived of sleep may experience difficulty making decisions, irritability, have problems with performance, and may have slower reaction times. Sleep loss may also adversely affect life by contributing to the development of obesity, diabetes, and heart disease.

Treatment of sleep disorders varies depending on the type and underlying cause. Maintenance of good sleep hygiene, a healthy sleep environment, and a consistent sleep-wake schedule are often considered as first-line treatment. If not successful, treatment also involves pharmacotherapy or psychotherapy. Available treatments are not successful in all patients/subjects, may be associated with side effects and/or require treatment over a long period of time to achieve a relevant treatment effect. In patients/subjects suffering from sleep disturbance in association with one or more conditions or disorders, such as a mental disorder or a nervous system disorder known treatments of the mental or nervous system disorder do not necessarily improve the sleep disturbance.

For instance, sleep disturbance is frequently associated with mental disorders, such as depression. However, treatment of depression does not necessarily lead to an improvement of the concomitant sleep disturbances. While most antidepressants have been proven to influence the sleep architecture, some classes of antidepressants improve sleep, but others may cause sleep impairment. Sleep may be assessed by measuring parameters such as sleep duration, sleep architecture, sleep latency, and the frequency and duration of awakenings throughout the night. The quantitative metrics may be measured using objective methods, including polysomnography, actigraphy, and the determination of sleep latency, or by way of self reported measures (questionnaires).

Polysomnography is a technique requiring that a patient/subject is monitored overnight at a specialised clinic. A variety of functions are measured throughout the night, including eye movements, brain and muscle activity, respiratory effort and airflow, blood oxygen levels, body positioning and movements, snoring, and heart rate. Another quantitative measurement is actigraphy. An actimetry sensor is worn to measure motor activity, which is recorded continually and used to assess sleep-wake cycles. This technique allows the patient/subject to continue normal routines while the required data are being recorded in a natural sleep environment.

Sleep latency may be measured by the multiple sleep latency test (MSLT). This test provides an objective measure to determine how long it takes a person to fall asleep across a multiplicity of test naps. An average sleep latency of approximately 10 minutes is considered to be normal; less than eight minutes is indicative of sleep disturbance (excessive daytime sleepiness). Accompanying analysis of brain activity may assist in the further diagnosis of the sleep disturbance.

Sleep-rating questionnaires capture ratings of components of sleep quality, such as perceptions of sleep depth, rousing difficulties, and restfulness after sleep, in addition to other factors that could affect sleep quality, such as comorbid conditions and medication use. The evaluation of the qualitative aspects of sleep experience is important, as sleep complaints may often persist despite normal values for quantitative measures of sleep. Questionnaires not only facilitate a quick and accurate assessment of a complex clinical problem, but they are potentially also helpful for tracking a patient's progress.

Various sleep quality indexes are known. The following indexes include examples of questionnaires to assess sleep in general and questionnaires to assess in particular insomnia, hypersomnia, circadian rhythm disorders and parasomnia, respectively. The invention is, however, not limited to the use of a particular index or questionnaire.

Some questionnaires rely on recall periods (recall windows) of several days or even weeks. While this may be appropriate for diagnosing sleep disturbances, it is not always appropriate for assessing treatment effects, in particular rapid onset of effect after treatment. For several of the questionnaires the recall period may be modified so that the scores obtained reflect a period after treatment. Questionnaires specifically discussed herein to assess effects of a treatment on sleep in patients/subjects suffering from specific conditions rely on a recall period that does not start earlier than the time point when complete mystical experiences have subsided after the last administration. To meet this criterion, the normally applied recall period is modified if necessary.

Sleep quality in general may be assessed, for instance, with the Sleep-50 questionnaire. The SLEEP-50 questionnaire consists of 50 items designed to screen for a variety of sleep disorders in the general population. The scale consists of nine subscales, reflecting some of the most common disorders and complaints related to sleep and the factors required for diagnosis such as sleep apnoea, insomnia, narcolepsy, restless legs/periodic leg movement disorder, circadian rhythm sleep disorder, sleepwalking, nightmares, factors influencing sleep, and the impact of sleep complaints on daily functioning. For each item, respondents are provided with a scale ranging from 1 (“not at all”) to 4 (“very much”) and are asked to indicate the extent to which the statement has matched their experience over the previous month or another appropriate recall window.

For diagnosing a sleep disorder not only the specific subscale (e.g., insomnia) must exceed a certain cut-off point, but respondents must also meet a cut-off of at least 3 or 4 (“rather much” or “very much”, respectively) on the subscale evaluating the impact of sleep complaints on daily functioning.

Treatment success may be indicated (i) by a decrease of the score, preferably (ii) by a decrease to below the cut-off value. A common questionnaire assessing sleep disturbance is the Pittsburgh Sleep Quality Index. Other instruments are the insomnia severity index, the Espie sleep disturbance questionnaire and the patient/subject Reported Outcomes Measurement Information System (PROMIS®) Sleep Disturbance.

The Pittsburgh Sleep Quality Index (PSQI) assesses overall sleep quality and disturbances. The PSQI is a self-rated questionnaire comprising 19 questions. Respondents are asked to indicate how frequently they have experienced certain sleep difficulties over the past month or another appropriate recall window. The 19 self-rated questions assess a wide variety of factors relating to sleep quality, including estimates of sleep duration and latency and of the frequency and severity of specific sleep-related problems. These 19 items are grouped into seven component scores: (1) patient/subjective sleep quality; (2) sleep latency; (3) sleep duration; (4) habitual sleep efficiency; (5) sleep disturbances; (6) use of sleeping medication; (7) daytime dysfunction. Each component is assigned a score of 0 to 3. Higher scores indicate more acute sleep disturbances.

The seven component scores are then summed to yield one global score, with a range of 0-21 points, “0” indicating no difficulty and “21” indicating severe difficulties in all areas. A global score cut-off of 5 distinguishes poor from good sleepers. A global score >5 indicates that a patient/subject is having severe difficulties in at least two areas, or moderate difficulties in more than three areas. If treatment outcome is assessed using the PSQI, treatment success may be indicated (i) by a decrease of the score, preferably (ii) by a decrease to 5 or below.

The insomnia severity index (ISI) is a short questionnaire relating to patient/subjective sleep quality, severity of symptoms, patient/subjective satisfaction with sleep, the degree to which insomnia interferes with daily functioning, how noticeable the respondent feels his or her insomnia is compared to others, and the overall level of distress created by the sleep problem. Individual responses may be scored from 0 (=none) to 4 (=very); a higher total score corresponds to more severe insomnia. A total score of 0-7 indicates “no clinically significant insomnia,” 8-14 means “subthreshold insomnia,” 15-21 is “clinical insomnia (moderate severity),” and 22-28 means “clinical insomnia (severe)”. The recall window is two weeks. Another appropriate recall window may also be used. Treatment success may be indicated (i) by a decrease of the score, for instance, by >7 points, in particular >8 point; preferably (ii) by a decrease to below the cut-off value for clinically significant insomnia.

The Espie sleep disturbance questionnaire (SDQ) evaluates patient/subjective experiences of insomnia. With ratings on restlessness/agitation, mental overactivity, consequences of insomnia, and lack of sleep readiness, the SDQ is concerned specifically with beliefs about the sources of sleep issues. Respondents use a five-point scale to indicate how often certain statements about insomnia are representative of their experience. 1 means “never true,” while 5 means “very often true.” Higher scores are indicative of more dysfunctional beliefs about the causes and correlates of insomnia.

Treatment success may be indicated by a decrease of the score. The Patient-Reported Outcomes Information System (PROMIS)® Sleep Disturbance instrument is a universal measure to evaluate sleep disturbances. The instrument is available as a long form and 4 different short forms (e.g., 4-, 6-, and 8-items) and assesses self-reported perceptions of sleep quality, sleep depth, and any perceived difficulties related to getting and staying asleep over a 7-day period. Each item on the measure is rated on a 5-point scale. The raw scores on the items are summed to obtain a total raw score. Total raw scores are then converted into a standardised T-score using conversion tables. Treatment success may be indicated by a decrease of the T-score.

Hypersomnia or hypersomnolence may be assessed by the Epworth Sleepiness Scale, the Stanford Sleepiness Scale, or the Idiopathic Hypersomnia Severity scale. The Epworth Sleepiness Scale (ESS) evaluates overall daytime sleepiness. The questionnaire asks respondents to rate how likely they are to fall asleep in eight different situations representing a moment of relative inactivity, such as a nap in the afternoon or sitting in a car stopped in traffic. Using a scale of 0-3 (with 0 meaning “would never doze” and 3 meaning “high chance of dozing”), respondents rate their likelihood of falling asleep. Scoring ranges from 0-24; the higher the score, the higher the severity of daytime sleepiness. A cut-off score of 10 identifies daytime sleepiness at a potentially clinical level. Treatment success may be indicated (i) by a decrease of the score, preferably (ii) by a decrease to 10 or below.

The Stanford Sleepiness Scale is a patient/subjective measure of sleepiness, evaluating sleepiness at specific moments in time. Consisting of only one item, the scale requires respondents to select one of seven statements best representing their current level of perceived sleepiness. A scale from 1 (=Feeling active and vital; alert; wide awake) to 7 (=Almost in reverie; sleep onset soon; lost struggle to remain awake) is used to assess the level of sleepiness. Treatment success may be indicated by a decrease of the score.

Parasomnias may be evaluated by the Paris Arousal Disorders Severity Scale (PADSS). The Paris Arousal Disorders Severity Scale (PADSS) is a self-rating scale listing parasomniac behaviours, assessing their frequency and includes an evaluation of consequences. Treatment success may be indicated (i) by a decrease of the score, preferably (ii) by a decrease to below the cut-off value.

A common questionnaire that assesses sleep-related breathing disorders is the Berlin Questionnaire. An appropriate recall period may also be chosen. Treatment success may be indicated by a decrease of the score. A common questionnaire that assesses sleep-related movement disorders is the International Restless Legs Syndrome Study Group Rating Scale. The 10-item questionnaire asks respondents to use Likert-type ratings to indicate how acutely the disorder has affected them over the course of the past week. Questions may be divided into one of two categories: disorder symptoms (nature, intensity, and frequency) and their impact (sleep issues, disturbances in daily functioning, and resultant changes in mood. Each of the ten questions requires respondents to rate their experiences with RLS on a scale from 0 to 4, with 4 representing the most severe and frequent symptoms and 0 representing the least. Total scores may range from 0 to 40. As a brief scale with excellent psychometric qualities, the instrument may be suitable for a variety of research and clinical purposes, including screening and assessment of treatment outcomes. Treatment response may be assessed by a decrease of the score

Bipolar disorder (BD) has various aspects and is characterised by various symptoms. The predominant psychopathology is depression, and the presentation of a patient/subject experiencing a depressive phase may initially result in the diagnosis of that patient/subject as having major depressive disorder (MDD). However, BD possesses multiple characteristics that define it as distinct from the latter even during the depressive phase.

Of particular interest here are the symptoms that are more strongly associated with BD compared to other psychiatric disorders, as these are the metrics against which patient/subject treatment is assessed. Notwithstanding that many symptoms may be said to straddle multiple disorders, much work has been done to identify several symptoms that present strongly in BD patients/subjects: sleep disturbance, psychomotor retardation (reduced energy and activity and reduced motivation), negative thinking (feelings of worthlessness; helplessness and hopelessness; guilt), anxiety, cognitive dysfunction (impaired concentration and memory) and social/emotional withdrawal or detachment (anhedonia, emotional withdrawal and affective flattening). Characteristic symptoms further include suicidal ideation. Still further, characteristic symptoms include mixed symptoms (psychotic symptoms; irritability; lability; increased motor drive; increased speech; agitation).

Clinical assessment tools such as the Bipolar Depression Rating Scale (BDRS) have been developed and validated for use in BD, which take into account these symptoms. The Bipolar Depression Rating Scale (BDRS) is designed to measure the severity of depressive symptoms in bipolar depression. The BDRS is validated for clinical use by trained raters. Based on a clinical interview, the BDRS items rate the severity of depressive and/or mixed symptoms expressed by patients/subjects currently and during the past few days. If there is a discordance between symptoms currently and the last few days, the rating should reflect current symptoms. The scale contains 20 questions and the maximum score possible is 60. Higher scores indicate greater severity.

The questions address depressed mood; sleep disturbance; appetite disturbance; reduced social engagement; reduced energy and activity; reduced motivation; impaired concentration and memory; anxiety; anhedonia; affective flattening; feelings of worthlessness; feelings of helplessness and hopelessness; suicidal ideation; feelings of guilt; psychotic symptoms; irritability; lability; increased motor drive; increased speech; agitation. Each of these aspects is assessed and assigned a score of 0, 1, 2 or 3.

Depressed mood is scored as 0 if there is no self-reported and/or observed depression as evidenced by gloom, sadness, pessimism, hopelessness, and helplessness; 1 (mild) in case of brief or transient periods of depression, or mildly depressed mood; 2 (moderate) in case a depressed mood is clearly but not consistently present and other emotions are expressed, or depression is of moderate intensity; 3 (severe) in case of pervasive or continuous depressed mood of marked intensity.

Sleep disturbance (sleep dysregulation) is assessed based on the change in total amount of sleep over a 24-hour cycle, rated independent of the effect of external factors. It may either take the form of insomnia (reduction in total sleep time) or the form of hypersomnia (increase in total sleep time, inclusive of daytime sleep). The rating for insomnia involves scores of 0 (no reduction in total sleep time); 1 (mild; reduction up to 2 hours); 2 (moderate; 2-4 hours); 3 (severe; more than 4 hours).

The alternative rating for hypersomnia involves scores of 0 (no increase in total sleep time, inclusive of daytime sleep); 1 (mild; less than 2 hours, or normal amount but nonrestorative); 2 (moderate; 2-4 hours); 3 (severe; greater than 4 hours).

Appetite disturbance is assessed based on the change in appetite and food consumption, rated independent of the effect of external factors. It may either take the form of loss of appetite or the form of increase in appetite. The rating for loss of appetite involves scores of 0 (no change in appetite and food consumption); 1 (mild; no change in food intake, but has to push self to eat or reports that food has lost taste); 2 (moderate; some decrease in food intake); 3 (marked decrease in food intake, hardly eating). The alternative rating for increase in appetite involves scores of 0 (no change in appetite and food consumption); 1 (mild; no change in food intake, but increased hunger); 2 (moderate; some increase in food intake, e.g., comfort eating); 3 (marked increase in food intake or cravings).

Reduced social engagement is scored as 0 if there are no patient/subjective reports of reduced social and interpersonal engagement or interactions; 1 (mild) in case of slight reduction in social engagement with no impairment in social or interpersonal function; 2 (moderate) in case of clear reduction in social engagement with some functional sequelae, e.g., avoiding some social engagements or conversations; and 3 (severe) in case of marked reduction in social interaction or avoidance of almost all forms of social contact, e.g., refusing to answer the phone or see friends or family.

Reduced energy and activity is scored as 0 if there is no reduced energy, drive or goal directed behaviour; 1 (mild) in case of ability to engage in usual activities but with increased effort; 2 (moderate) in case of significant reduction in energy leading to reduction of some role-specific activities; and 3 (severe) in case of leaden paralysis or cessation of almost all role specific activities, (e.g., spending excessive time in bed, avoiding answering the phone, poor personal hygiene).

Reduced motivation is scored as 0 if there are no reports of patient/subjective reduction in drive, motivation, and consequent goal directed activity; 1 (mild) in case of a slight reduction in motivation with no reduction in function; 2 (moderate) in case of a reduced motivation or drive with significantly reduced volitional activity or requiring substantial effort to maintain usual level of function; and 3 (severe) in case of reduced motivation or drive such that goal directed behaviour or function is markedly reduced.

Impaired concentration and memory are scored as 0 if there are no patient/subjective reports of reduced attention, concentration, or memory, and consequent functional impairment; 1 (mild) in case of slight impairment of attention, concentration, or memory with no functional impairment; 2 (moderate) in case of significant impairment of attention, concentration, or forgetfulness with some functional impairment; 3 (severe) in case of marked impairment of concentration or memory with substantial functional impairment, e.g., unable to read or watch TV).

Anxiety is scored as 0 if there are no patient/subjective reports of worry, tension, and/or somatic anxiety symptoms e.g., tremor, palpitations, dizziness, light-headedness, pins and needles, sweating, dyspnoea, butterflies in the stomach, or diarrhoea; 1 (mild; transient worry or tension about minor matters); 2 (moderate; significant anxiety, tension, or worry, or some accompanying somatic features); 3 (severe; marked continuous anxiety, tension, or worry that interferes with normal activity; or panic attacks). Anhedonia is scored as 0 (no patient/subjectively reduced ability to experience pleasure in usual activities); 1 (mild; slight reduction in pleasure from usually pleasurable activities); 2 (moderate; significant reduction in pleasure from usually pleasurable activities; some pleasure from isolated activities retained); or 3 (severe; complete inability to experience pleasure). Affective flattening is scored as 0 if there is no patient/subjective sense of reduced intensity or range of feelings or emotions; 1 (mild) in case of slight constriction of range of affect, or transient reduction in range or intensity of feelings; 2 (moderate) in case of significant constriction of range or intensity of feelings with preservation of some emotions, e.g., inability to cry; and 3 (severe) in case of marked and pervasive constriction of range of affect or inability to experience usual emotions.

Feelings of worthlessness (also simply referred to as worthlessness) are scored as 0 (no patient/subjective sense, or thoughts, of decreased self-value or self-worth); 1 (mild; slight decrease in sense of self-worth); 2 (moderate; some thoughts of worthlessness and decreased self-worth) 3 (severe; marked, pervasive, or persistent feelings of worthlessness, e.g., feels others better off without them, unable to appreciate positive attributes).

Feelings of helplessness and hopelessness (also simply referred to as helplessness and hopelessness) characterise the patient/subjective sense of pessimism or gloom regarding the future, inability to cope, or sense of loss of control. If this is absent, the score is 0. The score is 1 (mild) in case of occasional and mild feelings of not being able to cope as usual, or pessimism; it is 2 (moderate) in case the patient/subject often feels unable to cope, or has significant feelings of helplessness or hopelessness which lift at times; it is 3 (severe) if there are marked and persistent feelings of pessimism, helplessness, or hopelessness.

Suicidal ideation relates to thoughts or feelings that life is not worthwhile; thoughts of death or suicide and is scored 0 if such thoughts are absent; 1 (mild) in case of thoughts that life is not worthwhile or is meaningless; 2 (moderate) in case of thoughts of dying or death, but with no active suicide thoughts or plans; 3 (severe) in case of thoughts or plans of suicide. Feelings of guilt (also simply referred to as guilt) are scored as 0 if there is no patient/subjective sense of self blame, failure, or remorse for real or imagined past errors; 1 (mild) in case of slight decrease in self-esteem or increased self-criticism; 2 (moderate) in case of significant thoughts of failure, self-criticism, inability to cope, or ruminations regarding past failures and the effect on others; able to recognise as excessive; 3 (severe) in case of marked, pervasive, or persistent guilt, e.g., feelings of deserving punishment; or does not clearly recognise as excessive.

Psychotic symptoms are scored as 0 if overvalued ideas, delusions, or hallucinations are absent; 1 (mild) in case of mild overvalued ideas, e.g., self-criticism or pessimism without clear effect on behaviour; 2 (moderate) in case of significant overvalued ideas with clear effect on behaviour, e.g., strong guilt feelings, clear thoughts that others would be better off without them; 3 (severe) in case of clear psychotic symptoms, e.g., delusions or hallucinations.

Irritability reports uncharacteristic patient/subjective irritability, short fuse, easily angered, manifested by verbal or physical outbursts and is scored 0 if absent; 1 (mild) in case of slight patient/subjective irritability which may not be overtly present; 2 (moderate) in case of verbal snappiness and irritability that is clearly observable in the interview; 3 (severe) in case of reports of physical outbursts, e.g., throwing/breaking objects, or markedly abusive verbal outbursts. Lability is scored 0 if there are no observed mood lability or reported mood swings. It is scored 1 (mild) in case of patient/subjective reports of mild increase in mood lability; 2 (moderate) if mood lability is clearly observable, moderate in intensity; 3 (severe) in case of marked and dominant mood lability, frequent or dramatic swings in mood.

Increased motor drive relates to patient/subjective reports and objective evidence of increased motor drive and motor activity. It is scored 0 in case of normal motor drive: 1 (mild) in case of a slight increase in drive, not observable in the interview; 2 (moderate) in case of clear and observable increase in energy and drive; 3 (severe) if there is a marked or continuous increase in drive. Increased speech relates to an observed increase in either the rate or quantity of speech, or observed flight of ideas. This item is scored 0 if such observations are absent; 1 (mild) if there is a slight increase in the rate or quantity of speech; 2 (moderate) in case of racing thoughts, or if the patient/subject is significantly more talkative, clearly distractible, or in case of some circumstantiality; wherein this does not impede the interview; 3 (severe) in case of flight of ideas; which interferes with the interview.

Agitation is scored 0 if there is no observed restlessness or agitation; 1 (mild) in case of slight restlessness; 2 (moderate) in case of clear increase in level of agitation; 3 (severe) in case of marked agitation, e.g., near continuous pacing or wringing hands. While a higher score on the BDRS scale indicates more severe disease, there are no generally accepted limits for when a patient/subject is to be considered moderately or severely ill. BDRS score ranges used herein for indicating the severity of depressive episodes in patients/subjects with bipolar disorder are 13-18 for “mildly ill”, 19-23 for “moderately ill”, 24-36 for “markedly ill”, 37-39 for “severely ill”, and *40 for “extremely ill”. Various other scales are also useful to assess the severity of disease as well as the clinical outcome of treatments.

Anxiety is sometimes defined as an “apprehensive anticipation of future danger or misfortune accompanied by a feeling of dysphoria or somatic symptoms of tension”. Anxiety is characterised by an intense, excessive, and persistent worry and fear about a situation that is only patient/subjectively seen as menacing and is often accompanied by muscular tension, restlessness, fatigue, inability to catch one's breath, tightness in the abdominal region, nausea, and problems in concentration.

In anxiety disorders or other mental or nervous system disorders associated with anxiety, the feelings of anxiety are difficult to control and interfere with daily activities. Anxiety is a core feature of anxiety disorders, including separation anxiety disorder, specific phobia, social anxiety disorder (social phobia), panic disorder, generalised anxiety disorder (GAD), agoraphobia, and substance/medication-induced anxiety disorder. Anxiety is moreover associated with several other mental and nervous system disorders. Anxiety is also associated with sleep disturbance.

Several rating scales to assess anxiety are known on the art, and anxiety symptoms are furthermore assessed as part of various rating scales used to assess mental and nervous system disorders. The Hamilton Anxiety Rating Scale (HAM-A) is designed to assess anxiety symptoms. The scale is clinician/physician-administered. It has 14 items which may be divided into a group of psychic items (1-6 and 14) measuring in particular mental agitation and psychological distress and into a group of somatic items (items 7-13) measuring in particular physical complaints related to anxiety. Each item is rated by the interviewer on a scale from 0 to 4:0=Not present, 1=Mild, 2=Moderate, 3=Severe, 4=Very severe. A total score is obtained by summing the 14 items. The total score range is 0-56. Higher scores indicate more anxiety. A score $7 is considered to represent no or minimal anxiety; a score of 8-14 mild anxiety; a score of 15-23 moderate anxiety; a score ≥24 severe anxiety.

The Beck Anxiety Inventory (BAI) is a 21-item self-report questionnaire developed to assess anxiety, with a focus on somatic symptoms. The items are rated on a four-point Likert scale ranging from zero (not at all) to three (severely: I could barely stand it). The total score ranges from 0 to 63. Subthreshold anxiety as the term is used herein in particular means that the patient/subject has a Hamilton Rating Scale for Anxiety (HAM-A) score of at least 9 but of less than 18 and/or a Beck Anxiety Inventory (BAI) score of at least 11 but of less than 16. Negative thinking or individual aspects thereof, such as worthlessness, helplessness and hopelessness, and guilt, may be evaluated by different instruments, such as questionnaires or scales.

Questionnaires assess the mental status of a patient/subject based on observations made by the patient/subject himself/herself, caregivers or the clinician/physician administering the questionnaire. Questionnaires used to assess whether a patient/subject suffers from a particular mental or nervous system disorder may comprise items related to negative thinking. Instruments evaluating relevant aspects of negative thinking include, for example, the State Shame and Guilt Scale (SSGS), the Positive and Negative Affect Schedule-Expanded Form (PANAS-X) or the State Hope Scale (SHS).

The State Shame and Guilt Scale (SSGS) is a self-rating scale of in-the-moment (state) feelings of shame, and guilt experiences. It comprises two subscales, a shame and a guilt subscale. The shame subscale comprises items 1, 3, 5, 7, 9. The guilt subscale comprises items 2, 4, 6, 8, 10. All items are scored in a positive direction and are rated on a 5-point Likert scale. It contains some statements which may or may not describe how the patient/subject is feeling right now. A higher score indicates a more intense feeling of shame or guilt. The Positive and Negative Affect Schedule-Expanded Form (PANAS-X) is a 60-item, expanded version of the PANAS. The PANAS-X measures 11 specific affects: Fear, Sadness, Guilt, Hostility, Shyness, Fatigue, Surprise, Joviality, Self-Assurance, Attentiveness, and Serenity. The PANAS-X thus provides for mood measurement at two different levels. The basic negative emotion scales are fear, hostility, guilt and sadness, while the scale of guilt encompasses six items: guilty, ashamed, blameworthy, angry at self, disgusted with self, dissatisfied with self. Each answer should be scored as 1=very slightly or not at all; 2=a little; 3=moderately; 4=quite a bit; or 5=extremely. However, investigators facing more severe time constraints may select and assess only those scales that are most relevant to their research.

More intense feelings of guilt are reflected by a higher score on the guilt scale. The PANAS-X is simple and easy to administer. Most patients/subjects complete the entire 60-item schedule in 10 minutes or less. This scale consists of a number of words and phrases that describe different feelings and emotions. While it should be indicated to what extent the patient/subject has felt this way during the past few weeks, it has been found that the trait scores on the PANAS-X scales are stable over time, including “at the present moment”, “today” and during “the past few days”, indicating that an appropriate shorter recall period may be applied.

The State Hope Scale (SHS) has three agency and three pathways items to which respondents describe themselves in terms of how they are “right now.” The agency subscale score is derived by summing items 2, 4 and 6, relating to the perceived capacity to use one's pathways to reach desired goals; the pathways subscale score is derived by adding the items 1, 3 and 5, relating to thinking that is used to identify possible ways to achieve a goal. The total State Hope Scale score is derived by summing the three agency and the three pathways items. Scores may range from a low of 6 to a high of 48, wherein higher hope is reflected by a higher score on this scale. Negative thinking or aspects thereof are also reflected in other scales such as HAM-D, the MADRS, the BPRS or the BDRS, wherein relevant items thereof may be commonly applicable for assessing negative thinking or aspects thereof.

Cognition includes the skills needed for thinking, remembering, paying attention, and solving problems. Loss or decline of these skills leads to cognitive dysfunction, a term used herein to refer to a deficit in, or an impairment of, any domain of cognition. Cognitive dysfunction may be one of the manifestations of a patient's underlying condition.

The DSM-5 defines six key domains of cognitive function, namely complex attention, executive function, learning and memory, language, perceptual-motor function, and social cognition. Cognitive dysfunction may impact one or more of those domains. In fact, cognitive abilities are highly interrelated, and it is not unusual that more than one domain is affected. For instance, the domain complex attention has the subdomains sustained attention (commonly referred to as ‘concentration’ or ‘focus’), divided attention, selective attention, and processing speed.

Thus, complex attention evidently encompasses aspects which are critical for a variety of cognitive tasks, such as executive function and learning and memory. Cognitive control or executive function is intrinsically attentional. Also, perception, and decision-making are profoundly influenced by attention abilities. As a consequence, attention is not only tested for in isolation, but for example, also tested by cognitive control tasks/executive function. If attention is impaired, other types of cognitive abilities will likely also be impaired. Before language may be comprehended, visual-spatial relationships perceived, information remembered or problems solved, the stimuli must be attended to.

Cognitive dysfunction, which term herein means an acquired condition and thus represents a decline from a previously attained level of functioning, may be associated with various processes. In a healthy individual, certain cognitive abilities, such as accumulated knowledge and vocabulary, are maintained upon ageing and may even improve over time. However, even in the absence of any pathological condition, ageing leads to declines in abilities like thinking abstractly, reasoning, and decision-making. These deteriorations are linked to underlying age-related deficits in processing speed, attention, memory, and executive function, which are indicative of cognitive ageing.

Independent of normal ageing, cognitive dysfunction may be associated with one or more conditions or disorders, such as a mental disorder or a nervous system disorder or some other medical conditions. Mental or nervous system disorders which lead to, or are associated with, cognitive dysfunction include disorders characterised by depressive episodes, for example, Major Depressive Disorder (MDD), Postpartum Depression (PPD), Persistent Depressive Disorder, Seasonal Affective Disorder and Bipolar Disorder (BD), such as Bipolar I Disorder and Bipolar II Disorder; Anxiety Disorder, for example Separation Anxiety Disorder, Agoraphobia, Generalised Anxiety Disorder (GAD), Social Anxiety Disorder (SAD), Panic Disorder, Phobia, and Substance/Medication Induced Anxiety Disorder; Somatic Symptom Disorder; Obsessive Compulsive and Related Disorders, for example, Obsessive Compulsive Disorder (OCD) and Body Dysmorphic Disorder (BDD); Post Traumatic Stress Disorder (PTSD); Pain Disorders, for example Chronic Pain, Fibromyalgia and Migraine; Mental and Behavioural Disorders due to Psychoactive Substance Use, for example, Substance Use Disorder (SUD); Psychotic Disorders, for example Schizophrenia; Huntington's Disease; Parkinson's Disease; Dementia, for example Alzheimer's Dementia (AD), Parkinson's Disease Dementia (PDD), Dementia with Lewy Bodies, Vascular Dementia, Frontotemporal Dementia; Eating Disorders; Attention Deficit Hyperactivity Disorder (ADHD); Personality Disorders, for example Schizotypal Personality Disorder and Borderline Personality Disorder; Chronic Fatigue Syndrome; one or more conditions or disorders, such as a mental disorder or a nervous system disorder associated with HIV, Traumatic Brain Injury or Post COVID Condition. The cognitive dysfunction may also occur in a patient/subject suffering from sleep disturbance, for instance, insomnia.

Cognitive dysfunction furthermore occurs in disorders showing symptoms characteristic of a neurocognitive disorder that cause clinically significant distress or impairment in social, occupational, or other important areas of functioning but do not meet the full criteria for any aetiology-related disorder. Cognitive dysfunction may take the form of a neurocognitive disorder. Mild neurocognitive disorder, also referred to as mild cognitive impairment, is characterised by a modest cognitive decline from a previous level of performance in one or more of the cognitive domains. Affected patients/subjects are still able to stay independent and do daily tasks. However, the patient/subject usually functions at a suboptimal level. Everyday tasks become more effortful owing to the engagement of compensatory strategies to maintain independence. In major neurocognitive disorder, a significant cognitive decline from a previous level of performance in one or more of the cognitive domains is observed. The cognitive deficits interfere with independence in everyday activities.

Cognitive dysfunction may be evaluated by questionnaires or by neuropsychological assessments. Questionnaires assess the mental status of a patient/subject based on observations made by the patient/subject himself, caregivers or the clinician/physician administering the questionnaire. Questionnaires used to assess whether a patient/subject suffers from a particular mental or nervous system disorder may comprise items related to cognitive function. A neuropsychological assessment is a process by which a person's cognitive, psychological/emotional and behavioural functioning is comprehensively evaluated. A core part of neuropsychological assessment is the administration of neuropsychological tests for the formal assessment of cognitive function.

Performance in these tests is compared with norms appropriate to the patient's age, educational attainment, and cultural background. Testing often uses a set of performance-based questions, also known as a neuropsychological test battery. The abilities tested include language processing, visuospatial processing, attention/concentration, verbal learning and memory, visual learning and memory, executive functions, speed of processing, and sensory-perceptual functions.

Common tests that assess cognitive dysfunction are the Montreal Cognitive Assessment (MoCA), the Mini-Mental State Examination (MMSE), the Mini-Cog™, the Screen for Cognitive Impairment in Psychiatry (SCIP), and the MATRICS Consensus Cognitive Battery (MCCB). The Montreal Cognitive Assessment (MoCA) is a widely used screening assessment for detecting cognitive impairment. It assesses different cognitive domains: short-term memory; visuospatial abilities; executive functions; attention, concentration and working memory; language; orientation to time and space. The total possible score is 30 points; a score of 26 or above is considered normal; a score of 18-25 is considered mild cognitive impairment, a score of 10-17 is considered moderate cognitive impairment and a score less than 10 is considered severe cognitive impairment.

The Mini-Mental State Examination (MMSE) is an 11-question measure that tests five areas of cognitive function: orientation, registration, attention and calculation, recall, and language. The maximum score is 30. The raw score may also need to be corrected for educational attainment and age. Four cut-off levels are employed herein to classify the severity of cognitive impairment: 24-30 means no cognitive impairment; 19-23 means mild cognitive impairment; 10-18 means moderate cognitive impairment; and *9 means severe cognitive impairment. Used repeatedly, the MMSE is suitable to measure changes in cognitive status. The Mini-Cog™ is a short cognitive impairment screening questionnaire. It combines a 3-word recall with a clock drawing test. The clock drawing test assesses many cognitive areas that may be affected, such as executive function, visuospatial abilities, motor programming, and attention. One point is given for each of the three words correctly recalled after performing the clock drawing test; a correctly drawn clock is worth two points. A score of <4 indicates cognitive impairment.

The Screen for Cognitive Impairment in Psychiatry (SCIP) is a well-evaluated screening instrument for the examination of cognitive performance in psychiatric patients/subjects. The SCIP consists of five subscales: verbal learning test-immediate (VLT-I), working memory test (WMT), verbal fluency test (VFT), verbal learning test-delayed (VLT-D) and processing speed test (PST). There are three different test forms to facilitate test repetition and therefore reducing learning effect. Subscale scores are calculated for each of the five tests, and a total score is calculated from the sum of the subscale scores. A total score of less than 70 indicates cognitive dysfunction.

Cognitive dysfunction may also be assessed by the MCCB (MATRICS Consensus Cognitive Battery) or by one or more of the various subtests. The subtests are: Trail Making Test, Part A (testing speed of processing); Brief Assessment of Cognition in Schizophrenia, symbol coding subtest (speed of processing); Hopkins Verbal Learning Test-Revised, immediate recall, three learning trials only (verbal learning); Wechsler Memory Scale, 3rd ed., spatial span subtest (working memory (nonverbal)); Letter-Number Span test (working memory (verbal)); Neuropsychological Assessment Battery, mazes subtest (reasoning and problem solving); Brief Visuospatial Memory Test-Revised (visual learning); Category fluency test, animal naming (speed of processing); Mayer-Salovey-Ca-ruso Emotional Intelligence Test, managing emotions branch (social cognition); and Continuous Performance Test, Identical Pairs version (attention/vigilance). The test battery is appropriate to measure cognitive change. Further tests are the Verbal Recognition Memory (VRM) test, the Rapid Visual information Processing (RVP) test, the Spatial Working Memory (SWM) test and the Digit Symbol Substitution Test (DSST).

Postpartum depression (PPD) is a complex mix of physical, emotional, and behavioural changes that happen in some women after giving birth. PPD is also known as major depressive disorder with peripartum onset. According to DSM-5 (Diagnostic and Statistical Manual of Mental Disorders, 5th Edition) criteria, PPD is diagnosed when major depressive disorder (MDD) symptoms begin during pregnancy or within four weeks of delivery. A patient/subject treated according to the present invention is preferably a woman diagnosed with PPD and >4 weeks postpartum. Further, the patient/subject will preferably be *9 months postpartum. Depressive aspects of PPD may be assessed by the HAM-D or the MADRS score. The Edinburgh Postnatal Depression Scale (EPDS) may also be used.

The Montgomery-Asberg Depression Rating Scale (MADRS) is a ten-item diagnostic questionnaire used to measure the severity of depressive episodes in patients/subjects with mood disorders (Montgomery, S. A., & Asberg, M. (1979). A new depression scale designed to be sensitive to change. The British Journal of Psychiatry 134, p. 382). It was designed as an adjunct to the Hamilton Rating Scale for Depression (HAM-D), which would be more sensitive to the changes brought on by antidepressants and other forms of treatment. Higher MADRS score indicates more severe depression. The items considered are apparent sadness; reported sadness; inner tension; reduced sleep; reduced appetite; concentration difficulties; lassitude; inability to feel; pessimistic thoughts; and suicidal thoughts, and each item yields a score of 0 to 6. The overall score ranges from 0 to 60.

A patient/subject may suffer from moderate or severe PPD as indicated by a Montgomery-Asberg Depression Rating Scale (MADRS) score of 20 or more or by a Hamilton Depression Rating Scale (HAM-D) score of 16 or more. It is further considered that the patient/subject may suffer from severe PPD as indicated by a Montgomery-Asberg Depression Rating Scale (MADRS) score of 35 or more or by a Hamilton Depression Rating Scale (HAM-D) score of 27 or more. The patient/subject may be diagnosed with a treatment-resistant form of PPD. A patient/subject treated according to the invention may have a Montgomery-Asberg Depression Rating Scale (MADRS) score of 20 or more or a 17-item Hamilton Depression Rating Scale (HAM-D) score of 16 or more.

Further, a patient/subject treated according to the invention may have a MADRS score of 28 or more or a HAM-D score of 22 or more. Still further, a patient/subject treated according to the invention may have a MADRS score of 35 or more or a HAM-D score of 25 or more. In addition to the above, PPD compromises maternal functioning. In particular the first year after childbirth marks a critical window for both mother and child. In most cases, mothers are the primary caregivers and are, therefore, responsible for the majority of the work related to infant care tasks.

Maternal functioning includes aspects of maternal competence relating to interactions with the infant(s) as well as maternal self-care. Maternal functioning, including the emotional aspect of mothering, is also important for the child's development. In fact, the quality of mother-child interaction in the year after birth affects infant development. High levels of maternal functioning are likely to correlate with positive infant development outcomes. Likewise, impaired functioning in the postpartum period might impede optimal infant development.

The Barkin Index of Maternal Functioning (BIMF) was designed to measure functioning in the year after childbirth. The BIMF is a 20-item self-report measure of functioning. Each item is assigned a score between 0 and 6 so that the maximum total score is 120. The higher the score, the better maternal functioning is rated. The BIMF identifies the key functional domains of a mother during the postnatal period as: self-care, infant care, mother-child interaction, psychological wellbeing of the mother, social support, management, and adjustment.

A BIMF score of 95 or below is considered herein as representing slightly compromised maternal functioning, score of 80 or below is considered herein as representing compromised maternal functioning, a score of 65 or below is considered herein as representing severely compromised maternal functioning. The invention in particular allows improving maternal functioning in patients/subjects having a score of 80 or below before treatment and in patients/subjects having a score of even 65 or below.

Symptoms such as anhedonia; emotional withdrawal and affective flattening are clustered together here as social/emotional withdrawal or detachment. Reduced social engagement is a further aspect associated with social/emotional withdrawal or detachment.

Anhedonia is the inability to experience pleasure. The patient/subject does not suffer from anhedonia if there is patient/subjectively no reduced ability to experience pleasure in usual activities. Anhedonia is mild in the case of slight reduction in pleasure from usually pleasurable activities; moderate in the case of significant reduction in pleasure from usually pleasurable activities or some pleasure from isolated activities retained; or severe in the case of complete inability to experience pleasure.

Anhedonia comprises consummatory (or liking) and anticipatory (or wanting) components. Consummatory pleasure refers to the “in the moment” pleasure experienced by the patient/subject directly engaged in an enjoyable activity, whereas anticipatory pleasure refers to the experience of pleasure related to future activities. Affective flattening characterises the patient/subjective sense of reduced intensity or range of feelings or emotions. The patient/subject does not show affective flattening if there is no sense of reduced intensity or range of feeling or emotions. It is mild in the case of slight constriction of range of affect, or transient reduction in range or intensity of feelings; moderate in the case of significant constriction of range or intensity of feelings with preservation of some emotions, e.g., inability to cry; and severe in the case of marked and pervasive constriction of range of affect or inability to experience usual emotions.

Emotional withdrawal or detachment is an inability or unwillingness to connect with other people on an emotional level. For example, the BPRS contains an item relating to emotional withdrawal, which is characterised as the deficiency in the patient/subject's ability to relate emotionally during the interview situation. According to the description of this BPRS item, there is no emotional withdrawal if there is no lack of emotional involvement shown by occasional failure to make reciprocal comments, occasionally appearing preoccupied, or smiling in a stilted manner, but spontaneously engages the interviewer most of the time.

There is a mild form of emotional withdrawal if there is a lack of emotional involvement shown by noticeable failure to make reciprocal comments, appearing preoccupied, or lacking in warmth, but responds to the interviewer when approached. It is moderate if the emotional contact is not present much of the interview because the patient/subject does not elaborate responses, fails to make eye contact, does not seem to care if the interviewer is listening, or may be preoccupied with psychotic material. It is moderately severe if, in addition, emotional contact is not present most of the interview. Severe forms are present if emotional participation is actively avoided by the patient/subject or if the patient/subject is frequently unresponsive or responds with yes/no answers or responds with only minimal affect. It is extremely severe if the patient/subject consistently avoids emotional participation, is unresponsive or responds with yes/no answers or may leave during the interview or just not respond at all.

Reduced social engagement characterises patient/subjective reports of reduced social and interpersonal engagement or interactions. There is no reduced social engagement if there are no reports of reduced social and interpersonal engagement or interactions. It is mild in the case of slight reduction in social engagement with no impairment in social or interpersonal function; moderate in the case of clear reduction in social engagement with some functional sequelae, e.g., avoiding some social engagements or conversations; and severe in the case of marked reduction in social interaction or avoidance of almost all forms of social contact, e.g., refusing to answer the phone or see friends or family.

Social/emotional withdrawal or detachment may be associated with one or more conditions or disorders, such as a mental disorder or a nervous system disorder or some other medical conditions. Mental or nervous system disorders which lead to, or are associated with, social/emotional withdrawal or detachment include disorders characterised by depressive episodes, for example Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorders, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); Posttraumatic Stress Disorder (PTSD); Pain Disorders, for example Chronic Pain and Fibromyalgia; Mental and Behavioural Disorders due to Psychoactive Substance Use, for example Substance Use Disorder (SUD); Psychotic Disorders, for example Schizophrenia; Dementia, for example Alzheimer's Dementia (AD); Dementia with Lewy Bodies (DLB); Vascular Dementia and Fronto-Temporal Dementia (FTD); Parkinson's Disease (PD); Eating Disorders; Autism Spectrum Disorder (ASD); Attention Deficit Hyperactivity Disorder (ADHD); and Personality Disorders, for example Schizotypal Personality Disorder and Borderline Personality Disorder (BPD).

The social/emotional withdrawal or detachment may also occur in a patient/subject suffering from sleep disturbance, for instance, insomnia. The social/emotional withdrawal or detachment may also occur in a patient/subject suffering from medical health conditions leading to an associated mental or nervous system condition including Traumatic Brain Injury (TBI).

Social/emotional withdrawal or detachment or individual aspects thereof, such as anhedonia, emotional withdrawal and affective flattening, may be evaluated by different instruments, such as questionnaires or scales. Questionnaires assess the mental status of a patient/subject based on observations made by the patient/subject himself/herself, caregivers or the clinician/physician administering the questionnaire. Questionnaires used to assess whether a patient/subject suffers from a particular mental or nervous system disorder may comprise items related to social/emotional withdrawal or detachment.

The Snaith-Hamilton Pleasure Scale (SHAPS) is a 14-item scale that measures anhedonia, i.e., the inability to experience pleasure. The items cover the domains of: social interaction, food and drink, sensory experience, and interest/pastimes. A score of 2 or less constitutes a “normal” score, while an “abnormal” score is defined as 3 or more. Each item has four possible responses: strongly disagree, disagree, agree, or strongly agree. Either of the “disagree” responses score one point, and either of the “agree” responses score 0 points. Thus, the final score ranges from 0 to 14. The SHAPS has adequate construct validity and satisfactory test-retest reliability. High internal consistency has also been reported. The SHAPS has been used for measuring anhedonia in depression, but it is also frequently used to assess anhedonia in other patient/subject groups.

In principle, the SHAPS measures hedonic tone during the last few days with 14 hypothetically formulated items. However, due to the hypothetical nature of the items an appropriate shorter recall period may also be applied for an earlier assessment time point. Alternatively or additionally, the Dimensional Anhedonia Rating Scale (DARS) measuring interest, motivation, effort and consummatory pleasure across four domains: hobbies, food/drink, social activities and sensory experience may be used for the assessment of anhedonia. It comprises 17 items assessing state anhedonia right now. The DARS is rated on a five-point Likert scale from 0 (not at all) to 4 (very much), higher values indicating less anhedonia. All items are summed up to a total score in the range of 0 to 68. For each of the four hedonic domains, hobbies (four items, sum score 0-16), food/drink (four items, sum score 0-16), social activities (four items, sum score 0-16) and sensory experiences (5 items, sum score 0-20), patients/subjects/patient/subjects are asked to provide two or three of their own favourite examples.

The Personality Inventory for DSM-5 (PID-5)—Adult is a 220 item self-rated personality trait assessment scale for adults age 18 and older. It assesses 25 personality trait facets including Anhedonia, Anxiousness, Attention Seeking, Callousness, Deceitfulness, Depressivity, Distractibility, Eccentricity, Emotional Lability, Grandiosity, Hostility, Impulsivity, Intimacy Avoidance, Irresponsibility, Manipulativeness, Perceptual Dysregulation, Perseveration, Restricted Affectivity, Rigid Perfectionism, Risk Taking, Separation Insecurity, Submissiveness, Suspiciousness, Unusual Beliefs and Experiences, and Withdrawal, with each trait facet consisting of 4 to 14 items.

The trait facet Anhedonia contains the items 1, 23, 26, 30R, 124, 155R, 157, 189 (reverse scored items are marked with the letter “R”), the trait facet Withdrawal contains the items 10, 20, 75, 82, 136, 146, 147, 161, 182, 186 and the trait facet Intimacy Avoidance contains the items 89, 97R, 108, 120, 145, 203. These three trait facets may be combined to yield the broader trait domain designated Detachment. The measure is completed by the individual prior to a visit with the clinician/physician. Each item asks the individual to rate how well the item describes him or her generally. Each item on the measure is rated on a 4-point scale. The response categories for the items are 0=very false or often false; 1=sometimes or somewhat false; 2=sometimes or somewhat true; 3=very true or often true. For items 7, 30, 35, 58, 87, 90, 96, 97, 98, 131, 142, 155, 164, 177, 210, and 215, the items are reverse-coded prior to entering into scale score computations.

The scores on the items within each trait facet should be summed and entered in the appropriate raw facet score box. In addition, the clinician/physician is asked to calculate and use average scores for each facet and domain. The average scores reduce the overall score as well as the scores for each domain to a 4-point scale, which allows the clinician/physician to think of the individual's personality dysfunction relative to observed norms. The average facet score is calculated by dividing the raw facet score by the number of items in the facet (e.g., if all the items within the “Anhedonia” facet are rated as being “sometimes or somewhat true,” then the average facet score would be 16/8=2, indicating moderate anhedonia). An average domain score is calculated by summing and then averaging the 3 facet scores contributing primarily to the specific domain. For example, if the average facet scores on Anhedonia, Intimacy Avoidance and Withdrawal (scales primarily indexing Detachment) are all 2, then the sum of these scores would be 6, and the average domain score would be 6/3=2. Higher average scores indicate greater dysfunction in a specific personality trait facet or domain. High scores on a facet or domain may indicate significant and problematic areas for the individual receiving care that might warrant further assessment, treatment, and follow up.

Key aspects observed with patients/subjects suffering from psychomotor retardation are reduced energy and activity and reduced motivation. Psychomotor retardation involves a slowing down of thought and a reduction of physical movements in an individual. Psychomotor impairment may cause a visible slowing of physical and emotional reactions. Psychomotor retardation may be associated with one or more conditions or disorders, such as a mental disorder or a nervous system disorder or some other medical conditions.

Mental or nervous system disorders which lead to, or are associated with, psychomotor retardation include disorders characterised by depressive episodes, for example, Major Depressive Disorder (MDD); Bipolar Disorder (BD), such as Bipolar I Disorder and Bipolar II Disorder; Postpartum Depression (PPD); Seasonal Affective Disorder and Persistent Depressive Disorder; Mental and Behavioural Disorders due to Psychoactive Substance Use, for example Substance Use Disorder (SUD); Psychotic Disorder, for example Schizophrenia; Dementia, for example Alzheimer's Dementia (AD); Dementia with Lewy Bodies (DLB); Vascular Dementia and Parkinson's Disease Dementia; Parkinson's Disease; Chronic Fatigue Syndrome. Psychomotor retardation may also occur in a patient/subject suffering from sleep disturbance, for instance, insomnia.

Psychomotor retardation may be assessed by measuring various aspects. These may include for instance various types of drawing tasks and tests, such as the trail making test (TMT), the digit symbol substitution test (DSST), or the Gibson Spiral Maze Test (GSM) and others which are known in the art. In the trail making test (TMT), for instance, patients/subjects must connect 25 circles that contain either numbers (TMT A) or a combination of numbers and letters (TMT B) in ascending order. Task requirements are similar for TMT-B, except that the patient/subject must alternate between numbers and letters (1, A, 2, B, 3, C and so on). The test thus evaluates processing speed (TMT A) or cognitive flexibility (TMT B). The score for each part represents the amount of time required to complete the task.

Another test that involves graphomotor ability is the Gibson Spiral Maze (GSM) assessing psychomotor speed only, and is not influenced by cognitive abilities. patients/subjects who complete the GSM must correctly trace through a spiral maze from a starting point to an endpoint without touching bordering lines. The digit symbol substitution test (DSST) also measures psychomotor speed and consists of digit-symbol pairs followed by a list of digits. Under each digit, the patient/subject should write down the corresponding symbol as fast as possible. The score consists in the number of symbols correctly reported in 90 s. A further example for a motor test is the finger tapping test.

Thus, certain tests combine measurements of both motor and cognitive aspects of psychomotor retardation, while further others assess only motor aspects. Analysis of speech may be a further indicator of psychomotor retardation. Major scales available to assess and measure include the severity of psychomotor retardation, the Salpetriere Retardation Rating Scale (SRRS) and the Motor Agitation and Retardation Scale (MARS). The Salpetriere Retardation Rating Scale (SRRS), developed by Widlocher assesses cognitive and motor aspects by fifteen items. The first three measure movement, specifically the quality of stride and slowness of limb, trunk, head, and neck movement. The next three items focus on speech including verbal flow, tone of voice, and length of response. Two items are designed to objectively measure cognitive function. These questions are based on the interview conversation and measure the patient's ability to approach and expand on topics. The further items are patient/subjective and assess rumination, fatigue, level of interest, perception of time, memory, and concentration. The last item of the scale relates to an overall assessment of the patient's psychomotor retardation. The items are scaled from 0 (symptom absence) to 4 (severe) based on the severity of the presenting symptom, for a total score range of 0 to 60.

The Motor Agitation and Retardation Scale (MARS) assesses motor aspects only. It was designed to assess psychomotor disturbances in depressive disorders. Psychomotor disturbances are divided into five major body categories including eyes, face, voice, limbs, and trunk with a total of 19 items on the scale. Items of the eyes category include direction of gaze, amount of blinking, staring, and eye movement. Items associated with the face category include facial expression and facial expressivity. The category of voice has items that include volume, slurring, tone and time for onset. Items under the limbs category include hand, foot, and leg movement, stride, motor slowness, and tension in hands. The trunk category items include posture, immobility, and axial movement. The severity of each item ranges from a 1 to a 4, with 4 being the most severe. Of the 19 items 9 relate to motor agitation and 10 items assess motor retardation. The retardation items include abnormal gait, immobility of trunk/proximal limbs, postural collapse, motor slowness (i.e. the limb and trunk category); lack of facial expressivity, downcast gaze (i.e. the eyes and face category); and reduced voice volume, slurring of speech, delayed speech onset, monotone speech (i.e. the voice category). The MARS scale offers a rapid clinical assessment of motor signs.

In an embodiment, the pharmaceutical composition/formulation of the invention is for use in the treatment of a patient with a AES-S score of at least 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or 72. In an embodiment, treatment of a patient with the pharmaceutical composition/formulation of the invention results in a decrease in AES-S score of at least 10, 20, 30, 40, 50, 60, 70, 80, 90 or 100%. In an embodiment, treatment of a patient with the pharmaceutical composition/formulation of the invention results in a decrease in AES-S score of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or 72 points. In an embodiment, the pharmaceutical composition/formulation of the invention is for use in the treatment of a patient with a MADRS score of at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60. In an embodiment, treatment of a patient with the pharmaceutical composition/formulation of the invention results in a decrease in MADRS score of at least 10, 20, 30, 40, 50, 60, 70, 80, 90 or 100%. In an embodiment, treatment of a patient with the pharmaceutical composition/formulation of the invention results in a decrease in MADRS score of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 points. In an embodiment, the pharmaceutical composition/formulation of the invention is for use in the treatment of a patient with a GAD-7 score of at least 10, 11, 12, 13, 14, 15, 16, 17, 18, 19, 20 or 21 points. In an embodiment, treatment of a patient with the pharmaceutical composition/formulation of the invention results in a decrease in GAD-7 score of at least 10, 20, 30, 40, 50, 60, 70, 80, 90 or 100%. In an embodiment, treatment of a patient with the pharmaceutical composition/formulation of the invention results in a decrease in GAD-7 score of at least 2, 3, 4, 5, 6, 7, 8, 9, 10, 11, 12, 13, 14, 15, 16, 17, 18, 19, 20 or 21 points. In an embodiment, the pharmaceutical composition/formulation of the invention is for use in the treatment of a patient with a PHQ-9 score of at least 10, 11, 12, 13, 14, 15, 16, 17, 18, 19, 20, 21, 22, 23, 24, 25, 26, or 27 points. In an embodiment, treatment of a patient with the pharmaceutical composition/formulation of the invention results in a decrease in PHQ-9 score of at least 10, 20, 30, 40, 50, 60, 70, 80, 90 or 100%. In an embodiment, treatment of a patient with the pharmaceutical composition/formulation of the invention results in a decrease in PHQ-9 score of at least 2, 3, 4, 5, 6, 7, 8, 9, 10, 11, 12, 13, 14, 15, 16, 17, 18, 19, 20, 21, 22, 23, 24, 25, 26, or 27 points.

### Pharmacokinetics

The applicant has funded double-blind, randomised, Phase 1, single ascending dose studies to evaluate the safety, tolerability and pharmacokinetic profile of intranasal 5-MeO-DMT hydrochloride and intranasal 5-MeO-DMT benzoate in healthy subjects. The results of these studies have surprisingly shown that only the benzoate salt of 5-MeO-DMT exhibits dose-proportional pharmacokinetics. It is desirable that a compound for use in treatment has dose-proportional pharmacokinetics, for example, to facilitate dose and dose regimen adjustment in patients. In an embodiment, there is provided a salt of 5-MeO-DMT with dose-proportional pharmacokinetics, optionally for use in the methods disclosed herein. In an embodiment, the salt of 5-MeO-DMT with dose-proportional pharmacokinetics is the benzoate or HBr salt, optionally the benzoate salt. A double-blind, randomised, Phase 1, single ascending dose study to evaluate the safety, tolerability and pharmacokinetic profile of a liquid intranasal 5-MeO-DMT HCl (5-MeO-DMT HCl, HPMC, water for injection (WFI) and a sodium hydroxide solution to adjust pH) pharmaceutical composition/formulation in healthy subjects was performed. The mean (+/−SD) 5-MeO-DMT plasma log concentration-time plot is shown in FIG. 1. It can be seen that 5-MeO-DMT HCl does not display dose-proportional pharmacokinetics, with the mean concentration profiles displayed for 5 mg, 8 mg, 10 mg, 11 mg and 14 mg all being substantially similar.

A double-blind, randomised, Phase 1, single ascending dose study to evaluate the safety, tolerability and pharmacokinetic profile of intranasal 5-MeO-DMT benzoate in healthy subjects was performed. The mean (+/−SD) 5-MeO-DMT plasma linear concentration-time plot and plasma log concentration-time plot are shown in FIGS. 2 and 3, respectively. The pharmacokinetics were shown to be approximately dose linear. No dose exceeded the maximum exposure limits defined by previous preclinical work in dogs: Cmax: 421 ng/mL or AUC 220 h·ng/mL. The mean (+/−SD) 5-MeO-DMT plasma linear concentration-time plot and plasma log concentration-time plot are shown in FIGS. 9 and 10, respectively. The mean Cmax was 29 ng/ml for the 12 mg dosage. The mean Tmax was 9.5 minutes whilst the mean half-life (T½) was 21 minutes. Bufotenin, the O-demethylated metabolite of 5-MeO-DMT, was only detected at very low levels at the 6 mg dose level after the 16 minutes time point.

It will be understood that references to ‘5-MeO-DMT’ herein mean 5-MeO-DMT free base, or a pharmaceutically acceptable salt, prodrug, hydrate, ester, co-crystal or deuterated form thereof, or a pharmaceutical composition/formulation comprising the aforementioned.

### DEFINITIONS

Terms such as “a”, “an,” and “the” are not intended to refer to only a singular entity but include the general class of which a specific example may be used for illustration.

As used herein, the term “about” refers to a value that is within 10% above or below the value being described.

As used herein, the terms “acute stress disorder” and “ASD” refer to a condition that arises as a response to a stressful event or situation of an exceptionally threatening or catastrophic nature, which is likely to cause pervasive distress in an individual (e.g., natural or man-made disaster, combat, serious accident, witnessing the violent death of others, or being the victim of torture, terrorism, rape, or other crime). Like PTSD, acute stress disorder is an anxiety disorder that involves a very specific reaction following exposure to a traumatic event or stressor. However, the duration of acute stress disorder is shorter than that for PTSD, such that the symptoms are present for at least one, two, or three days, but no more than four, five, or six weeks. For individuals exhibiting symptoms persisting for a longer period of time, a diagnosis of PTSD may be warranted.

The term “administration” or “administering” refers to a method of giving a dosage of a compound or pharmaceutical composition/formulation to a subject.

By “dysthymia” or “dysthymic disorder” is meant a chronically depressed mood that occurs for most of the day, more days than not, for at least two years. In children and adolescents, the mood may be irritable rather than depressed, and the required minimum duration is one year. During the two-year period (one year for children or adolescents), any symptom-free intervals last no longer than 2 months. During periods of depressed mood, at least two of the following additional symptoms are present: poor appetite or overeating, insomnia or hypersomnia, low energy or fatigue, low self-esteem, poor concentration, or difficulty making decisions, and feelings of hopelessness. The symptoms cause clinically significant distress or impairment in social, occupational (or academic), or other important areas of functioning. The diagnosis of dysthymia is not made if: the individual has ever had a manic episode, a mixed episode, a hypomanic episode; has ever met the criteria for a cyclothymic disorder; the depressive symptoms occur exclusively during the course of a chronic psychotic disorder (e.g., schizophrenia); or if the disturbance is due to the direct physiological effects of a substance or a general medical condition. After the initial two-years of dysthymic disorder, major depressive episodes may be superimposed on the dysthymic disorder (“double depression”). Diagnostic and Statistical Manual of Mental Disorders (OSM IV), American Psychiatric Press, 4th Edition, 1 994. Diagnostic guidance for psychological disorders can be found, for example, in the ICD-10 (The ICD-10 Classification of Mental and Behavioral Disorders: Diagnostic Criteria for Research, Geneva: World Health Organization, 1993) and the DSM-V (American Psychiatric Association. Diagnostic and Statistical Manual of Mental Disorders, Fifth Edition (DSM-V) Arlington, VA.; American Psychiatric Association, 2013).

As used herein, the term “psychological support” may refer to one or more of the following: therapy, psychotherapy, talk therapy, cognitive behavioral therapy (CBT), counseling, guided self-help and/or group therapy.

As used herein, the term “generalized anxiety disorder” refers to a condition characterized by excessive anxiety and worry (i.e., apprehensive expectation). Typically, the excessive anxiety and worry occur on more days than not for a period of time (e.g., one, two, three, or four months or more). The anxiety and worry can be associated with (i) restlessness, feeling keyed up, or on edge; and/or (ii) muscle tension.

The anxiety and worry can be associated with (a) a marked avoidance of situations in which a negative outcome could occur; (b) a marked time and effort preparing for situations in which a negative outcome could occur; (c) a marked procrastination in behavior or decision-making due to worries; and (d) repeatedly seeking reassurance due to worries. The anxiety, worry, or physical symptoms can cause clinically significant distress or impairment in social, occupational, or other important areas of functioning in many, but not necessarily all individuals with GAD.

As used herein, the terms “obsessive compulsive disorder,” “OCD,” and “anxiety and obsessive-compulsive spectrum disorders” refer to a condition characterized by obsessions and/or compulsions. Obsessions are recurrent and persistent thoughts, urges, or images that are experienced, at some time during the disturbance, as intrusive and unwanted and that usually cause marked anxiety or distress in which the obsessed individual attempts to ignore or suppress such thoughts, urges, or images, or to neutralize them with some other thought or action (i.e., by performing a compulsion). Compulsions are repetitive behaviors (e.g., hand washing, ordering, checking) or mental acts (e.g., praying, counting, repeating words silently) that the person feels driven to perform in response to an obsession, or according to rules that must be applied rigidly. The behaviors or mental acts are aimed at preventing or reducing anxiety or distress, or preventing some dreaded event or situation; however, these behaviors or mental acts either are not connected in a realistic way with what they are designed to neutralize or prevent, or are clearly excessive. Typically the obsessions or compulsions are time consuming (for example, take more than 1 hour a day), or cause clinically significant distress or impairment in social, occupational, or other important areas of functioning.

As used herein, the term “panic disorder” refers to a condition characterized by recurrent and unexpected panic attacks. Panic disorder includes both panic disorder with agoraphobia and panic disorder without agoraphobia. Subjects with this condition can exhibit one or both of the following: (i) a persistent concern or worry about additional panic attacks or their consequences (e.g., losing control, having a heart attack, going crazy); and/or (ii) significant maladaptive change in behavior related to the attacks (e.g., behaviors designed to avoid having panic attacks), which may include agoraphobic avoidance.

As used herein, the terms “pharmacologically effective amount,” “therapeutically effective amount,” and the like, when used in reference to a therapeutic formulation, refer to a quantity sufficient to, when administered to the subject, including a mammal, for example a human, effect beneficial or desired results, such as clinical results. For example, in the context of treating depression, described herein, these terms refer to an amount of the pharmaceutical composition/formulation sufficient to achieve a treatment response as compared to the response obtained without administration of the formulation. The quantity of a given pharmaceutical composition/formulation described herein that will correspond to such an amount may vary depending upon various factors, such as the given agent, the formulation, the route of administration, the type of disease or disorder, the identity of the subject (e.g., age, sex, weight) or host being treated, and the like. An “effective amount,” “pharmacologically effective amount,” or the like, of a pharmaceutical composition/formulation of the present disclosure, also include an amount that results in a beneficial or desired result in a subject as compared to a control (e.g., a decrease in the score on the Montgomery-Asberg Depression Rating Scale).

As used herein, the terms “post traumatic stress disorder” and “PTSD” refer to a condition that arises as a delayed and/or protracted response to a stressful event or situation (either short- or long-lasting) of an exceptionally threatening or catastrophic nature, which is likely to cause pervasive distress in an individual (e.g., natural or man-made disaster, combat, serious accident, witnessing the violent death of others, or being the victim of torture, terrorism, rape, or other crime). Predisposing factors such as personality traits (e.g., compulsive, asthenic) or previous history of neurotic illness may lower the threshold for the development of the condition or aggravate its course, but they are neither necessary nor sufficient to explain its occurrence. PTSD is a less frequent and more enduring consequence of psychological trauma than the more frequently seen acute stress response. PTSD has been recognized in the past as railway spine, stress syndrome, shell shock, battle fatigue, traumatic war neurosis, and post-traumatic stress syndrome. Diagnostic symptoms include re-experiencing original trauma(s), by means of flashbacks or nightmares; avoidance of stimuli associated with the trauma; and increased arousal, such as difficulty falling or staying asleep, anger, and hypervigilance. Formal diagnostic criteria (DSM-V, DSM-IV, and/or ICD-9) require that the symptoms last more than one month and cause significant impairment in social, occupational, or other important areas of functioning (e.g., problems with work and/or relationships). Formal diagnostic criteria can include: (i) intrusion symptoms that are associated with the traumatic event (e.g., (a) spontaneous or cued recurrent, involuntary, and intrusive distressing memories of the traumatic event; (b) recurrent distressing dreams in which the content and/or affect of the dream is related to the event; (c) dissociative reactions (e.g., flashbacks) in which the individual feels or acts as if the traumatic event were recurring (such reactions may occur on a continuum, with the most extreme expression being a complete loss of awareness of present surroundings; (d) intense or prolonged psychological distress at exposure to internal or external cues that symbolize or resemble an embodiment of the traumatic event; and/or (e) marked physiological reactions to reminders of the traumatic event); (ii) persistent avoidance of stimuli associated with the traumatic event (e.g., (a) thoughts, feelings, or physical sensations that arouse recollections of the traumatic event; (b) activities, places, physical reminders, or times (e.g., anniversary reactions) that arouse recollections of the traumatic event; and/or (c) people, conversations, or interpersonal situations that arouse recollections of the traumatic event); (iii) negative alterations in cognitions and mood that are associated with the traumatic event (e.g., (a) inability to remember an important embodiment of the traumatic event (typically dissociative amnesia); (b) persistent and exaggerated negative expectations about one's self, others, or the world; (c) persistent distorted blame of self or others about the cause or consequences of the traumatic event; (d) pervasive negative emotional state (e.g., fear, horror, anger, guilt, or shame); (e) markedly diminished interest or participation in significant activities; (f) feeling of detachment or estrangement from others; and/or (g) persistent inability to experience positive emotions (e.g., unable to have loving feelings, psychic numbing); and (iv) alterations in arousal (i.e., hyperarousal) and reactivity that are associated with the traumatic event (e.g., (a) irritable, angry, or aggressive behavior; (b) reckless or self-destructive behavior; (c) hypervigilance; (d) exaggerated startle response; (e) problems with concentration; and/or (f) sleep disturbance (e.g., difficulty falling or staying asleep, or restless sleep)). Formal diagnostic criteria can further include that the duration of disturbance is more than a certain period of time (e.g., one month, three months, or six months) and that the disturbance causes clinically significant distress or impairment in social, occupational, or other important areas of functioning. In a small proportion of patients the condition may show a chronic course over many years and a transition to an enduring personality change. The three main symptoms associated with PTSD are (1) “reliving” the traumatic event, such as flashbacks, nightmares, intrusive thoughts and recollections, (2) avoidance behaviors and emotional numbing, and (3) hypersensitivity such as an inability to sleep, anxious feelings, overactive startle response, hyperarousal, hypervigilance, irritability, and outbursts of anger.

As used herein, the terms “psychological disorder” and “psychological condition” refer to a condition characterized by a disturbance in one's emotional or behavioral regulation that reflects a dysfunction in the psychological, biological, or developmental processes underlying mental function. Psychological disorders include, but are not limited to depressive disorders (major depression, treatment resistant depression, melancholic depression, atypical depression, or dysthymia), anxiety disorders (end of life anxiety, generalized anxiety disorder, panic disorder, social anxiety, post-traumatic stress disorder, acute stress disorder, obsessive compulsive disorder, or social phobia), addictions (e.g., substance abuse, e.g., alcoholism, tobacco abuse, or drug abuse)), eating disorders (e.g., anorexia nervosa, bulimia nervosa, and binge eating disorder) and compulsive behavior disorders (e.g., primary impulse-control disorders or obsessive-compulsive disorder). Psychological disorders can be any psychological condition associated with one or more symptoms, e.g., somatic symptoms (e.g., chronic pain, anxiety disproportionate to severity of physical complaints, pain disorder, body dysmorphia, conversion (i.e., loss of bodily function due to anxiety), hysteria, or neurological conditions without identifiable cause), or psychosomatic symptoms (e.g., back pain, fibromyalgia, migraines, and chronic fatigue syndrome). Psychological disorders also include repetitive body-focused behaviors, such as tic disorders (e.g., Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder). In some cases, development of a psychological disorder is associated with or characterized by a prodromal symptom, such as depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses.

As used herein, the terms “social phobia” and “social anxiety disorder” refer to a condition characterized by fear or anxiety associated with one or more social situations. Subjects with this condition typically exhibit a marked fear or anxiety about one or more social situations in which the person is exposed to possible scrutiny by others. Examples include social interactions (e.g., having a conversation), being observed (e.g., eating or drinking), or performance in front of others (e.g., giving a speech). Typically, an individual with this condition (i) fears that he or she will act in a way, or show anxiety symptoms that will be negatively evaluated (i.e., be humiliating, embarrassing, lead to rejection, or offend others); (ii) the social situations almost invariably provoke immediate fear or anxiety; (iii) the social situations are avoided or endured with intense fear or anxiety; and (iv) the fear or anxiety is out of proportion to the danger posed by the social situation. In children, the fear or anxiety may be expressed by crying, tantrums, freezing, clinging, shrinking or refusal to speak in social situations. The fear, anxiety, and avoidance can cause clinically significant distress or impairment in social, occupational, or other important areas of functioning.

As used herein, the terms “treat,” “treating,” or “treatment” refer to administration of a compound or pharmaceutical composition/formulation for a therapeutic purpose. To “treat a disorder” or use for “therapeutic treatment” refers to administering treatment to a patient already suffering from a disease to ameliorate the disease or one or more symptoms thereof to improve the patient's condition (e.g., by reducing one or more symptoms of inflammation). The term “therapeutic” includes the effect of mitigating deleterious clinical effects of certain inflammatory processes (i.e., consequences of the inflammation, rather than the symptoms of inflammation). The methods of the invention can be used as a primary prevention measure, i.e., to prevent a condition or to reduce the risk of developing a condition. Prevention refers to prophylactic treatment of a patient who may not have fully developed a condition or disorder, but who is susceptible to, or otherwise at risk of, the condition. Thus, in the claims and embodiments, the methods of the invention can be used either for therapeutic or prophylactic purposes.

By “major depressive disorder” is meant a clinical course that is characterized by one or more major depressive episodes in an individual without a history of manic, mixed, or hypomanic episodes. The diagnosis of unipolar depression is not made if: manic, mixed, or hypomanic episodes develop during the course of depression; if the depression is due to the direct physiological effects of a substance; if the depression is due to the direct physiological effects of a general medical condition; if the depression is due to a bereavement or other significant loss (“reactive depression”); or if the episodes are better accounted for by schizoaffective disorder and are not superimposed on schizophrenia, schizophreniform disorder, delusional disorder, or psychotic disorder. If manic, mixed, or hypomanic episodes develop, then the diagnosis is changed to a bipolar disorder. Depression may be associated with chronic general medical conditions (e.g., diabetes, myocardial infarction, carcinoma, and stroke). Generally, unipolar depression is more severe than dysthymia. The essential feature of a major depressive episode is a period of at least two to 15 weeks during which there is either depressed mood or loss of interest or pleasure in nearly all activities. In children and adolescents, the mood may be irritable rather than sad. The episode may be a single episode or may be recurrent. The individual also experiences at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation, plans, or attempts. Each symptom must be newly present or must have clearly worsened compared with the person's pre-episode status. The symptoms must persist for most of the day, nearly every day, for at least two consecutive weeks, and the episode must be accompanied by clinically significant distress or impairment in social, occupational (or academic), or other important areas of functioning (Diagnostic and Statistical Manual of Mental Disorders (OSM IV), American Psychiatric Press, 4th Edition, 1994). Diagnostic guidance for psychological disorders can be found, for example, in the ICD-10 (The ICD-10 Classification of Mental and Behavioral Disorders: Diagnostic Criteria for Research, Geneva: World Health Organization, 1993) and the DSM-V (American Psychiatric Association. Diagnostic and Statistical Manual of Mental Disorders, Fifth Edition (DSM-V) Arlington, VA.; American Psychiatric Association, 2013).

As used herein, the term “free flowing” refers to the ability of the plurality of solid particles to move in unbroken continuity, similar to a fluid (e.g., the individual solid particles within a plurality of solid units do not significantly adhere or stick to one another), to permit insufflation into a nasal cavity.

As used herein, “stable” refers to the ability of the therapeutic agent (e.g., a 5-MeO-DMT) to maintain its therapeutic efficacy (e.g., all or the majority of its intended biological activity and/or physiochemical integrity) over extended periods of time. Stable pharmaceutical compositions or formulations exhibit physical integrity and biological activity and a reduced susceptibility to chemical transformation (e.g., oxidation) prior to administration into a patient. Stable drug pharmaceutical compositions or formulations have a shelf life at about 5 °C and/or at about 25° C. of equal to or greater than 3, 6, 12, 18, or 24 months.

As used herein, “state-stable” refers to the ability of the therapeutic agent (e.g., amorphous 5-MeO-DMT) to substantially maintain its amorphous state over an extended period of time. For example, a state-stable amorphous solid maintains at least 75%, 85%, 90%, or 95% (w/w) of its amorphous form (i.e., resisting crystallisation) under storage conditions of between 2 °C and 25 °C and a relative humidity of 60% RH or below for a period of 1, 3, 6, or 12 months

The term “extended-release” refers to a drug pharmaceutical composition/formulation that provides for gradual release of a drug over an extended period of time, e.g., 2-20 minutes or more, compared to an immediate release pharmaceutical composition/formulation of the same drug, such that the active agent (e.g., a 5-MeO-DMT, or a pharmaceutically acceptable salt thereof) formulated in a unit dosage form has a dissolution release profile in which at least 10-80% (e.g., 10-60%, 10-40%, 10-20%, 20-80%, 40-80%, or 60-80%) of the agent is released within the 20 minutes of testing. Preferably, although not necessarily, extended release results in substantially constant blood levels of a drug over an extended time period that are within the therapeutic range for the disease being treated. Preferably an extended release pharmaceutical composition/formulation of a 5-MeO-DMT yields plasma 5-MeO-DMT levels that fall within a concentration range that is between, for example, 5-45 ng/ml, 5-40 ng/ml, 5-35 ng/ml, 5-30 ng/ml, 5-25 ng/ml, 5-20 ng/ml, 10-50 ng/ml, 15-50 ng/ml, 20-50 ng/ml, 25-50 ng/ml, 30-50 ng/ml, 35-50 ng/ml, 40-50 ng/ml, 10-40 ng/ml, or 10-30 ng/ml.

By “immediate release” is meant a mode of releasing the active agent (e.g., a 5-MeO-DMT, or a pharmaceutically acceptable salt thereof) formulated in a unit dosage form that has a dissolution release profile in which at least 80%, 85%, 90%, 95%, or 99% of the agent is released within the first two minutes of testing.

As used herein, the term “residence time” refers to a time period during which a compound, such 5-MeO-DMT, is present in nasal cavity, for example along the nasal cilia and mucus layer The residence time of the compound (e.g., 5-MeO-DMT) may be formulated to have an extended residence time of at least 10 minutes, 15 minutes, 20 minutes, 25 minutes, 30 minutes or greater compared to an immediate release pharmaceutical composition/formulation which has a residence time of fewer than 10 minutes, 8 minutes, 6 minutes, 5 minutes, or 2 minutes.

As used herein, the term “treating” refers to administering the dry powder pharmaceutical composition/formulation for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease (e.g., depression and/or alcohol use disorder) to ameliorate the disease and improve the patient's condition. The term “treating” also includes treating a patient to delay progression of a disease or its symptoms. Thus, in the claims and embodiments, treating is the administration to a patient either for therapeutic or prophylactic purposes.

As used herein, the term “amount sufficient to” refers to a quantity of the dry pharmaceutical composition/formulation sufficient to, when administered to the subject, including a mammal, for example a human, effect beneficial or desired results, such as clinical results. For example, in the context of treating depression, described herein, these terms refer to an amount of the pharmaceutical composition/formulation sufficient to achieve a treatment response as compared to the response obtained without administration of the formulation. The quantity of a given pharmaceutical composition/formulation described herein that will correspond to such an amount may vary depending upon various factors, such as the given agent, the formulation, the route of administration, the type of disease or disorder, the identity of the subject (e.g., age, sex, weight) or host being treated, and the like. An “amount sufficient to,” or the like, of a pharmaceutical composition/formulation of the present disclosure, also includes an amount that results in a beneficial or desired result in a subject as compared to a control (e.g., a decrease in the score on the Montgomery-Asberg Depression Rating Scale).

### BRIEF DESCRIPTION OF THE DRAWINGS

FIG. 1 shows the mean (+/−SD) 5-MeO-DMT HCl plasma log concentration-time plot.

FIG. 2 shows the mean (+/−SD) 5-MeO-DMT benzoate plasma linear concentration-time plot.

FIG. 3 shows the mean (+/−SD) 5-MeO-DMT benzoate plasma log concentration-time plot.

FIG. 4 shows an XRPD for the spray dried dispersion (SDD) of Example 1.

FIG. 5 shows a DSC thermogram for the SDD of Example 1.

FIG. 6 shows an XRPD for the SDD of Example 2, pre and post dynamic vapour sorption (DVS).

FIG. 7 shows a DVS isotherm for the SDD of Example 2.

FIG. 8 shows a DSC thermogram for the SDD of Example 2.

FIG. 9 shows an XRPD for the SDD of Example 3.

FIG. 10 shows a DSC thermogram for the SDD of Example 3.

FIG. 11 shows an XRPD for the SDD of Example 4.

FIG. 12 shows a DSC thermogram for the SDD of Example 4.

FIG. 13 shows an XRPD for the SDD of Example 5.

FIG. 14 shows an XRPD for the SDD of Example 6.

FIG. 15 shows the dissolution profile of the SDD of Example 6.

FIG. 16 shows an XRPD for the SDD of Example 7.

FIG. 17 shows an XRPD for the SDD of Example 8.

FIG. 18 shows an XRPD for the SDD of Example 9.

FIG. 19 shows an XRPD for the SDD of Example 10.

FIG. 20 shows a DSC thermogram for the SDD of Example 10.

FIG. 21 shows the dissolution profile for the SDD of Example 10.

FIG. 22 shows an XRPD for the SDD of Example 11.

FIG. 23 shows a DSC thermogram for the SDD of Example 11.

FIG. 24 shows the dissolution profile for the SDD of Example 11.

FIG. 25 shows an XRPD for the SDD of Example 12.

FIG. 26 shows the dissolution profile for the SDD of Example 12.

FIG. 27 shows a DSC thermogram for the SDD of Example 12.

FIG. 28 shows an XRPD for the SDD of Example 13.

FIG. 29 shows a DSC thermogram for the SDD of Example 13.

FIG. 30 shows the dissolution profile for the SDD of Example 13.

FIG. 31 shows an XRPD of the SDD of Example 14.

FIG. 32 shows a DSC thermogram for the SDD of Example 14.

FIG. 33 shows the dissolution profile for the SDD of Example 14.

FIG. 34 shows an XRPD of the SDD of Example 15.

FIG. 35 shows a DSC thermogram for the SDD of Example 15.

FIG. 36 shows the dissolution profile for the SDD of Example 15.

FIG. 37 shows an XRPD of the lyophilized dispersion of Example 18.

FIG. 38 shows a DSC thermogram for the lyophilized dispersion of Example 18.

FIG. 39 shows an XRPD of the lyophilized dispersion of Example 19.

FIG. 40 shows a DSC thermogram for the lyophilized dispersion of Example 19.

FIG. 41 shows an XRPD of the lyophilized dispersion of Example 20.

FIG. 42 shows a DSC thermogram for the lyophilized dispersion of Example 20.

FIG. 43 shows an XRPD of the lyophilized dispersion of Example 21.

FIG. 44 shows a DSC thermogram for the lyophilized dispersion of Example 21.

FIG. 45 shows an XRPD of the lyophilized dispersion of Example 22.

FIG. 46 shows a DSC thermogram for the lyophilized dispersion of Example 22.

FIG. 47 shows an XRPD of the lyophilized dispersion of Example 23.

FIG. 48 shows a DSC thermogram for the lyophilized dispersion of Example 23.

FIG. 49 shows an XRPD diffractogram for the HBr pharmaceutical composition/formulation of Example 16 at T=0, T=1 month post storage at 25 °C/60% RH and T=1 month post storage at 2-8 °C.

FIG. 50 shows an XRPD diffractogram for the HCl pharmaceutical composition/formulation of Example 17 at T=0, T=1 month post storage at 25 °C/60% RH and T=1 month post storage at 2-8 °C.

FIG. 51 shows an XRPD diffractogram for the HBr pharmaceutical composition/formulation of Example 16 at T=0, T=2 months post storage at 25 °C/60% RH and T=2 months post storage at 2-8 °C.

FIG. 52 shows an XRPD diffractogram for the HCl pharmaceutical composition/formulation of Example 17 at T=0, T=2 months post storage at 25 °C/60% RH and T=2 months post storage at 2-8 °C.

FIG. 53 shows the particle size distribution of a 5-MeO-DMT SDD as Bulk Material (Red) and ExDevice (Green).

FIG. 54 shows the nasal deposition profile for a 5-MeO-DMT SDD delivered via an active delivery nasal delivery device.

FIG. 55 shows the nasal deposition profile for a 5-MeO-DMT SDD delivered via a passive delivery nasal delivery device.

FIG. 56 shows the dissolution profile of a 5-MeO-DMT pharmaceutical composition/formulation comprising HPMC, isomalt and methyl cellulose.

FIG. 57 shows the dissolution profile of a 5-MeO-DMT pharmaceutical composition/formulation comprising sorbitol and methyl cellulose.

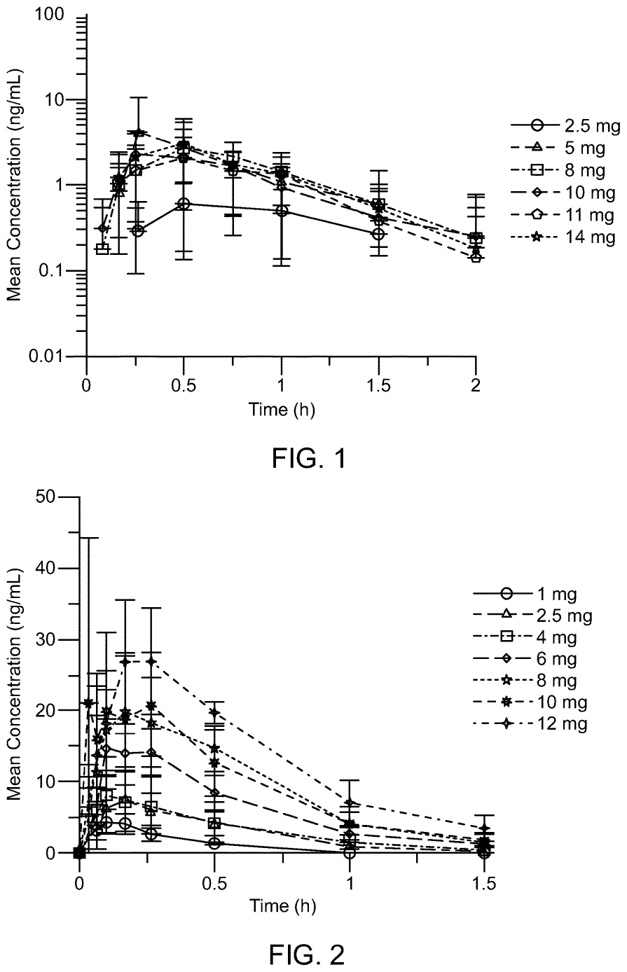

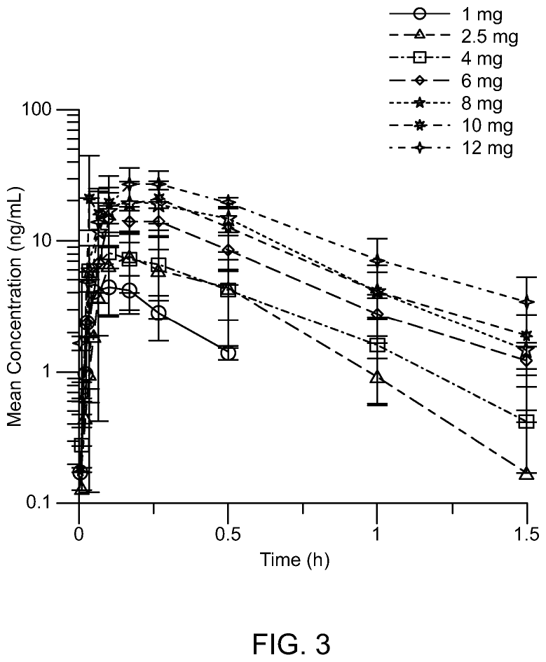

### EXAMPLES

### Example 1: Spray Drying of 5-MeO-DMT Hydrobromide Salt with HPMC

Spray drying 5-MeO-DMT hydrobromide and HPMC (Pharmacoat 606) in water produced a 50% wt:wt API to excipient spray dried dispersion (SDD). The spray drying parameters were as below:

Feed Solution

| | |
|---|---|
| Sample Reference | 30130-01-01 |
| HPMC (as Pharmacoat 606) | 1.01 g |
| 5-MeO-DMT hydrobromide | 1.05 g |
| Water (deionized) | 40.12 g |

Spray Drying Parameters

| | |
|---|---|
| Instrument | Procept |
| Nozzle | 2 fluid nozzle |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Gas Pressure (bar) | 1.2 |

Yield

| | |
|---|---|
| Mass of SDD Produced (g) | 1.54 |
| Yield (%) | 75 |

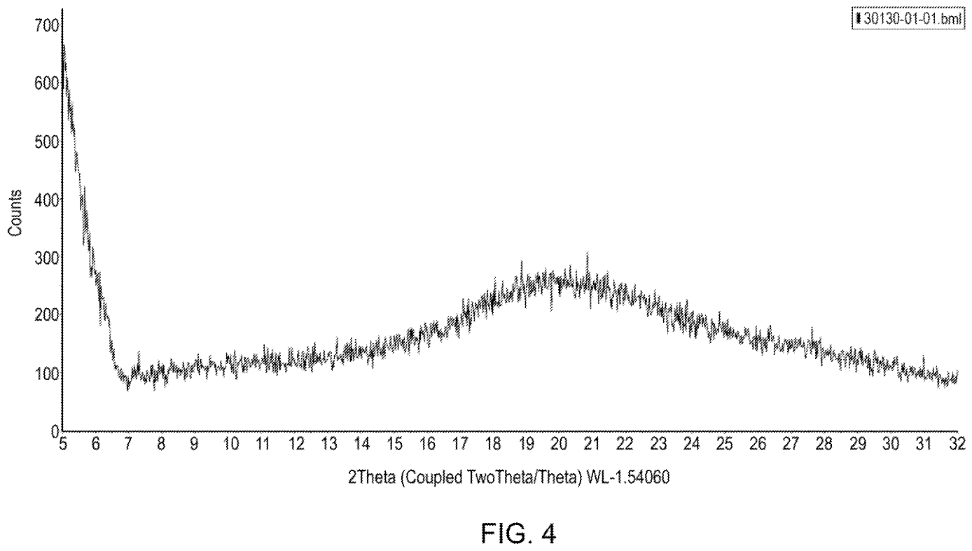

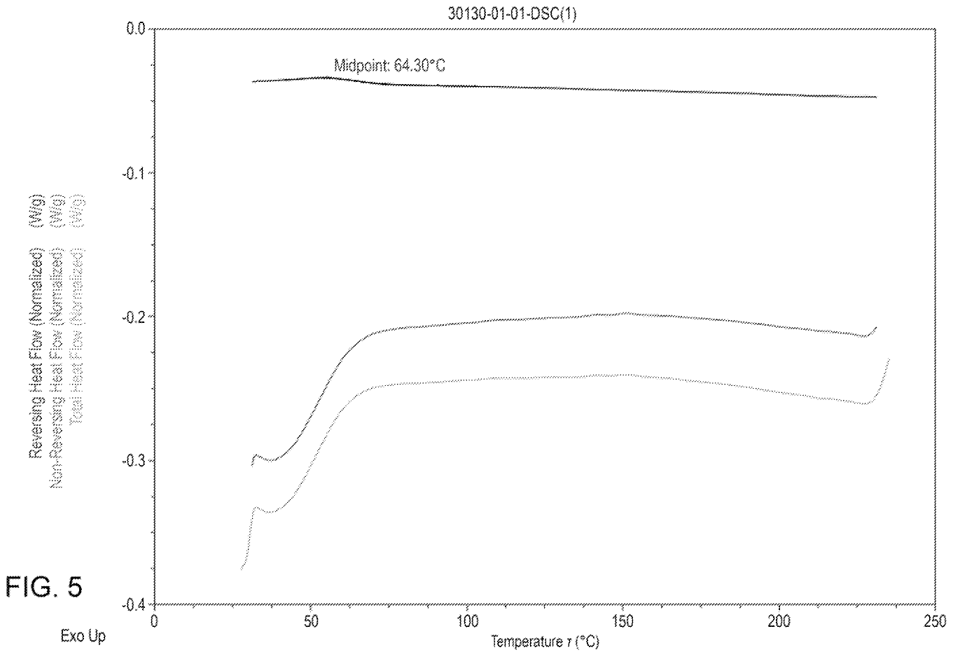

The SDD produced was amorphous, as shown by X-ray powder diffractogram (XRPD) analysis (FIG. 4), and the absence of an enthalpy of melting when the SDD was examined by differential scanning calorimetry (DSC) (FIG. 5).

### Example 2: Spray Drying of 5-MeO-DMT Oxalate Salt with HPMC

Spray drying 5-MeO-DMT oxalate and HPMC (Pharmacoat 606) in water produced a 50% wt:wt API to excipient SDD. The spray drying parameters were as below:

Feed Solution

| | |
|---|---|
| Sample Reference | 31030-02-01 |
| HPMC (as Pharmacoat 606) | 1.02 g |
| 5-MeO-DMT Oxalate | 1.01 g |
| Water (deionized) | 40.01 g |

Spray Drying Parameters

| | |
|---|---|
| Instrument | Procept |
| Nozzle | 2 fluid nozzle |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Gas Pressure (bar) | 1.2 |

Yield

| | |
|---|---|
| Mass of SDD Produced (g) | 1.32 |
| Yield (%) | 65 |

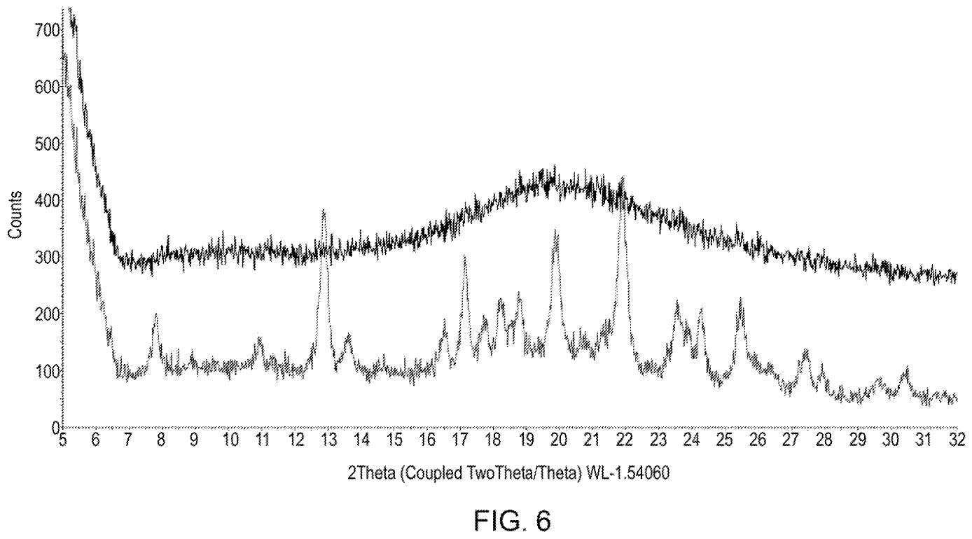

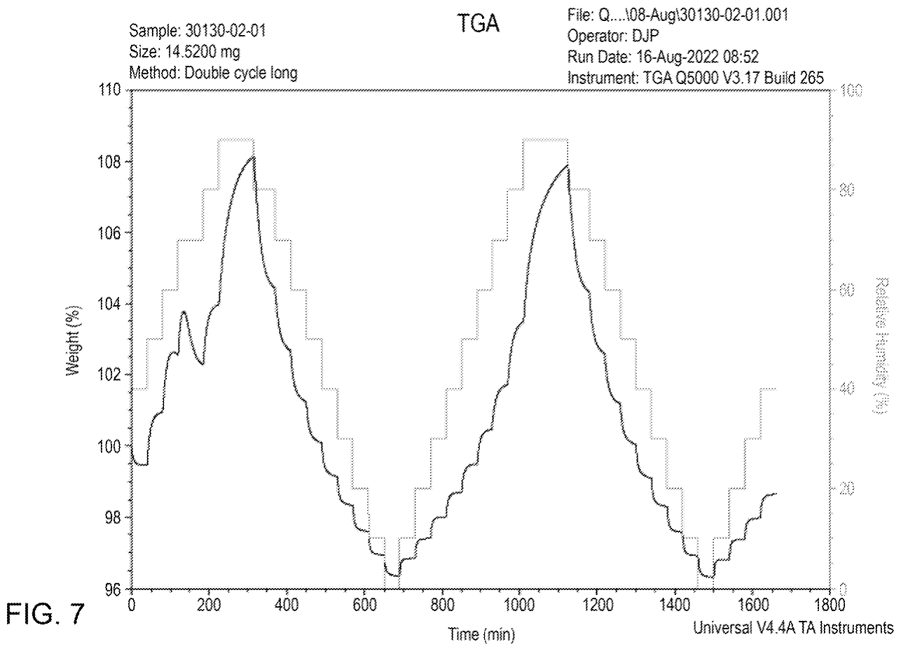

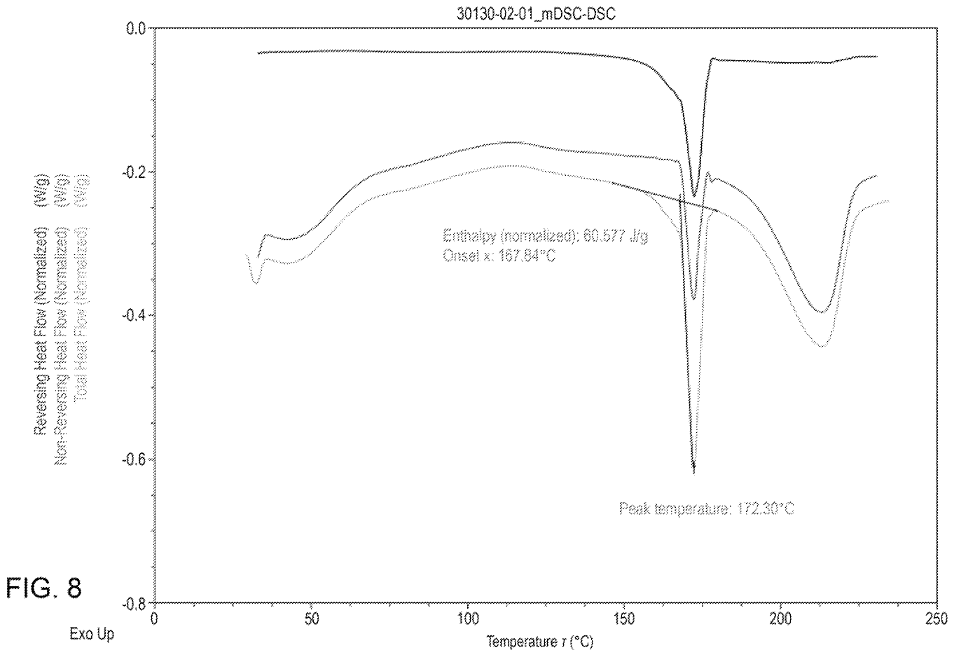

The SDD produced was a physically state-unstable amorphous SDD that underwent recrystallization at a relative humidity of above ~60%, as shown by dynamic vapour sorption (DVS) analysis. The XRPD analysis of the SDD pre and post DVS are shown in FIG. 6, the SDD is amorphous pre DVS and partially crystalline post DVS. The DVS isotherm is shown in FIG. 7 and the DSC thermogram is shown in FIG. 8.

### Example 3: Spray Drying of 5-MeO-DMT Hydrobromide Salt with PVP

Spray drying 5-MeO-DMT hydrobromide and polyvinylpyrrolidone (PVP) in water produced a 50% wt:wt API to excipient SDD. The spray drying parameters were as below:

Feed Solution

| | |
|---|---|
| Sample Reference | 31030-05-01 |
| PVP K30 | 0.253 g |
| 5-MeO-DMT Hydrobromide | 0.250 g |
| Water (deionized) | 10.221 g |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |

Yield

| | |
|---|---|
| Mass of SDD Produced (g) | 0.36 |
| Yield (%) | 65 |

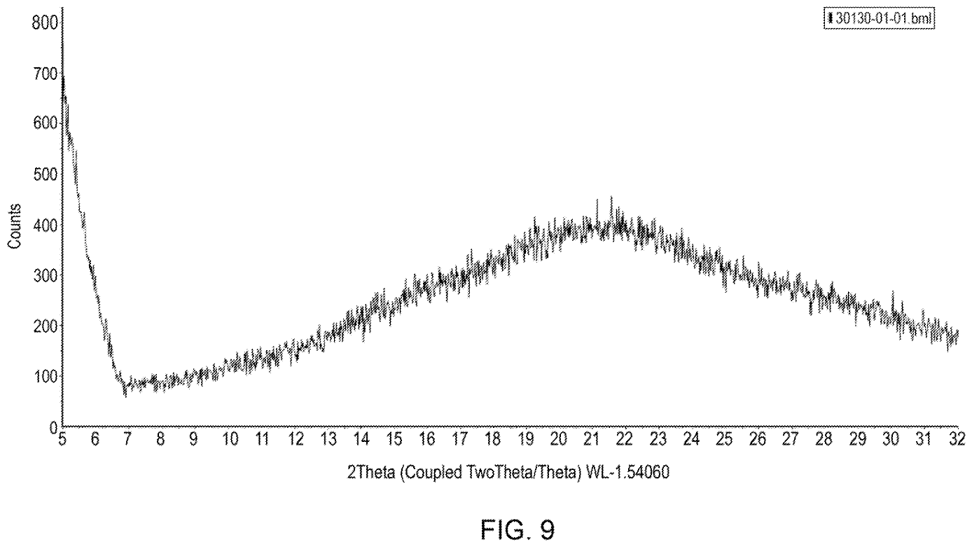

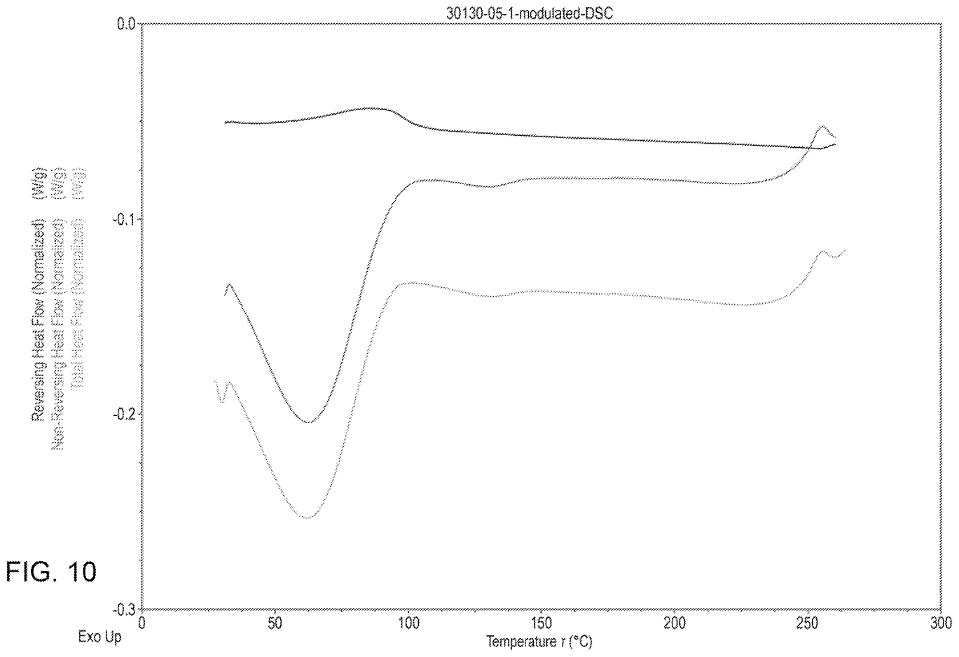

The SDD produced was amorphous, as shown by XRPD analysis (FIG. 9), and the absence of an enthalpy of melting when the SDD was examined by DSC (FIG. 10).

### Example 4: Spray Drying of 5-MeO-DMT Benzoate Salt with Trehalose

Spray drying 5-MeO-DMT benzoate and trehalose in water produced a 50% wt:wt API to excipient SDD. The spray drying parameters were as below:

Feed Solution

| | |
|---|---|
| Sample Reference | 31030-07-01 |
| Trehalose | 0.253 g |
| 5MeO DMT Benzoate | 0.249 g |
| Water (deionized) | 10.001 g |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |

Yield

| | |
|---|---|
| Mass of SDD Produced (g) | 0.222 |
| Yield (%) | 44 |

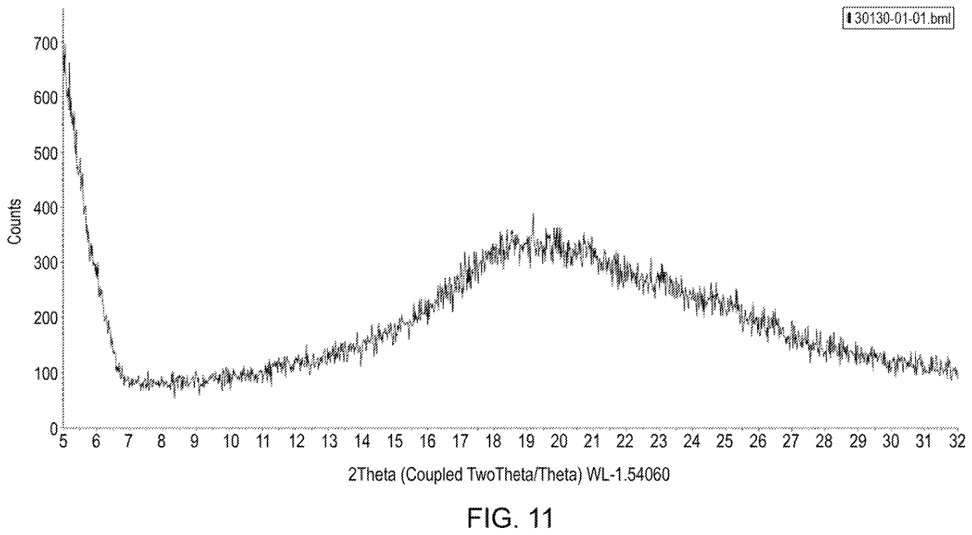

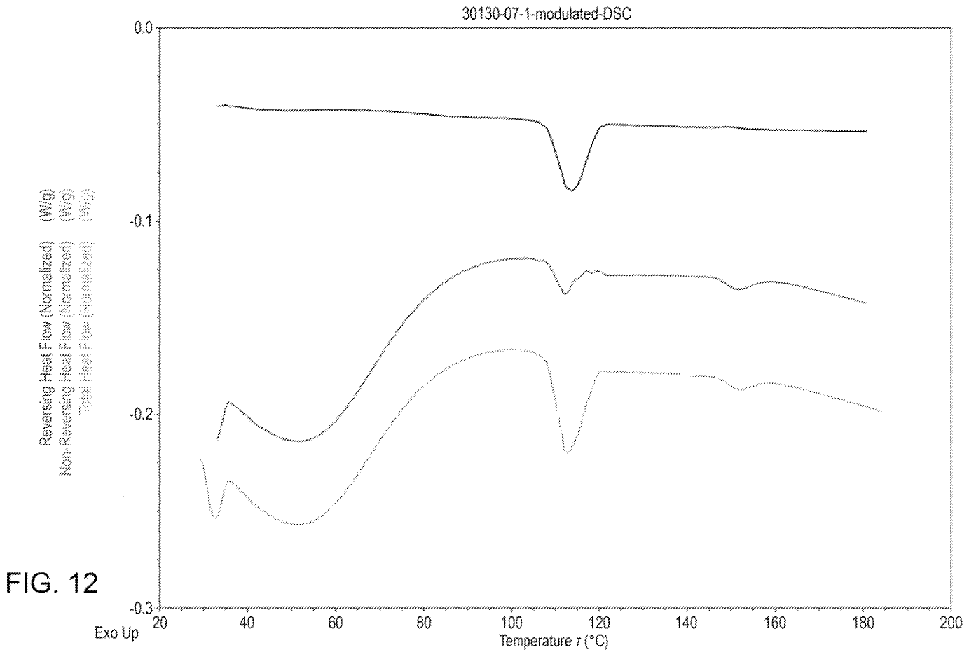

The SDD produced was predominantly amorphous by XRPD (FIG. 11), however, there was evidence of partially crystalline material in the XRPD and an enthalpy of melting observed in the DSC (FIG. 12) indicating possible physical instability of the SDD.

### Example 5: Spray Drying of 5-MeO-DMT Benzoate Salt with HPMC Mixtures

Spray drying of 5-MeO-DMT benzoate with a mixture of HPMC 2910 in water produced a 50% wt:wt API to excipient SDD. The spray drying parameters were as below:

Feed Solution

| | |
|---|---|
| Sample Reference | 30130-08-01 |
| Pharmacoat 606 | 1.19 g |
| Metolose 60SH50 | 1.19 g |
| 5-MeO-DMT Benzoate | 2.38 g |
| Water (deionized) | 95.24 g |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |

Yield

| | |
|---|---|
| Yield (%) | 34.7 |

The process for producing the feed solution was as follows: the required mass of water was weighed into a 50 mL vial. The required mass of HPMC (Pharmacoat 606) and metolose were added to the water whilst stirring and allowed to fully dissolve. Once dissolved the required mass of API was transferred into the solution and allowed to dissolve. Once dissolved the feed solution was spray dried immediately.

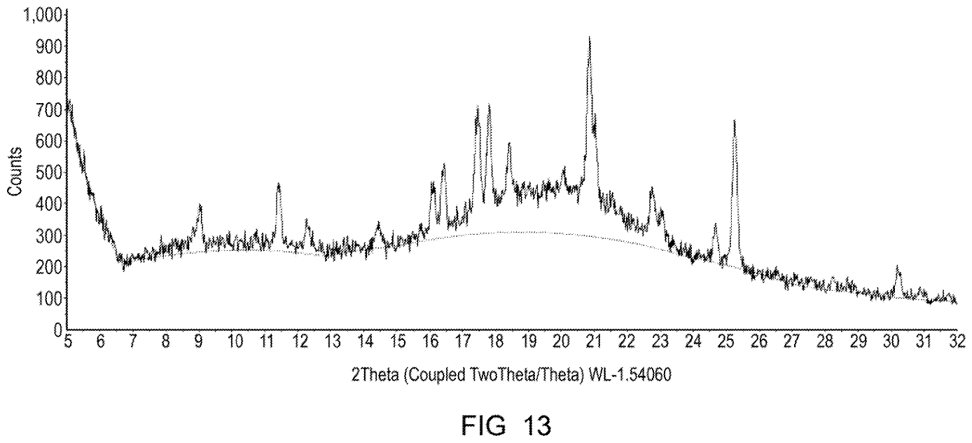

The SDD produced was partially crystalline (FIG. 13) and furthermore, at 25% loading of metolose, the yield was quite low.

### Example 6: Spray Drying of 5-MeO-DMT Benzoate Salt with HPMC Mixtures

Spray drying of 5-MeO-DMT benzoate with a mixture of HPMC 2910 in water produced a 50% wt:wt API to excipient SDD. The spray drying parameters were as below:

Feed Solution

| | |
|---|---|
| Sample Reference | 30130-08-02 |
| Pharmacoat 606 | 1.785 g |
| Metolose 60SH50 | 0.595 g |
| 5-MeO-DMT Benzoate | 2.38 g |
| Water (deionized) | 95.24 g |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |

Yield

| | |
|---|---|
| Yield (%) | 62.7 |

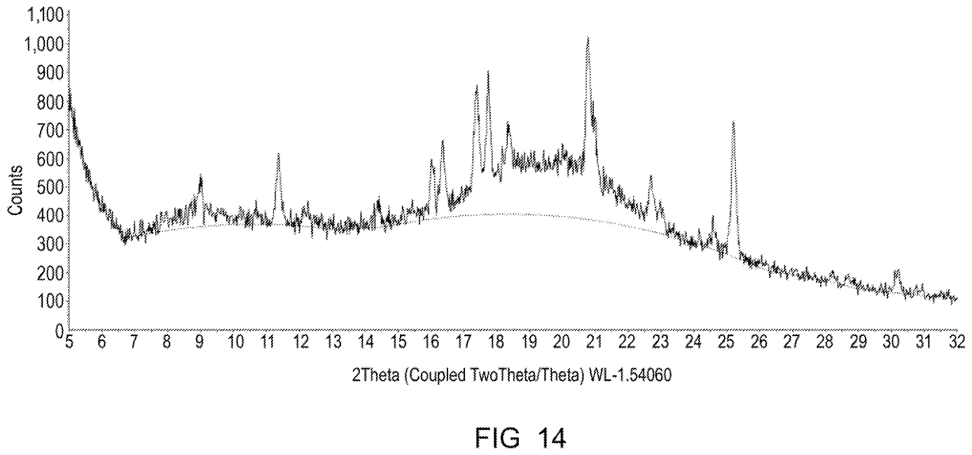

The process for producing the feed solution was as described in Example 5. The SDD produced was partially crystalline (FIG. 14), however, surprisingly the use of 12.5% total loading of metolose led to a significant improvement in yield over the SDD produced in Example 5 (25% metolose).

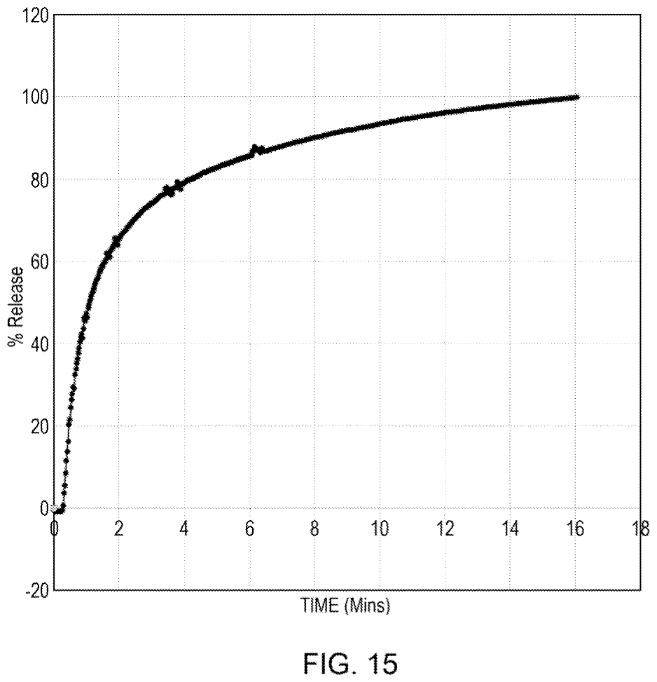

The dissolution profile for the SDD produced can be seen in FIG. 15, this shows that ~80% release has occurred by ~4 minutes.

### Example 7: Spray Drying of 5-MeO-DMT Benzoate Salt with HPMCAS

Spray drying of 5-MeO-DMT benzoate with hydroxypropyl methylcellulose acetate succinate (HPMCAS) M produced a 50% wt:wt API to excipient SDD. HPMCAS is produced in three substitution grades: L, M and H. The spray drying parameters were as below:

Feed Solution

| | |
|---|---|
| Sample Reference | 30120-08-03 |
| HPMCAS M | 2.38 g |
| 5-MeO-DMT Benzoate | 2.38 g |
| Water (deionized) | 47.62 g |
| Acetone | 47.62 g |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 96% |

Yield

| | |
|---|---|
| Yield (%) | 44.7 |

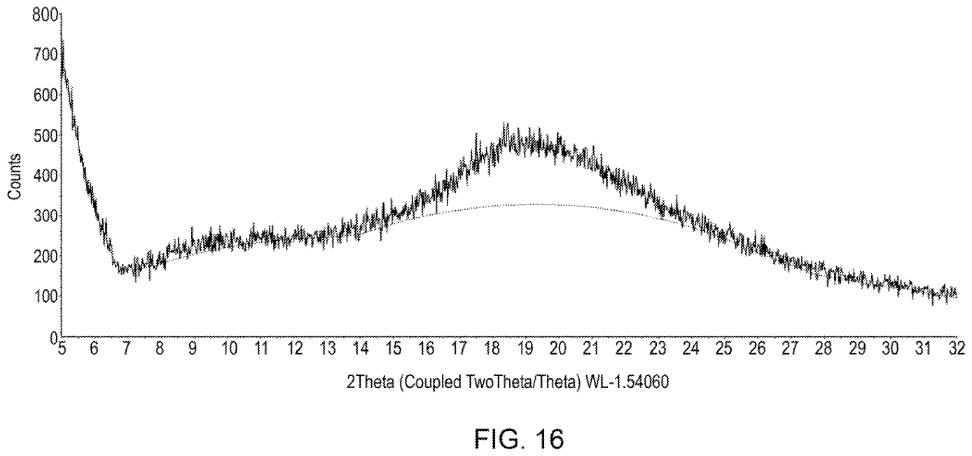

The process for producing the feed solution was as follows: the required mass of acetone was weighed into a 50 mL vial. The required mass of HPMC-AS was added to the acetone whilst stirring and allowed to fully dissolve. Once dissolved the required mass of water was added to the solution followed by API and the vial was then stirred until the API dissolved. Once dissolved the feed solution was spray dried immediately. The SDD produced was state-stable and amorphous (FIG. 16).

### Example 8: Spray Drying of 5-MeO-DMT Benzoate Salt with HPMCAS/Metolose

Spray drying of 5-MeO-DMT benzoate with HPMCAS M and Metolose 60SH50 in water produced a 50% wt:wt API to excipient SDD. The spray drying parameters were as below:

Feed Solution

| | |
|---|---|
| HPMCAS M | 1.19 g |
| Metolose 60SH50 | 1.19 g |
| 5-MeO-DMT Benzoate | 2.38 g |
| Water (deionized) | 47.62 g |
| Acetone | 47.62 g |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Nozzle Power % | 96% |

Yield

| | |
|---|---|
| Yield (%) | 29.2 |

The process for producing the feed solution was as follows: the required mass of acetone was weighed into a 50 mL vial. The required mass of HPMC-AS was added to the acetone whilst stirring and allowed to fully dissolve. Once dissolved the required mass of water was added to the solution followed by HPMC, the vial was stirred overnight to dissolve. Once dissolved the API was added and stirred, once dissolved the feed solution was spray dried immediately.

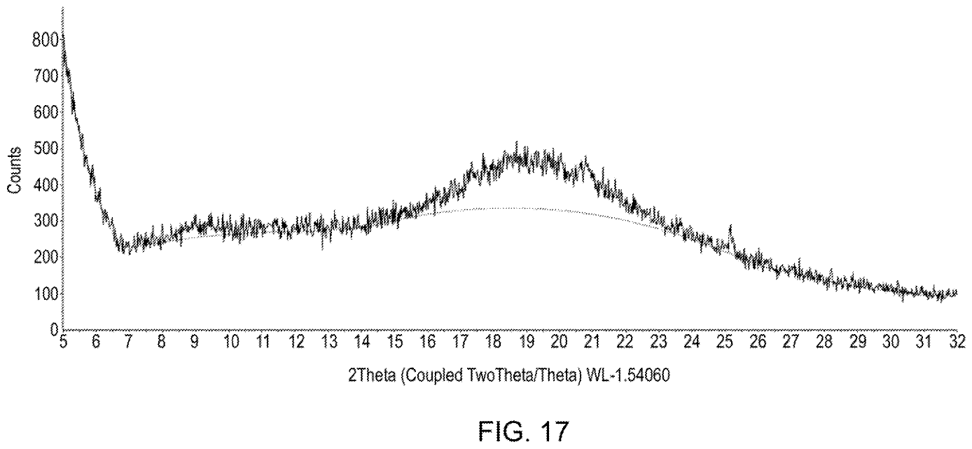

The SDD produced was predominantly amorphous (FIG. 17), however, there was some difficulty in spray drying viscous solutions containing HPMC-AS.

### Example 9: Spray Drying of 5-MeO-DMT Benzoate Salt with HPMCAS/Metolose

Spray drying of 5-MeO-DMT benzoate with HPMCAS M and Metolose 60SH50 in water produced a 50% wt:wt API to excipient SDD. The spray drying parameters were as below:

Feed Solution

| | |
|---|---|
| HPMCAS M | 1.785 g |
| Metolose 60SH50 | 1.595 g |
| 5-MeO-DMT benzoate | 2.38 g |
| Water (deionized) | 47.62 gg |
| Acetone | 47.62 g |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Nozzle Power % | 96% |

Yield

| | |
|---|---|
| Yield (%) | 27.2 |

The process for producing the feed solution was as follows: the required mass of acetone was weighed into a 50 mL vial. The required mass of HPMC-AS was added to the acetone whilst stirring and allowed to fully dissolve. Once dissolved the required mass of water was added to the solution followed by HPMC, the vial was stirred overnight to dissolve. Once dissolved the API was added and stirred, once dissolved the feed solution was spray dried immediately.

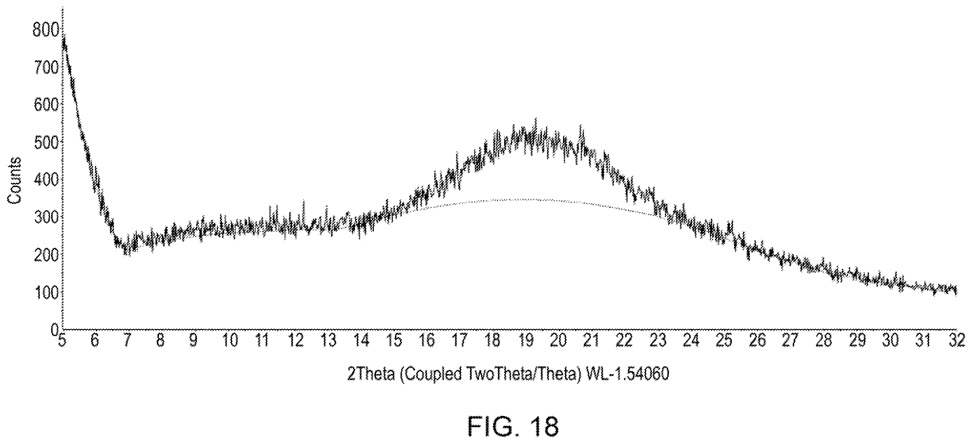

The SDD produced was predominantly amorphous (FIG. 18), however, there was some difficulty in spray drying viscous solutions containing HPMC-AS.

### Example 10: Spray Drying of 5-MeO-DMT Benzoate Salt with HPMC Mixtures

Spray drying of 5-MeO-DMT benzoate with a mixture of HPMC 2910 in water produced a 10% wt:wt API to excipient SDD. The spray drying parameters were as below:

SDD formulation

| | |
|---|---|
| Sample Reference | 3815-02-01 |
| Pharmacoat 606 | 67.5% |
| Metolose 60SH50 | 22.5% |
| 5-MeO-DMT Benzoate | 10% |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |
| Feed Sock % solids | |

Yield

| | |
|---|---|
| Yield (%) | 58% |

The process for spray drying the feed solution was as follows: the required mass of water was weighed into a 50 mL vial. The required mass of HPMC and Metolose were added to the water whilst stirring and allowed to fully dissolve. Once dissolved the required mass of API was transferred into the solution and allowed to dissolve. Once dissolved the feed solution was spray dried immediately.

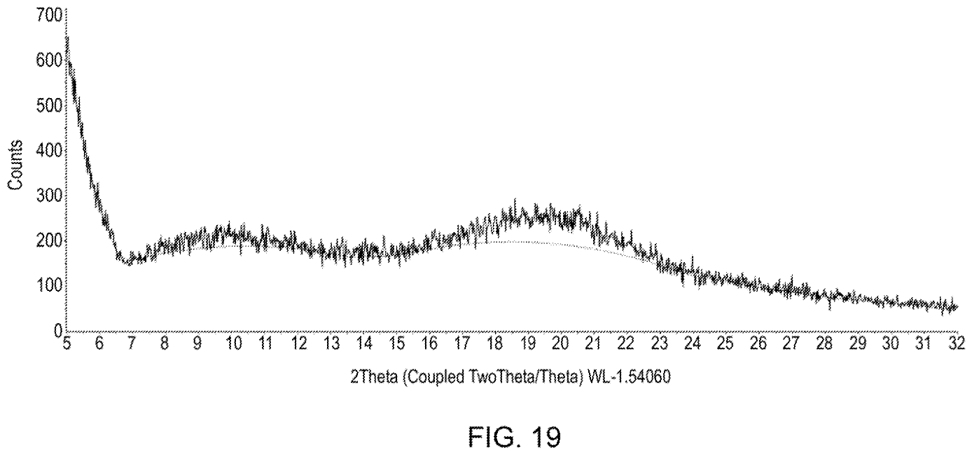

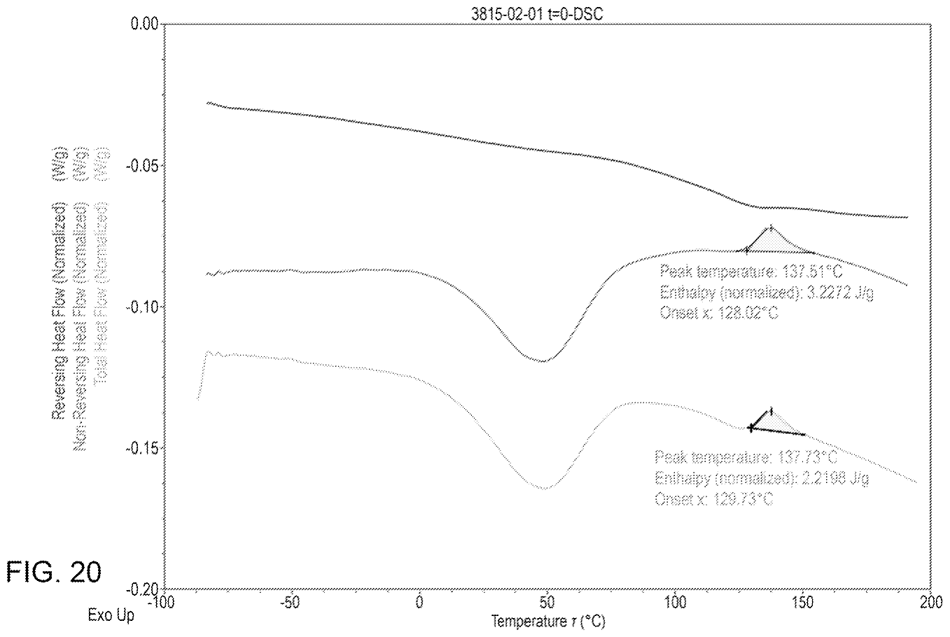

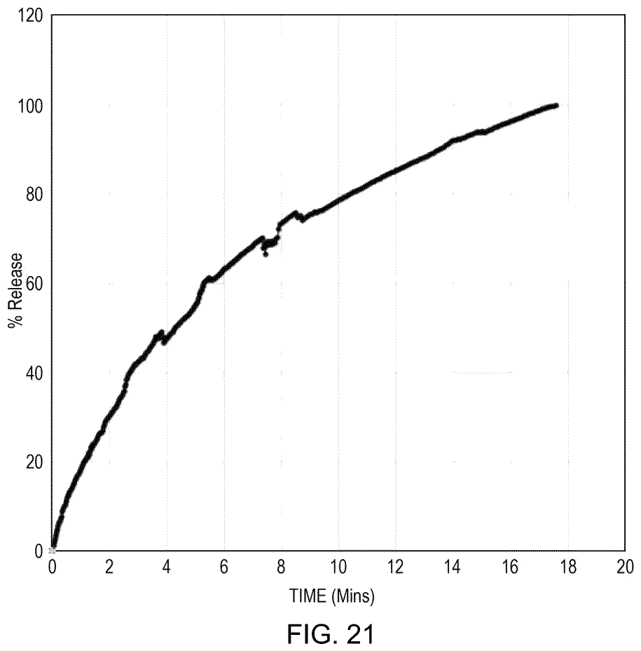

The SDD produced was stable and amorphous by XRPD (FIG. 19) and DSC (FIG. 20). The dissolution profile of the SDD (FIG. 21) shows that ~80% release has occurred by ~10 minutes, compared with the ~4 minutes for the SDD of Example 6.

### Example 11: Spray Drying of 5-MeO-DMT Benzoate Salt with HPMC Mixtures

Spray drying of 5-MeO-DMT benzoate with a mixture of HPMC 2910 in water produced a 30% wt:wt API to excipient SDD. The spray drying parameters were as below:

SDD formulation

| | |
|---|---|
| Sample Reference | 3815-02-02 |
| Pharmacoat 606 | 52.5% |
| Metolose 60SH50 | 17.5% |
| 5-MeO-DMT Benzoate | 30% |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |
| Feed Sock % solids | |

Yield

| | |
|---|---|
| Yield (%) | 60% |

The process for spray drying the feed solution was as follows: the required mass of water was weighed into a 50 mL vial. The required mass of HPMC and metolose were added to the water whilst stirring and allowed to fully dissolve. Once dissolved the required mass of API was transferred into the solution and allowed to dissolve. Once dissolved the feed solution was spray dried immediately.

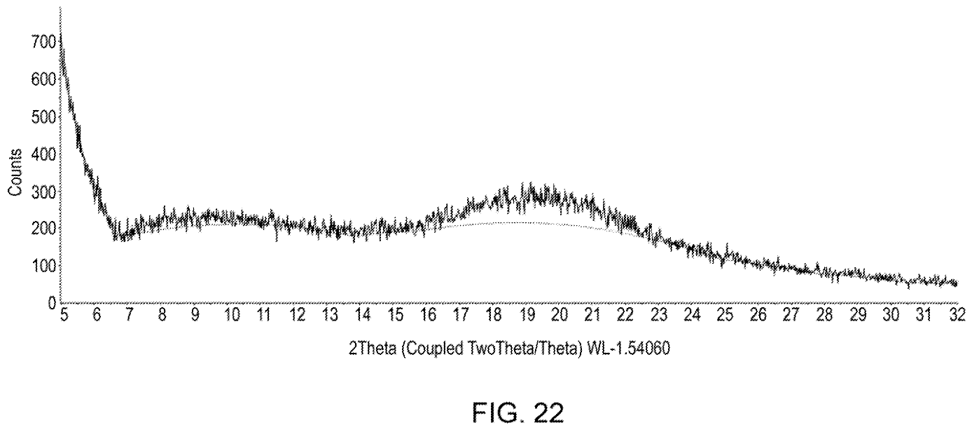

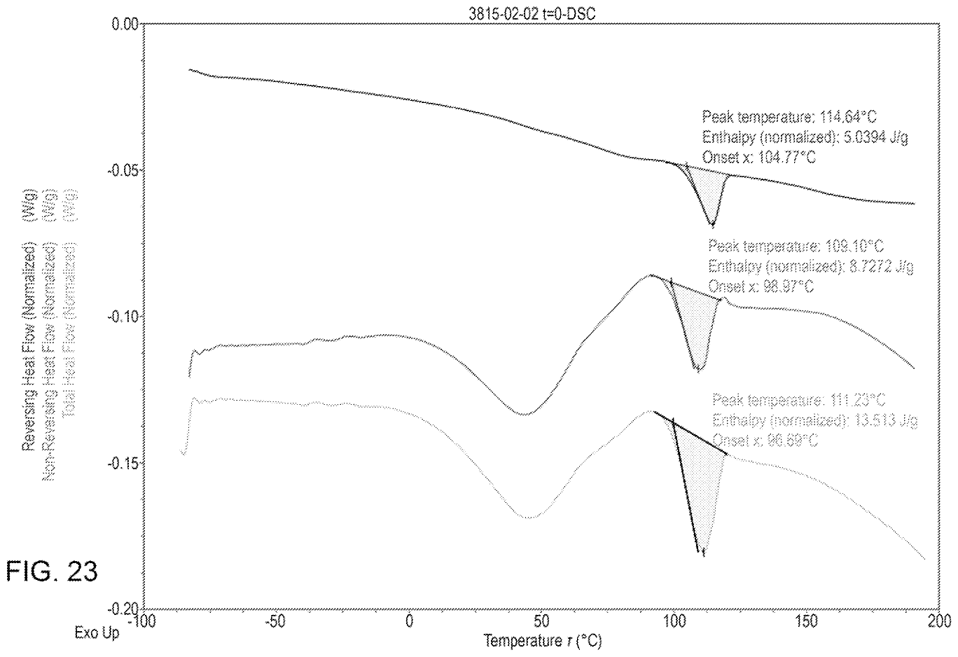

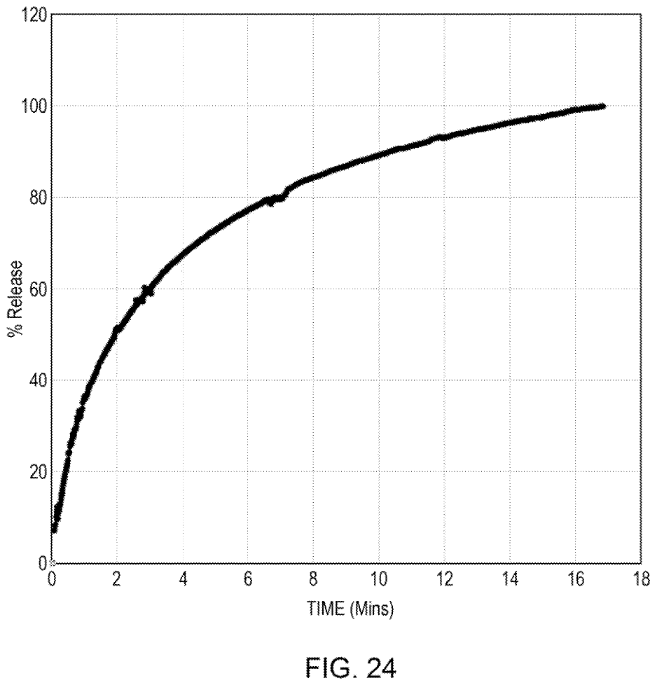

The SDD produced was stable and amorphous by XRPD (FIG. 22) and DSC (FIG. 23). The dissolution profile of the SDD (FIG. 24) shows that ~80% release has occurred by ~6.5 minutes, compared with ~4 minutes for the SDD of Example 6 and ~10 minutes for the SDD of Example 10.

### Example 12: Spray Drying of 5-MeO-DMT Benzoate Salt with HPMC Mixtures

Spray drying of 5-MeO-DMT benzoate salt with a mixture of HPMC 2910 in water produced a 50% wt:wt API to excipient SDD. The spray drying parameters were as below:

SDD formulation

| | |
|---|---|
| Example Reference | 3815-02-02 |
| Pharmacoat 606 | 37.5% |
| Metolose 60SH50 | 12.5% |
| 5-MeO-DMT Benzoate | 50% |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |
| Feed Sock % solids | |

Yield

| | |
|---|---|
| Yield (%) | 52% |

The spray drying process was as follows: the required mass of water was weighed into a 50 mL vial. The required mass of HPMC and metolose were added to the water whilst stirring and allowed to fully dissolve. Once dissolved the required mass of API was transferred into the solution and allowed to dissolve. Once dissolved the feed solution was spray dried immediately.

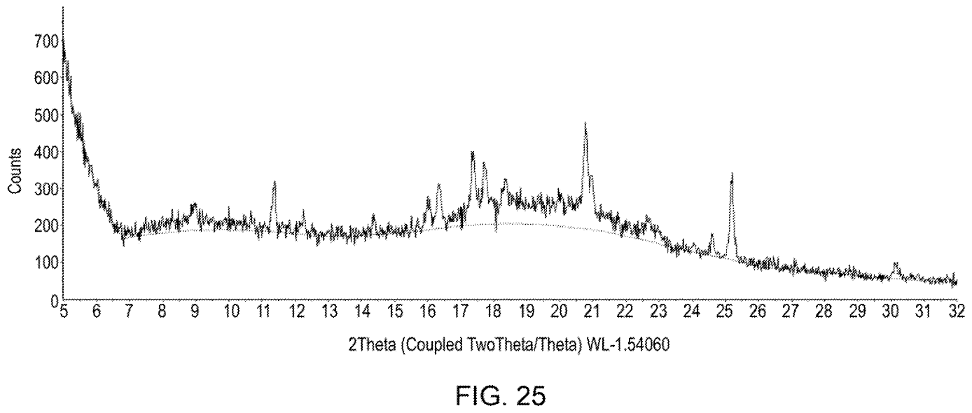

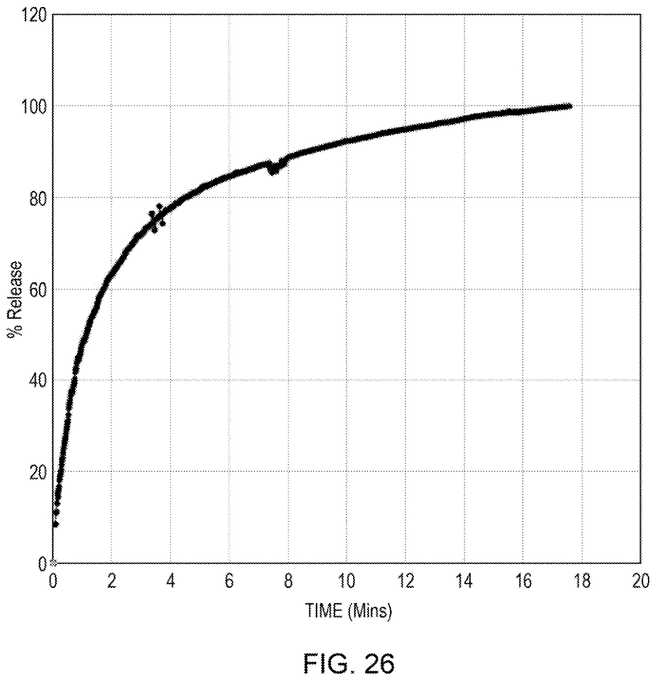

The SDD produced was partially crystalline (FIG. 25) with a dissolution profile (FIG. 26) that shows that ~80% release has occurred by ~4.5 minutes, compared with ~4 minutes for the SDD of Example 6, ~10 minutes for the SDD of Example 10 and ~6.5 minutes for the SDD of Example 11.

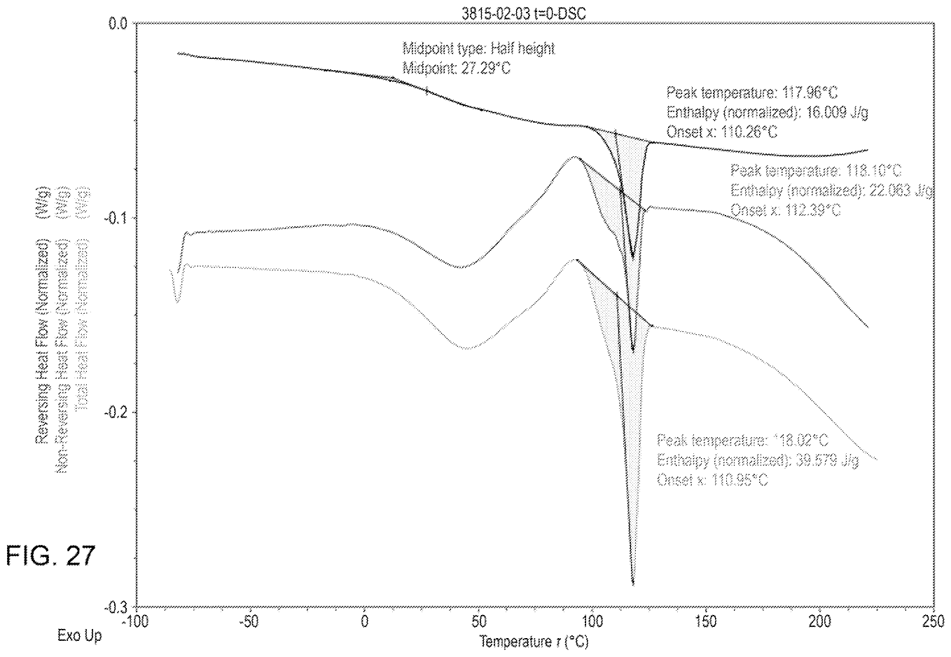

The DSC thermogram for the SDD (FIG. 27) shows a small peak at ~140° C. indicating the presence of crystalline API. Such a peak is not seen in the equivalent HBr or HCl salt pharmaceutical compositions or formulations.

### Example 13: Spray Drying of 5-MeO-DMT Benzoate Salt with HPMC Mixtures

Spray drying of 5-MeO-DMT benzoate salt with a mixture of HPMC 2910 and sorbitol in water produced a 10% wt:wt API to excipient SDD. The spray drying parameters were as below:

SDD formulation

| | |
|---|---|
| Sample Reference | 3815-02-04 |
| Pharmacoat 606 | 65.25% |
| Metolose 60SH50 | 21.75% |
| Sorbitol M | 3% |
| 5-MeO-DMT Benzoate | 10% |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |
| Feed Sock % solids | |

Yield

| | |
|---|---|
| Yield (%) | 61% |

The spray drying process was as follows: the required mass of water was weighed into a 50 mL vial. The required mass of HPMC and metolose were added to the water whilst stirring and allowed to fully dissolve. Once dissolved the required mass of API and sorbitol were transferred into the solution and allowed to dissolve. Once dissolved the feed solution was spray dried immediately.

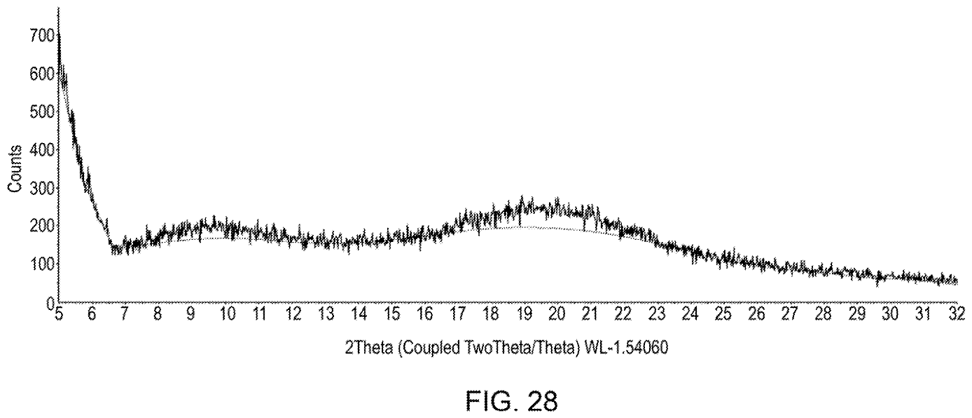

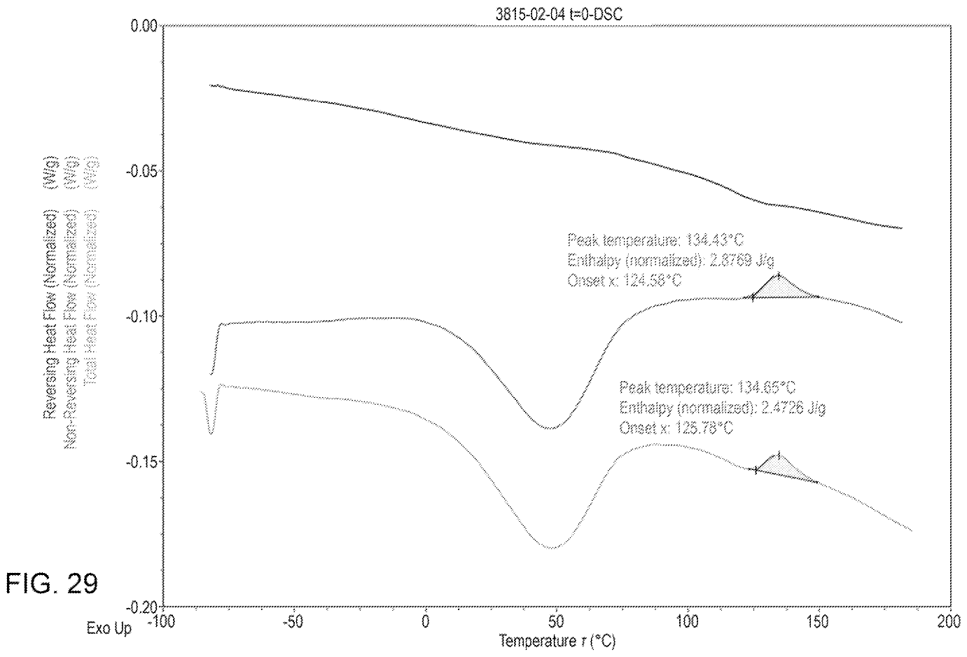

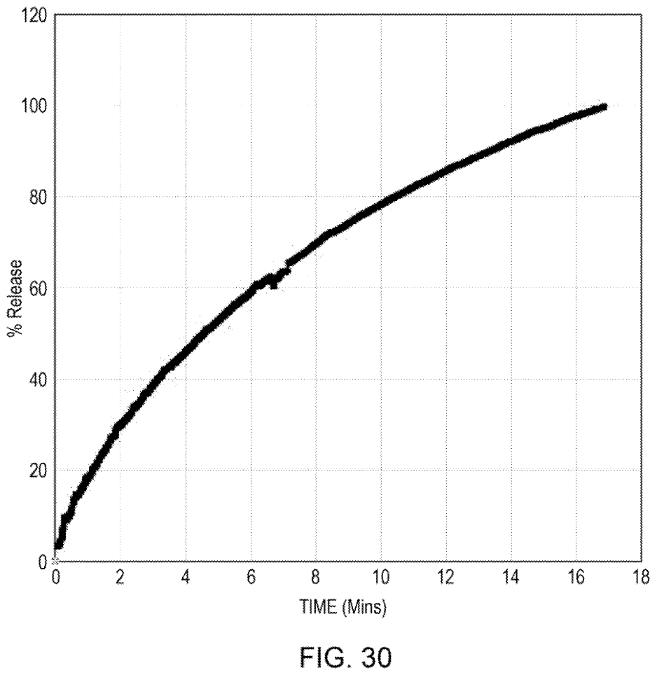

The SDD produced was stable and amorphous, as shown in FIGS. 28 and 29, and the yield had improved over that which was observed for the SDD of Example 12. The dissolution profile (FIG. 30) shows that ~80% release has occurred by ~10 minutes compared with ~4 minutes for the SDD of Example 6, ~10 minutes for the SDD of Example 10, ~6.5 minutes for the SDD of Example 11 and ~4.5 minutes for Example 12.

### Example 14: Spray Drying of 5-MeO-DMT Benzoate Salt with HPMC Mixtures

Spray drying of 5-MeO-DMT benzoate salt with a mixture of HPMC 2910 and sorbitol in water produced a 30% wt:wt API to excipient SDD. The spray drying parameters were as below:

SDD formulation

| | |
|---|---|
| Sample Reference | 3815-02-05 |
| Pharmacoat 606 | 50.25% |
| Metolose 60SH50 | 16.75% |
| Sorbitol M | 3% |
| 5-MeO-DMT Benzoate | 30% |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |
| Feed Sock % solids | 3.1 |

Yield

| | |
|---|---|
| Yield (%) | 78% |

The spray drying process was as follows: the required mass of water was weighed into a 50 mL vial. The required mass of HPMC and metolose were added to the water whilst stirring and allowed to fully dissolve.

Once dissolved the required mass of API and sorbitol was transferred into the solution and allowed to dissolve. Once dissolved the feed solution was spray dried immediately.

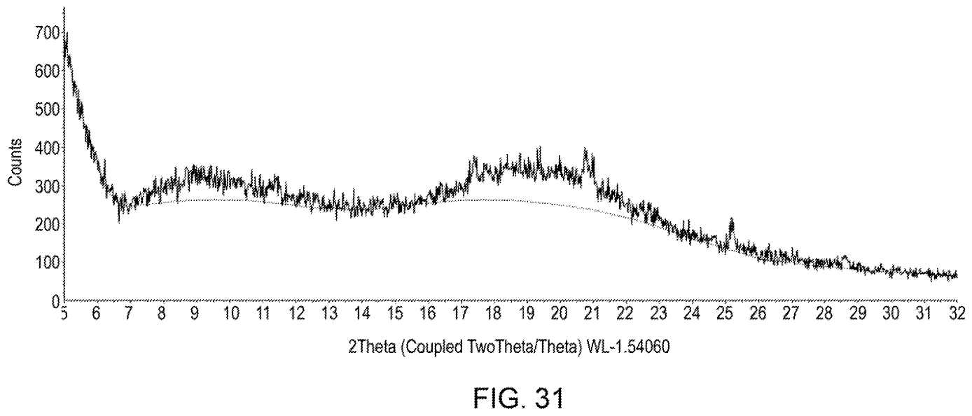

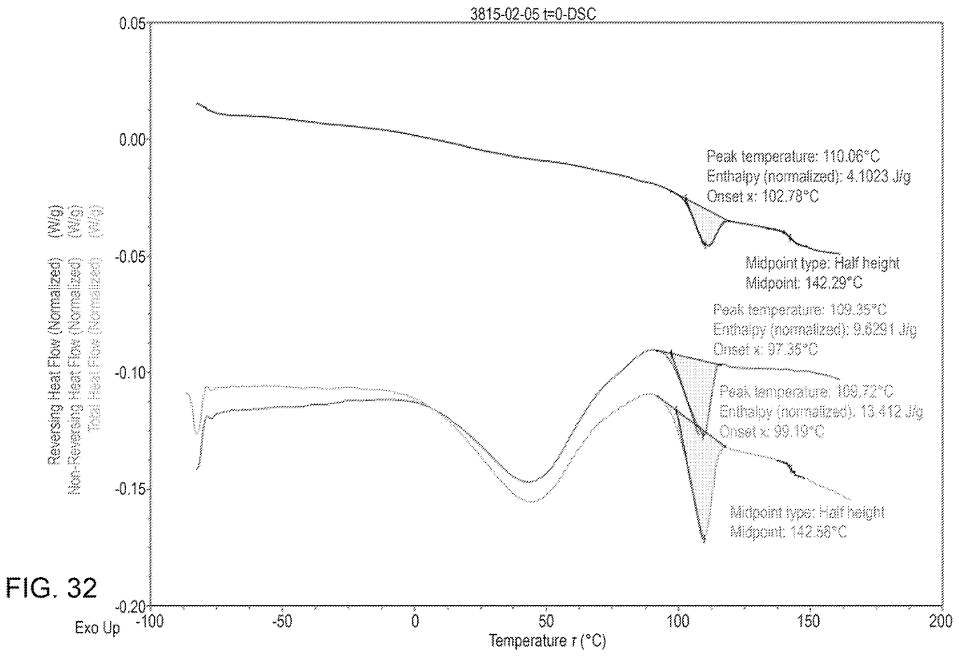

The SDD produced was amorphous (FIG. 31) and similar to that produced in Example 12, however, the yield was significantly improved, 52% vs 78%. The DSC thermogram shown in FIG. 32.

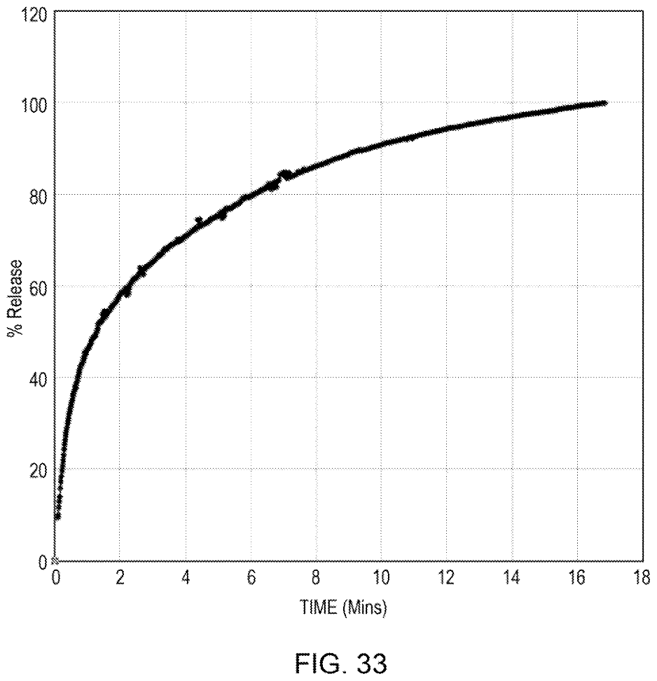

The dissolution profile, shown in FIG. 33, shows that for the SDD of Example 14~80% release has occurred by ~6 minutes, compared with ~4 minutes for the SDD of Example 6, ~10 minutes for the SDD of Example 10, ~6.5 minutes for the SDD of Example 11, ~4.5 minutes for Example 12 and ~10 minutes for the SDD of Example 13.

The inclusion of sorbitol results in an improved yield with no impact on the dissolution rate or stability.

### Example 15: Spray Drying of 5-MeO-DMT Benzoate Salt with HPMC Mixtures

Spray drying of 5-MeO-DMT benzoate salt with a mixture of HPMC 2910 and sorbitol in water produced a 50% wt:wt API to excipient SDD. The spray drying parameters were as below:

SDD formulation

| | |
|---|---|
| Sample Reference | 3815-02-06 |
| Pharmacoat 606 | 35.25% |
| Metolose 60SH50 | 11.75% |
| Sorbitol M | 3% |
| 5-MeO-DMT Benzoate | 50% |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |
| Feed Sock % solids | 4.8 |

Yield

| | |
|---|---|
| Yield (%) | 70% |

The spray drying process was as follows: the required mass of water was weighed into a 50 mL vial. The required mass of HPMC and metolose were added to the water whilst stirring and allowed to fully dissolve. Once dissolved the required mass of API and sorbitol was transferred into the solution and allowed to dissolve. Once dissolved the feed solution was spray dried immediately.

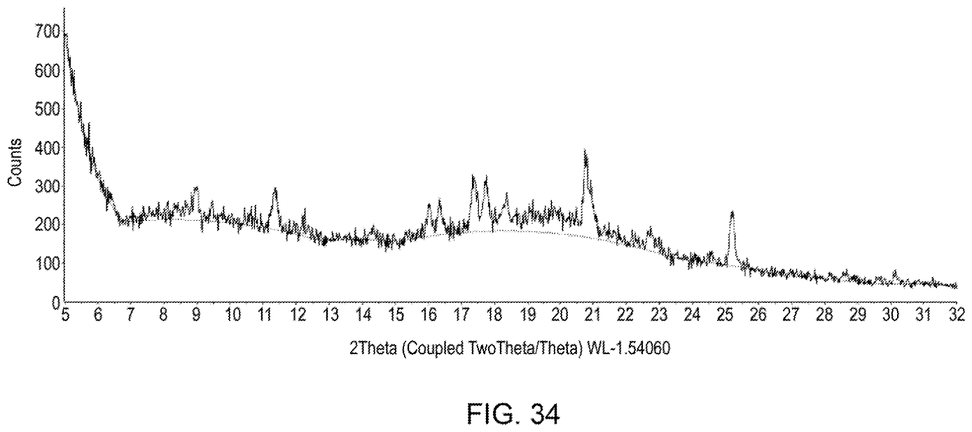

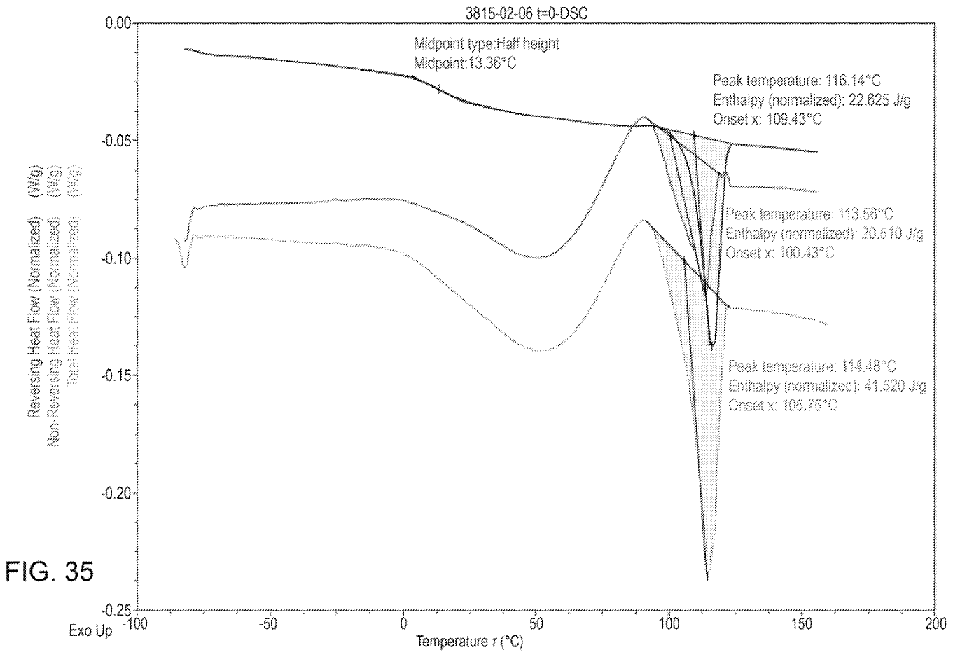

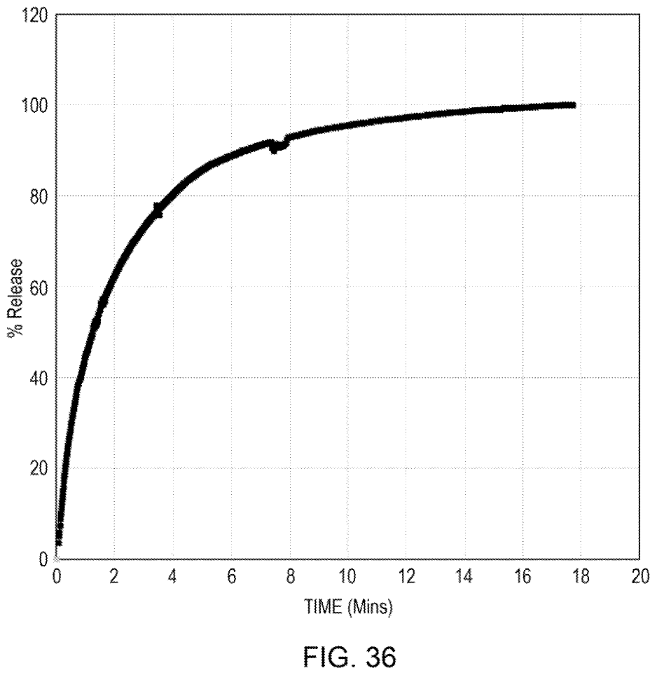

The SDD produced was partially crystalline (FIG. 34) and the DSC thermogram and dissolution profile of this SDD can be seen in FIGS. 35 and 36. The dissolution profile, shown in FIG. 33, shows that for the SDD of Example 15~80% release has occurred by ~4 minutes, compared with ~4 minutes for the SDD of Example 6, ~10 minutes for the SDD of Example 10, ~6.5 minutes for the SDD of Example 11, ~4.5 minutes for Example 12, ~10 minutes for the SDD of Example 13 and ~6 minutes for the SDD of Example 14.

### Example 16: Spray Drying of 5-MeO-DMT Hydrobromide Salt with HPMC Mixtures

Spray drying of 5-MeO-DMT hydrobromide salt with a mixture of HPMC 2910 and sorbitol in water produced a 50% wt:wt API to excipient SDD. The spray drying parameters were as below:

SDD formulation

| | |
|---|---|
| Sample Reference | 3815-37-01 |
| Pharmacoat 606 | 35.25% |
| Metolose 60SH50 | 11.75% |
| Sorbitol M | 3% |
| 5-MeO-DMT hydrobromide | 50% |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |
| Feed Sock % solids | 4.8 |

Yield

| | |
|---|---|
| Yield (%) | 81.75% |

The spray drying process was as follows: the required mass of water was weighed into a 50 mL vial. The required mass of HPMC and metolose were added to the water whilst stirring and allowed to fully dissolve. Once dissolved the required mass of API and sorbitol was transferred into the solution and allowed to dissolve. Once dissolved the feed solution was spray dried immediately.

The SDD produced was stable and amorphous, unlike that of Example 15, with a dissolution profile similar to that of Example 15. Additionally, the yield for this SDD was higher than that of the SDD of Example 15.

### Example 17: Spray Drying of 5-MeO-DMT Hydrochloride Salt with HPMC Mixtures

Spray drying of 5-MeO-DMT hydrochloride salt with a mixture of HPMC 2910 and sorbitol in water produced a 50% wt:wt API to excipient SDD. The spray drying parameters were as below:

SDD formulation

| | |
|---|---|
| Sample Reference | 3815-37-02 |
| Pharmacoat 606 | 35.25% |
| Metolose 60SH50 | 11.75% |
| Sorbitol M | 3% |
| 5-MeO-DMT hydrochloride | 50% |

Spray Drying Parameters

| | |
|---|---|
| Instrument | Procept |
| Nozzle | Ultrasonic |
| Inlet Flow (m³/min) | 0.7 |
| Inlet Temperature (°C) | 125 |
| Cyclone Gas Pressure (bar) | 0.5 |
| Pump Speed (RPM) | 60 (2 g per minute) |
| Nozzle Power % | 98% |
| Feed Sock % solids | 4.8 |

Yield

| | |
|---|---|
| Yield (%) | 78.4%% |

The spray drying process was as follows: the required mass of water was weighed into a 50 mL vial. The required mass of HPMC and metolose were added to the water whilst stirring and allowed to fully dissolve. Once dissolved the required mass of API and sorbitol was transferred into the solution and allowed to dissolve. Once dissolved the feed solution was spray dried immediately.

The SDD produced was stable and amorphous, unlike the SDD produced in Example 15. The dissolution profile was similar to the SDD of Example 15.

### Example 18: Lyophilisation of HBr Salt with PVP

Lyophilisation of Hydrobromide salt with PVP in water to produce a 50% wt:wt API lyophilized dispersion.

Feed Solution

| | |
|---|---|
| Sample Reference | |
| PVP | 250 mg |
| 5-MeO-DMT HBr | 250 mg |
| Water (deionized) | 10 ml |

Lyophilization Profile

| Temp | Ramp | Hold | | Vacuum |
|---|---|---|---|---|
| +20 °C | | 10 | mins | |
| −50 °C | 140 mins | 240 | mins | |
| −50 °C | — | 10 | mins | 500 |
| −30 °C | 40 mins | 900 | mins | 500 |
| −30 °C | — | 8100 | mins | 500 |
| +20 °C | 100 mins | 900 | mins | 100 |
| +20 | — | 500 | mins | 100 |

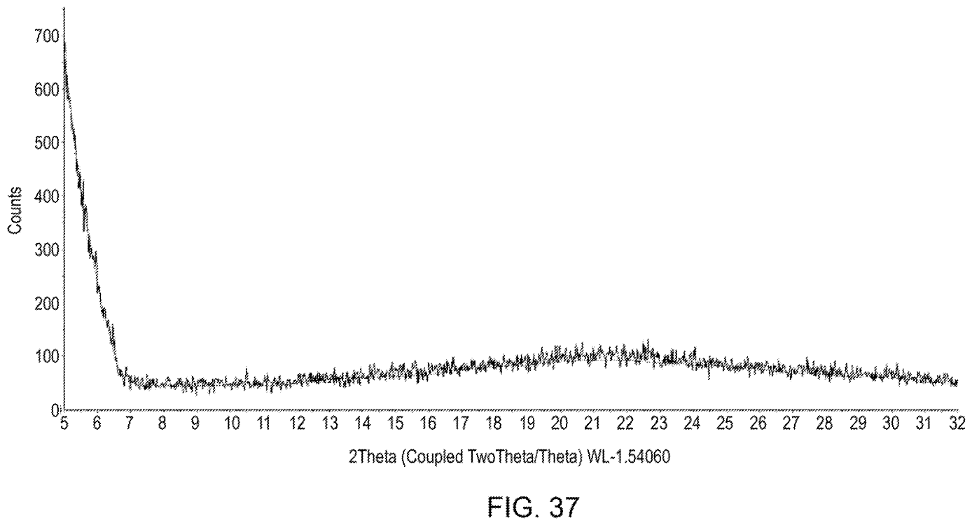

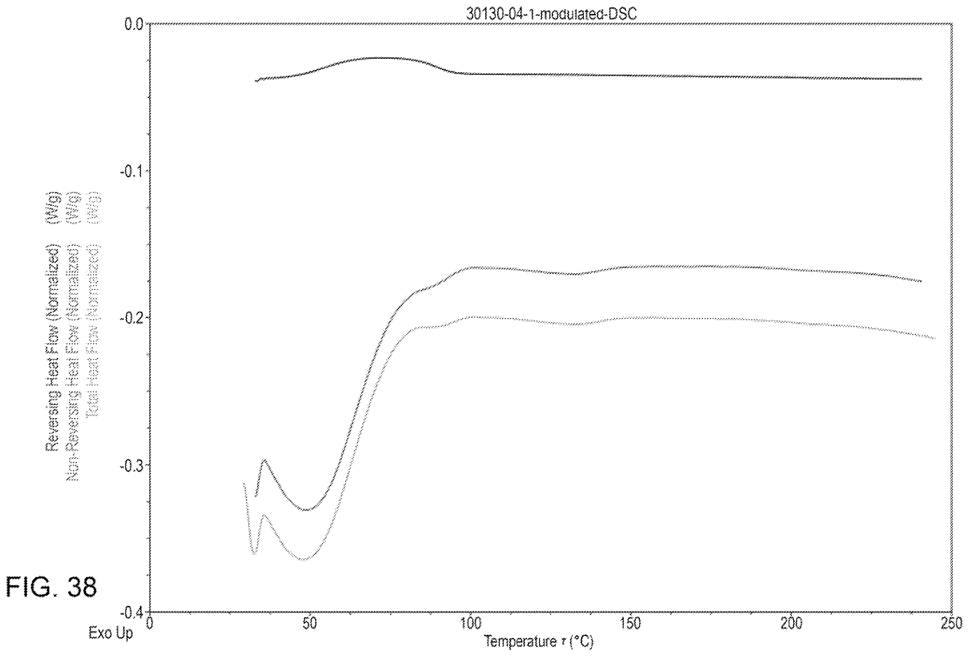

Demonstrates that the HBr at 50% loading in PVP will produce an amorphous lyophilized product. FIG. 37 shows an XRPD of the lyophilized dispersion of Example 18. FIG. 38 shows a DSC thermogram for the lyophilized dispersion of Example 18.

### Example 19: Lyophilisation of HBr Salt with Lactose Monohydrate

Lyophilisation of Hydrobromide salt with lactose monohydrate in water to produce a 50% wt:wt API lyophilized dispersion.

Feed Solution

| | |
|---|---|
| Sample Reference | 30130-04-3 |
| Lactose monohydrate | 250 mg |
| 5-MeO-DMT HBr | 250 mg |
| Water (deionized) | 10 ml |

Lyophilisation Profile

| Temp | Ramp | Hold | | Vacuum |
|---|---|---|---|---|
| +20 °C | | 10 | mins | |
| −50 °C | 140 mins | 240 | mins | |
| −50 °C | — | 10 | mins | 500 |
| −30 °C | 40 mins | 900 | mins | 500 |
| −30 °C | — | 8100 | mins | 500 |
| +20 °C | 100 mins | 900 | mins | 100 |
| +20 | — | 500 | mins | 100 |

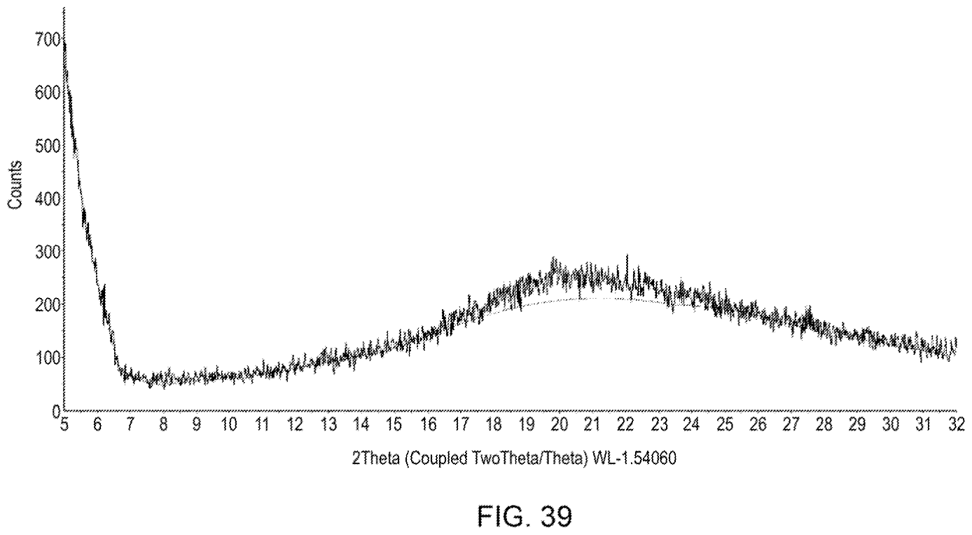

Demonstrates that the HBr at 50% loading in lactose monohydrate will produce an amorphous lyophilized product. FIG. 39 shows an XRPD of the lyophilized dispersion of Example 19. FIG. 40 shows a DSC thermogram for the lyophilized dispersion of Example 19.

### Example 20: Lyophilisation of HBr Salt with Trehalose

Lyophilisation of Hydrobromide salt with trehalose in water to produce a 50% wt:wt API lyophilized dispersion.

Feed Solution

| | |
|---|---|
| Sample Reference | 30130-04-4 |
| trehalose | 250 mg |
| 5-MeO-DMT HBr | 250 mg |
| Water (deionized) | 10 ml |

Lyophilisation Profile

| Temp | Ramp | Hold | | Vacuum |
|---|---|---|---|---|
| +20 °C | | 10 | mins | |
| −50 °C | 140 mins | 240 | mins | |
| −50 °C | — | 10 | mins | 500 |
| −30 °C | 40 mins | 900 | mins | 500 |
| −30 °C | — | 8100 | mins | 500 |
| +20 °C | 100 mins | 900 | mins | 100 |
| +20 | — | 500 | mins | 100 |

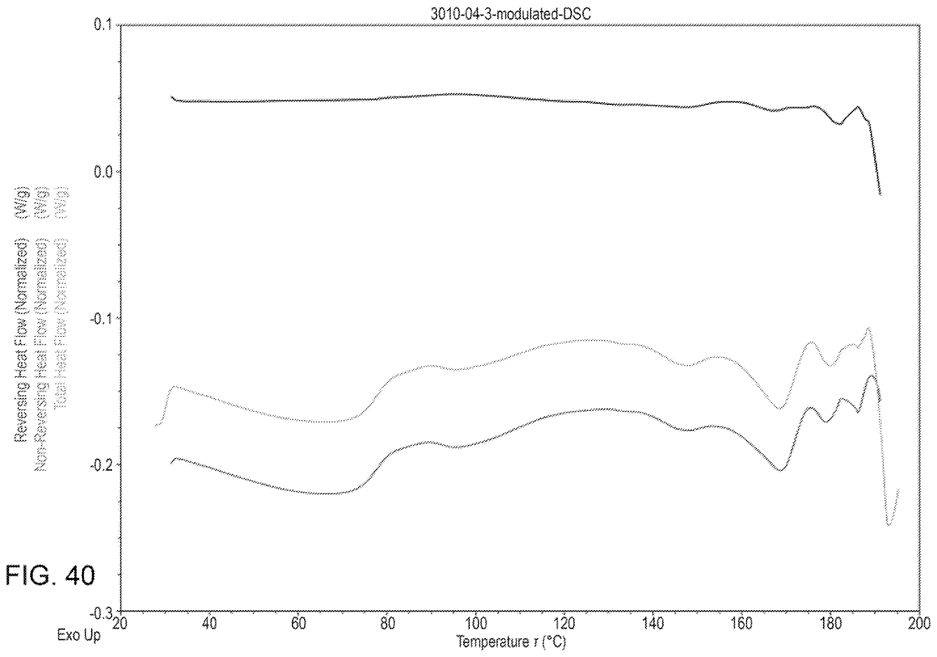

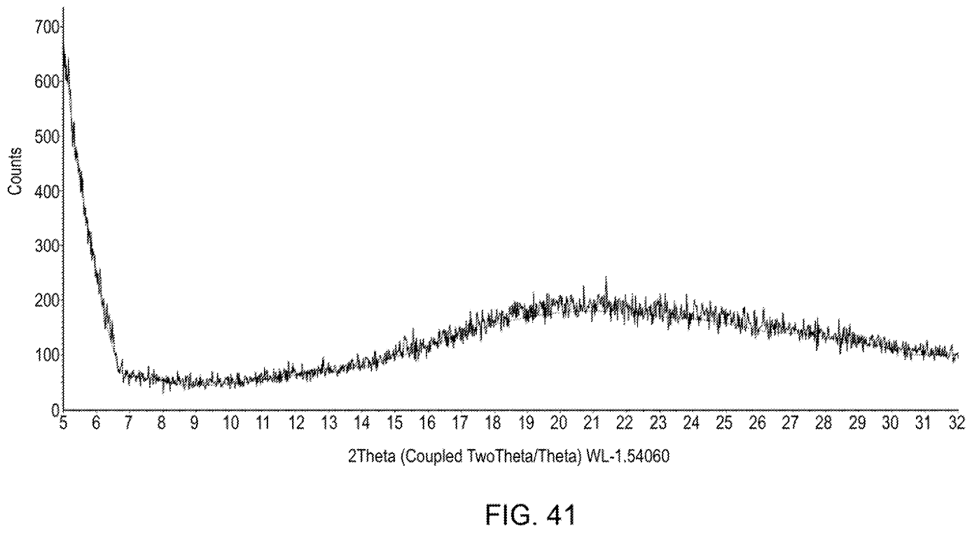

Demonstrates that the HBr at 50% loading in trehalose will produce an amorphous lyophilized product. FIG. 41 shows an XRPD of the lyophilized dispersion of Example 20. FIG. 42 shows a DSC thermogram for the lyophilized dispersion of Example 20.

### Example 21: Lyophilisation of Oxalate Salt with Trehalose

Lyophilisation of Oxalate salt with trehalose in water to produce a 50% wt:wt API lyophilized dispersion.

Feed Solution

| | |
|---|---|
| Sample Reference | 30130-04-5 |
| Trehalose | 250 mg |
| 5-MeO-DMT oxalate | 250 mg |
| Water (deionized) | 10 ml |

Lyophilisation Profile

| Temp | Ramp | Hold | | Vacuum |
|---|---|---|---|---|
| +20 °C | | 10 | mins | |
| −50 °C | 140 mins | 240 | mins | |
| −50 °C | — | 10 | mins | 500 |
| −30 °C | 40 mins | 900 | mins | 500 |
| −30 °C | — | 8100 | mins | 500 |
| +20 °C | 100 mins | 900 | mins | 100 |
| +20 | — | 500 | mins | 100 |

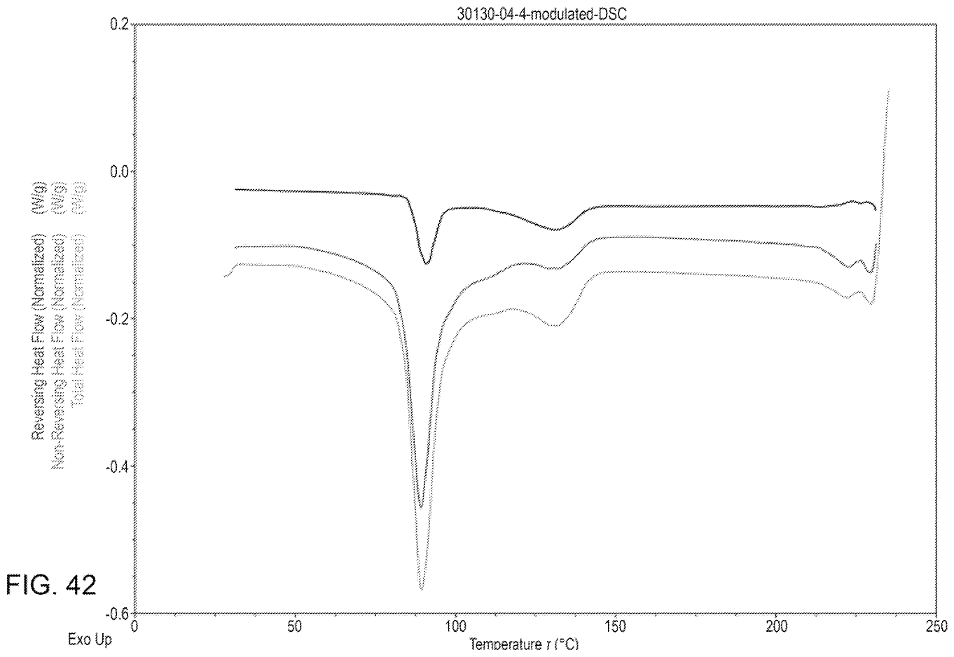

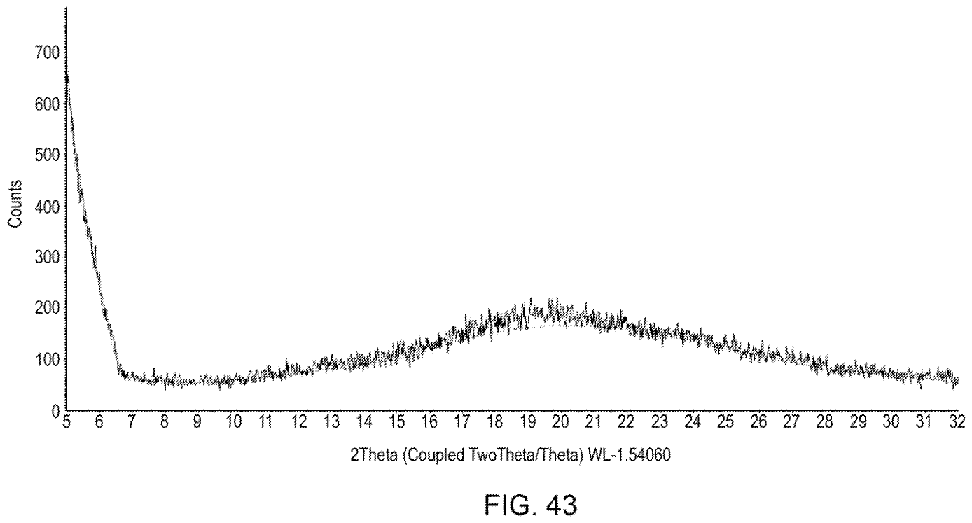

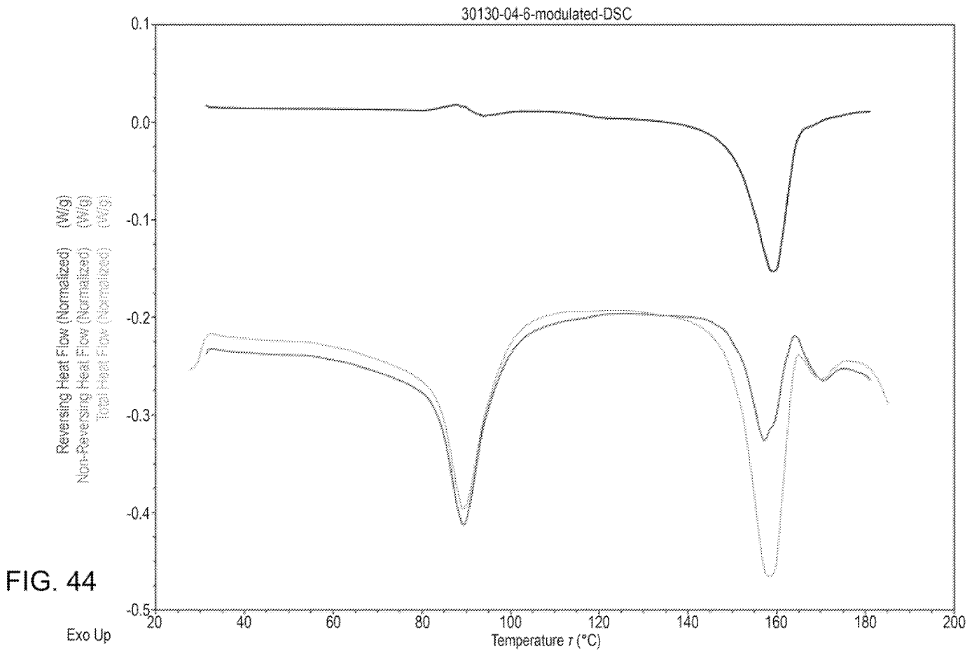

Demonstrates that the Oxalate at 50% loading in trehalose will produce an amorphous lyophilized product. FIG. 43 shows an XRPD of the lyophilized dispersion of Example 21. FIG. 44 shows a DSC thermogram for the lyophilized dispersion of Example 21.

### Example 22: Lyophilisation of HBr Salt with Mannitol and Trehalose

Lyophilisation of HBr salt with mannitol/trehalose in water to produce a 50% wt:wt API lyophilized dispersion.

Feed Solution

| | |
|---|---|
| Sample Reference | 3815-24-02 |
| Mannitol | 150 mg |
| Trehalose | 50 mg |
| 5-MeO-DMT HBr | 200 mg |
| Water (deionized) | 10 ml |

Lyophilisation Profile

| Temp | Ramp | Hold | | Vacuum |
|---|---|---|---|---|
| +20 °C | | 10 | mins | |
| −50 °C | 140 mins | 240 | mins | |
| −50 °C | — | 10 | mins | 500 |
| −30 °C | 40 mins | 900 | mins | 500 |
| −30 °C | — | 8100 | mins | 500 |
| +20 °C | 100 mins | 900 | mins | 100 |
| +20 | — | 500 | mins | 100 |

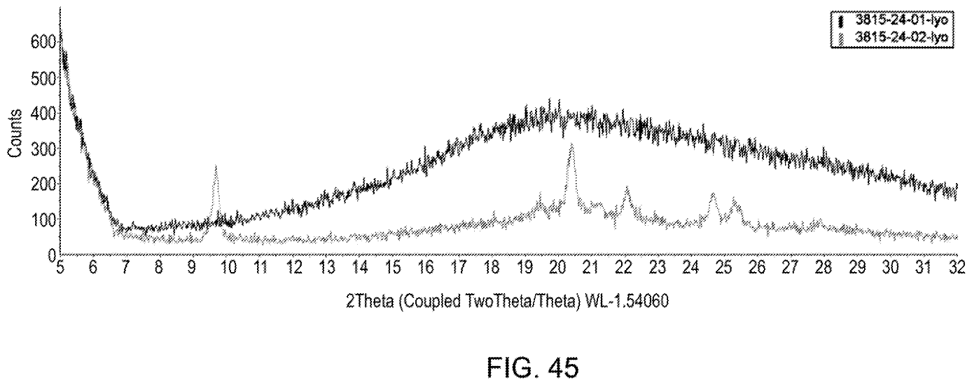

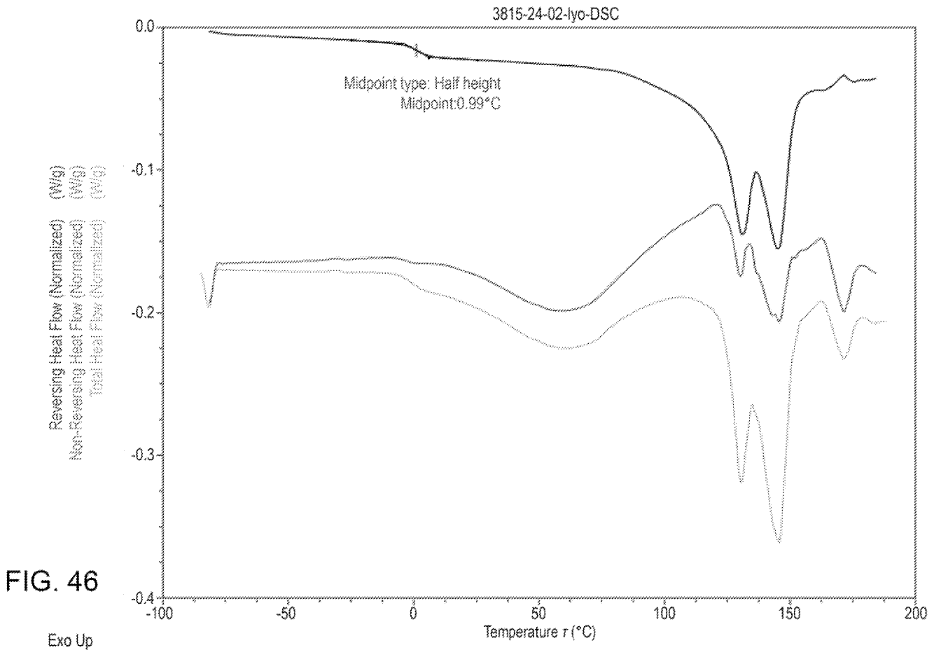

Demonstrates that the HBr at 50% loading in mannitol/trehalose does not produce an amorphous lyophilized product. FIG. 45 shows an XRPD of the lyophilized dispersion of Example 22. FIG. 46 shows a DSC thermogram for the lyophilized dispersion of Example 22.

### Example 23: Lyophilisation of HBr Salt with Mannitol and Trehalose

Lyophilisation of HBr salt with mannitol/trehalose in water to produce a 50% wt:wt API lyophilized dispersion.

Feed Solution

| | |
|---|---|
| Sample Reference | 3815-24-02 |
| Mannitol | 150 mg |
| Trehalose | 50 mg |
| 5MeO DMT HBr | 200 mg |
| Water (deionized) | 10 ml |

Lyophilisation Profile

| Temp | Ramp | Hold | | Vacuum |
|---|---|---|---|---|
| +20 °C | | 10 | mins | |
| −50 °C | 140 mins | 240 | mins | |
| −50 °C | — | 10 | mins | 500 |
| −30 °C | 40 mins | 900 | mins | 500 |
| −30 °C | — | 8100 | mins | 500 |
| +20 °C | 100 mins | 900 | mins | 100 |
| +20 | — | 500 | mins | 100 |

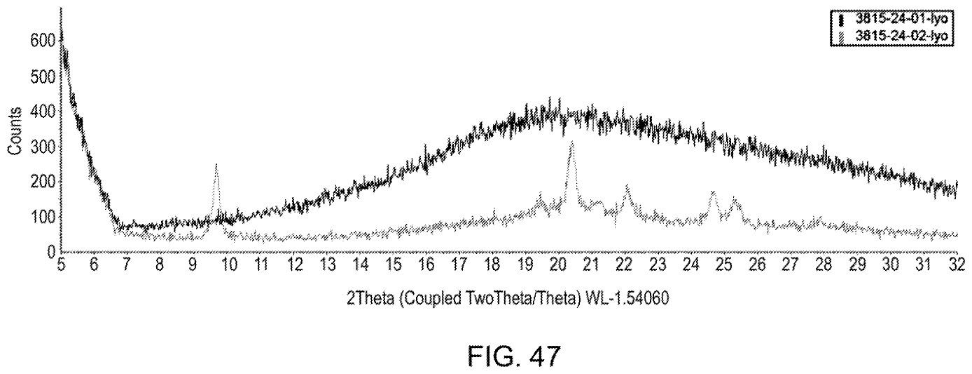

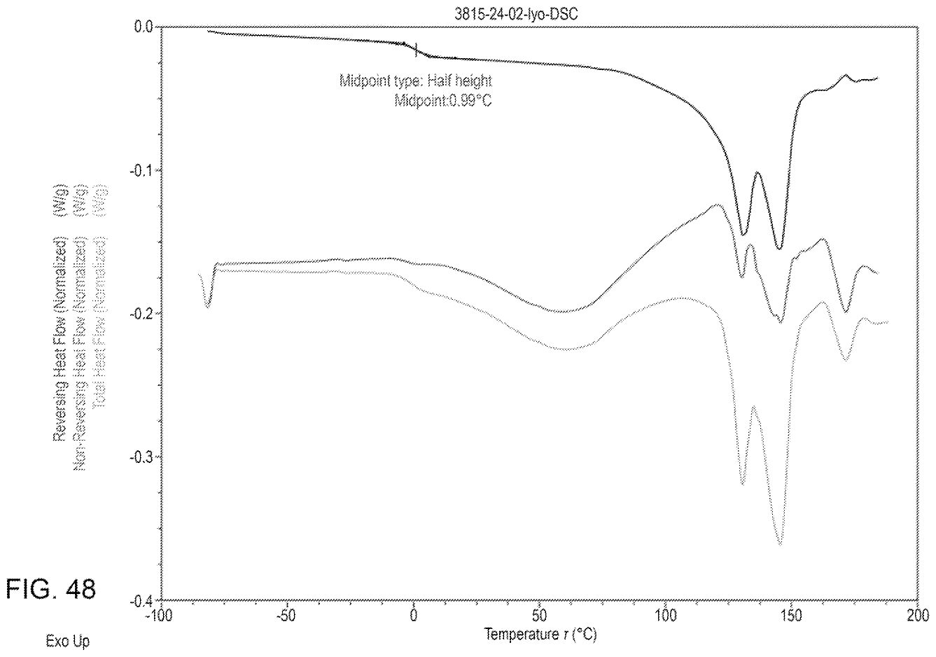

Demonstrates that the HBr at 50% loading in mannitol/trehalose does not produce an amorphous lyophilized product. FIG. 47 shows an XRPD of the lyophilized dispersion of Example 23. FIG. 48 shows a DSC thermogram for the lyophilized dispersion of Example 23.

### Example 24: Stable Amorphous Pharmaceutical Compositions or Formulations of 5-MeO-DMT HBr and HCl

The pharmaceutical compositions or formulations of 5-MeO-DMT HBr and HCl as described in Examples 16 and 17 were stored for 1 month at (i) 25 °C/60% RH or (ii) 2-8 °C. The pharmaceutical compositions or formulations comprised 50% by weight of either 5-MeO-DMT HBr or HCl, a 3:1 ratio of HPMC 606:Metolose 60SH 50 and 3% sorbitol.

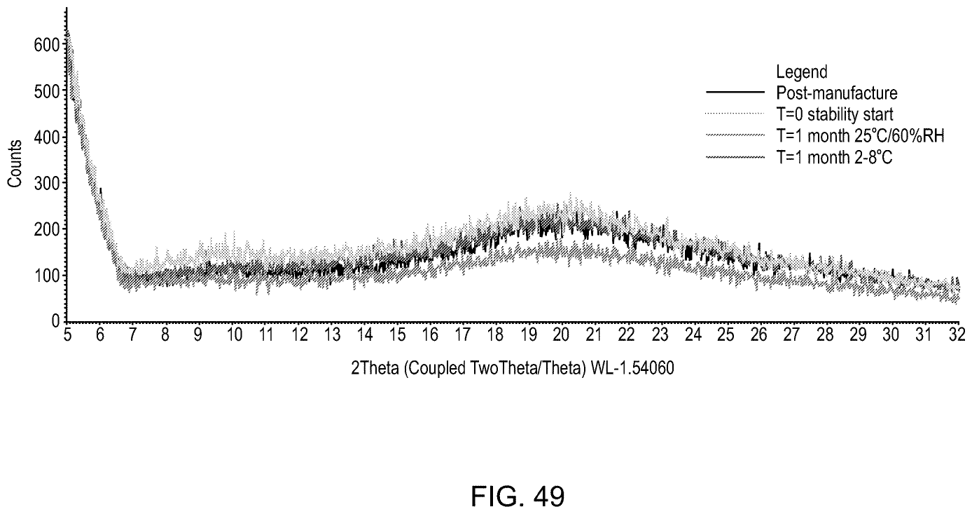

A pharmaceutical composition/formulation of 5-MeO-DMT HBr stored for 1 month at (i) 25 °C/60% RH or (ii) 2-8 °C remained amorphous. FIG. 49 shows an XRPD diffractogram for the HBr pharmaceutical composition/formulation of Example 16 at T=0, T=1 month post storage at 25 °C/60% RH and T=1 month post storage at 2-8 °C. The XRPD showed that the pharmaceutical composition/formulation remained amorphous. Analysis by TGA, mDSC, HPLC and scanning electron microscope (SEM) imaging further confirmed that the pharmaceutical composition/formulation remained amorphous.

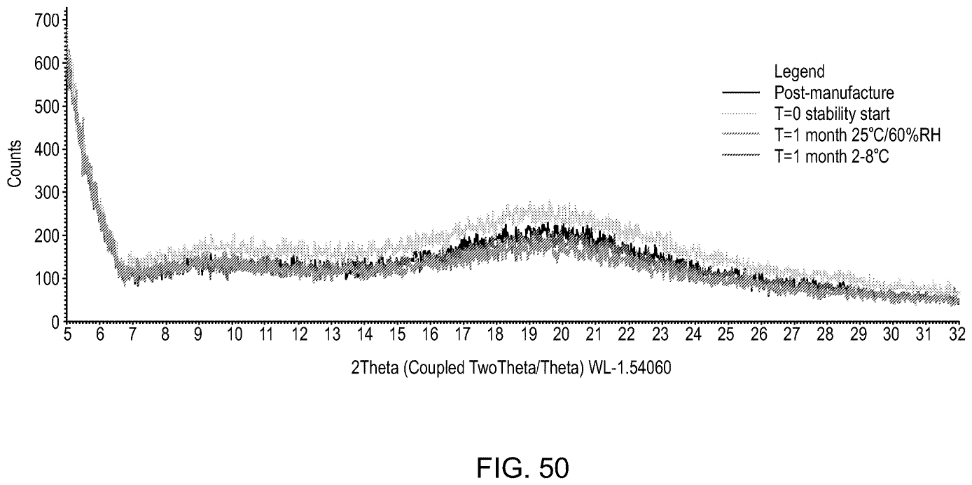

A pharmaceutical composition/formulation of 5-MeO-DMT HCl stored for 1 month at (i) 25 °C/60% RH or (ii) 2-8 °C remained amorphous. FIG. 50 shows an XRPD diffractogram for the HCl pharmaceutical composition/formulation of Example 17 at T=0, T=1 month post storage at 25 °C/60% RH and T=1 month post storage at 2-8 °C. The XRPD showed that the pharmaceutical composition/formulation remained amorphous. Analysis by TGA, mDSC, HPLC and scanning electron microscope (SEM) imaging further confirmed that the pharmaceutical composition/formulation remained amorphous. There is therefore provided, in an embodiment, a state-stable amorphous pharmaceutical composition/formulation of 5-MeO-DMT suitable for storage at, at least, 25 °C/60% RH for at least 1, 2, 3, 4, 5, 6, 7, 8, 9, 10, 11 or 12 months, wherein the pharmaceutical composition/formulation comprises 5-MeO-DMT HBr or 5-MeO-DMT HCl.

In an embodiment, there is provided a stable amorphous pharmaceutical composition/formulation of 5-MeO-DMT. In an embodiment, there is provided a state-stable amorphous pharmaceutical composition/formulation of 5-MeO-DMT. In an embodiment, there is provided a stable amorphous pharmaceutical composition/formulation of 5-MeO-DMT suitable for storage at, at least, 25 °C/60% RH for at least 1, 2, 3, 4, 5, 6, 7, 8, 9, 10, 11, 12 or 24 months. In an embodiment, there is provided an amorphous 5-MeO-DMT HBr formulation. In an embodiment, there is provided a method for producing a stable amorphous pharmaceutical composition/formulation of 5-MeO-DMT, as described herein. In an embodiment, there is provided a method for producing a state-stable pharmaceutical composition/formulation of 5-MeO-DMT, as described herein.

### Example 25: Method for the Determination of Dissolution Rate

The dissolution rate of SDD was determined using a method comprising the use of a UV-fibre optic-based dissolution apparatus (one such suitable device is the Rainbow® Dynamic Dissolution Monitor by Pion Inc) and simulated nasal fluid. The simulated nasal fluid comprises 7.45 g/L NaCl, 1.29 g/L KCl, 0.32 g/L CaCl₂)×2H₂O and deionised water.

The dissolution apparatus was set up with a 2 mm probe to measure the dissolution rate. 10 mg of the SDD (5 mg API) was transferred into 5 mL of simulated nasal fluid, heated to a constant temperature of 37 °C and stirred at 150 RPM using a crossed stirrer bar. Measurements were taken at 3 s intervals for 130 intervals followed by 60 measurements taken at 10 s intervals for a total time of 16 minutes and 30 s. Detection of dissolution is by UV absorbance.

In an embodiment, there is provided a method of determining the dissolution rate of a SDD. In an embodiment, the SDD is a 5-MeO-DMT SDD. In an embodiment, said 5-MeO-DMT SDD may be as described previously or subsequently herein.

In an embodiment, a 0.5, 1, 1.5, 2, 2.5, 3, 3.5, 4, 4.5 or 5 mm probe may be used. In an embodiment, 1, 2, 3, 4, 5, 6, 7, 8, 9, 10, 11, 12, 13, 14, 15, 16, 17, 18, 19 or 20 mg of the SDD may be used. In an embodiment, 1 to 100 mg of the SDD may be used. In an embodiment, 1-100 ml of simulated nasal fluid is used. In an embodiment, 1, 2, 3, 4, 5, 6, 7, 8, 9, 10, 11, 12, 13, 14, 15, 16, 17, 18, 19 or 20 mL of nasal fluid is used. In an embodiment, the constant temperature used is 37 °C+/−1, 2, 3, 4 or 5 °C. In an embodiment, the solution is stirred at 50, 60, 70, 80, 90, 100, 110, 120, 130, 140, 150, 160, 170, 180 190 or 200 RPM. In an embodiment, the solution is stirred at 50-200 RPM. In an embodiment, measurements are taken at 1, 2, 3, 4, 5, 6, 7, 8, 9 or 10 s intervals for 50, 60, 70, 80, 90, 100, 110, 120, 130, 140, 150, 160, 170, 180 190 or 200 intervals followed by 50, 60, 70, 80, 90, 100, 110, 120, 130, 140, 150, 160, 170, 180 190 or 200 measurements taken at 1, 2, 3, 4, 5, 6, 7, 8, 9 or 10 s intervals.

### Example 26: Method for the Determination of Crystalline Content

A method has been developed for the determination of the crystalline content of psychedelic pharmaceutical compositions or formulations. Differential scanning calorimetry (DSC) is a thermoanalytical technique in which the difference in the amount of heat required to increase the temperature of a sample and reference is measured as a function of temperature. Both the sample and reference are maintained at nearly the same temperature throughout the experiment. Differential scanning calorimetry can be used to measure a number of characteristic properties of a sample. Using this technique it is possible to observe fusion and crystallisation events as well as glass transition temperatures Tg.

The inventors have discovered that a heating rate of 10 °C per minute is not optimised for the determination of crystalline content in psychedelic pharmaceutical compositions or formulations. The enthalpy obtained from the melt of crystalline 5-MeO-DMT in a pharmaceutical composition/formulation was reduced with increased heating rate indicating that a DSC heating rate of 10 °C per minute is not suitable.

Surprisingly, heating rates of above 10 °C per minute is required in order to evaluate the crystalline content of psychedelic pharmaceutical compositions or formulations. The optimal heating rate has been discovered to be between 10° and 200 °C per minute, between 11° and 190 °C per minute, between 12° and 180 °C per minute, between 13° and 170 °C per minute or between 14° and 160 °C per minute. In an embodiment, the optimal heating rate for the determination of crystalline content of a psychedelic pharmaceutical composition/formulation by DSC is 150 °C per minute. In an embodiment, there is provided a method of determining the crystalline content of a 5-MeO-DMT pharmaceutical composition/formulation by DSC is 150 °C per minute. In an embodiment, there is provided a method of determining the crystalline content of a spray dried 5-MeO-DMT pharmaceutical composition/formulation by DSC is 150 °C per minute.

### Example 27: Further Stability Testing of Amorphous Pharmaceutical Compositions or Formulations of 5-MeO-DMT HBr and HCl

The pharmaceutical compositions or formulations of 5-MeO-DMT HBr and HCl as described in Examples 16 and 17 were stored for 2 months at (i) 25 °C/60% RH or (ii) 2-8 °C. The pharmaceutical compositions or formulations comprised 50% by weight of either 5-MeO-DMT HBr or HCl, a 3:1 ratio of HPMC 606:Metolose 60SH 50 and 3% sorbitol.

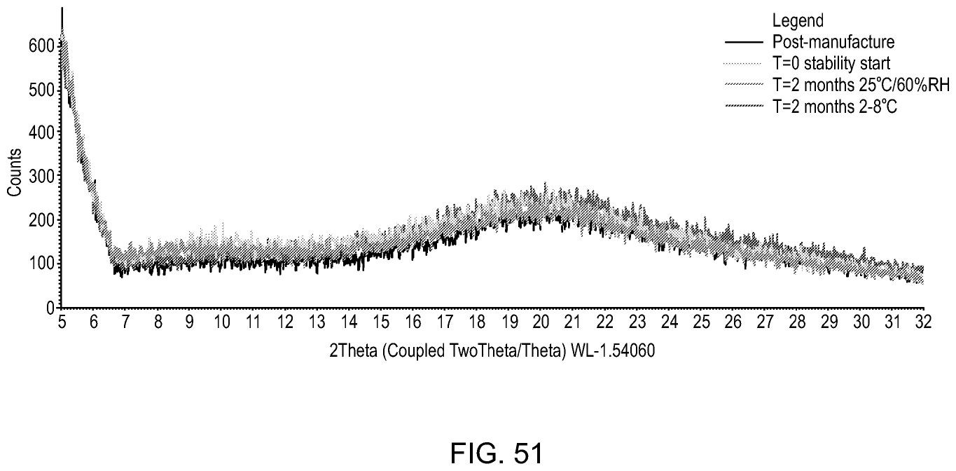

A pharmaceutical composition/formulation of 5-MeO-DMT HBr stored for 2 months at (i) 25 °C/60% RH or (ii) 2-8 °C remained amorphous. FIG. 51 shows an XRPD diffractogram for the HBr pharmaceutical composition/formulation of Example 16 at T=0, T=2 months post storage at 25 °C/60% RH and T=2 months post storage at 2-8 °C. The XRPD showed that the pharmaceutical composition/formulation remained amorphous. Analysis by TGA, mDSC, HPLC and scanning electron microscope (SEM) imaging further confirmed that the pharmaceutical composition/formulation remained amorphous.

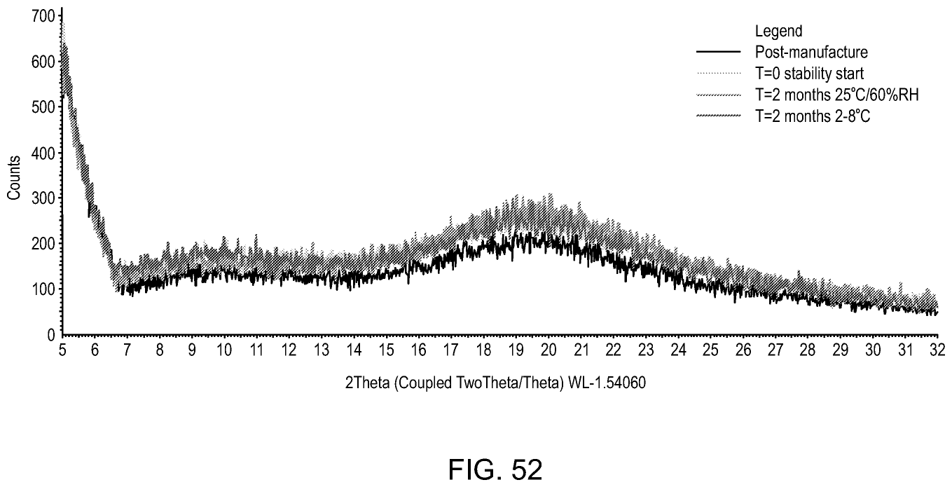

A pharmaceutical composition/formulation of 5-MeO-DMT HCl stored for 2 months at (i) 25 °C/60% RH or (ii) 2-8 °C remained amorphous. FIG. 52 shows an XRPD diffractogram for the HCl pharmaceutical composition/formulation of Example 17 at T=0, T=2 months post storage at 25 °C/60% RH and T=2 months post storage at 2-8 °C. The XRPD showed that the pharmaceutical composition/formulation remained amorphous. Analysis by TGA, mDSC, HPLC and scanning electron microscope (SEM) imaging further confirmed that the pharmaceutical composition/formulation remained amorphous.

At T=3 months and T=6 months, the above pharmaceutical compositions/formulations remained amorphous.

### Example 28: Nasal Deposition Profile Analysis

The nasal cavity is recognised as a promising systemic drug delivery route due to the highly vascularised capillary bed within the nasal mucosa. There is therefore a need for pharmaceutical compositions or formulations or pharmaceutical compositions or formulations as described herein with an optimised particle size distribution which show turbinate deposition. There is also a need for delivery devices which can selectively deliver a pharmaceutical composition/formulation as described herein to the nasal turbinates.

### Materials

5-methoxy-N,N-dimethyltryptamine (5-MeO-DMT) Benzoate (as described in Example 15) was provided by Beckley Psytech, Hydroxypropyl methylcellulose (HPMC) ((Pharmacoat 606-substitution 2910, viscosity 6 cP) ShinEtsu Chemical, Japan), HPLC grade 99% ethanol, HPLC grade 99% methanol, HPLC grade water, Glycerol and Brij-35 (Fisher Scientific, United Kingdom). Ultrapure water 18.2 MΩ (Veolia Elga LabWater System, in House) Active Devices (UDSp, Aptar Pharma, France).

### Preparation of 5-MeO-DMT Spray Dried Dispersion (5-MeO-DMT SDD)

Feed solution was prepared at 50% w/w 5-MeO-DMT Benzoate loading (32.1% 5-MeO-DMT). Both polymers were dissolved in water under ambient stirring overnight. D-sorbitol and 5-MeO-DMT Benzoate were added to solution and dissolved under ambient stirring, producing a clear, lightly straw-coloured solution. Feed solution was spray dried using the ProCepT 4M8-Trix Spray Dryer fitted with a 25 kHz Ultrasonic nozzle (ProCepT, Belgium), according to the spray drying parameters outlined in the Table below. pharmaceutical composition/formulation was filled and assembled into UDSp devices at 37.4±1.9 mg fill weight, under reduced humidity, when required for analysis.

Target Spray Drying Parameters for 5-MeO-DMT Feed Solution

| Inlet | Outlet | Nozzle | Cooling | Drying | Liquid |
|---|---|---|---|---|---|
| temperature | temperature | air | air | airflow | feed rate |
| (°C) | (°C) | (bar) | (bar) | (m³/min) | (g/min) |
| 105 | 80-85 | 2.00 | 0.5 | 0.70 | 2.0 |

### Particle Size Analysis by Laser Diffraction

Particle Size Distribution (PSD) was determined using a Sympatec HELOS H4459 particle size analyser equipped with an R5 lens (Sympatec GmbH, Germany) in triplicate. Bulk powder was analysed using the RODOS dry powder dispersion unit at 3 bar dispersal pressure and powder from active devices (ExDevice) were manually actuated into the laser diffractor with the tip of the device positioned 3 cm from the mid-point of the laser.

### Methodology—Summary

The Alberta Idealised Nasal Inlet (AINI) and Stage 1 collection cup of the Next Generation Impactor (NGI) was coated with a solution containing 12 g Brij-35, 20 g glycerol and 80 mL ethanol. Once dried, AINI and NGI were assembled with the addition of a pre-separator with 15 mL 50:50 (v/v) methanol:water diluent in the reservoir. The UDSp loaded with 37.4 mg pharmaceutical composition/formulation was positioned at either 30, 45 or 60° to the horizontal and inserted 1 cm into the nasal orifice of the AINI. A 7.5 L/min airflow was applied for 15 seconds upon actuation of the UDSp, delivering 1.875 L of air. After actuation, the configuration was disassembled and 15 mL diluent used to dissolve material on the UDSp's exterior, deposited in the AINI and the NGI collection cup, with the addition of a secondary dilution. Analysis was performed in triplicate and analysed by HPLC, with Two-way ANOVA statistical analysis.

### Methodology—Detailed

Nasal deposition was measured using the Alberta Idealised Nasal Inlet (AINI) with the Copley Next Generation Impactor (NGI) from an Aptar Unidose Powder Nasal spray system (UDSp)

### A) Coating of AINI and NGI Collection Cups

12 g Brij-35, 20 g glycerol and 80 mL ethanol were mixed until dissolved to form a coating solution. Bottom of the AINI was sealed and a 20 mL coating solution was added through the vestibule while the AINI was inverted. The AINI was slowly rotated horizontally 360° clockwise and anticlockwise then rotated vertically 360° clockwise and anticlockwise. Excess coating solution was drained and the AINI was placed on its left side, back and right side for 15 minutes each. AINI was positioned upright for 30 minutes to allow any further excess coating solution to drain and for the coat to dry.

2 mL of coating solution was pipetted onto the NGI Stage 1 collection cup and rocked for 5 minutes using the NGI rocker in order to coat. Excess solution was drained and the cup was allowed to dry.

### B) NGI Assembly

NGI was assembled with the coated Stage 1 collection cup and uncoated collection cups for states 2-7 and micro-orifice collector. The pre-separator and throat piece were attached and a leak test was performed using the critical flow controller and high-capacity pump. The flowmeter was attached to the throat piece and the flowrate set to 7.5 L/min.

The throat piece was removed and 15 mL 50:50% v/v HPLC grade water:HPLC grade methanol (diluent) was added to the pre-separator insert cup. The AINI was then installed on the pre-separator.

### C) Actuation

The UDSp containing 5-MeO-DMT was weighed to obtain a pre-actuation mass. The UDSp was then clamped into position such that the tip of the UDSp was inserted 1 cm into the vestibule of the AINI. The angle of insertion was set using an electronic protractor. The UDSp was actuated using 7.5 L/min flow rate for 15 seconds.

### D) HPLC Sample Collection

UDSp was then removed and weighed to obtain a post-actuation mass. The exterior of the UDSp was washed with 15 mL diluent in a glass dish and the washings were collected for HPLC analysis.

AINI was disassembled and each component was thoroughly washed in separate glass dishes with 15 mL diluent. These washings were then collected for HPLC analysis.

The pre-separator was removed from the NGI, the top and bottom were then covered and the pre-separator was inverted to wash the interior with the previously added 15 mL diluent. These washings were then collected for HPLC analysis.

15 mL diluent was added to the Stage 1 collection cup and the cup was rocked for 10 minutes using the NGI rocker to wash. These washings were then collected for HPLC analysis.

### E) HPLC

HPLC was carried out on the samples to quantify 5-MeO-DMT content. Where necessary samples were diluted to stay within the linearity of the quantification method.

In an embodiment, there is provided the use of the above described method, or similar, in a method of nasal deposition analysis of one or more of the pharmaceutical compositions or formulations or pharmaceutical compositions or formulations as described herein. In an embodiment, there is provided the use of the above described method, or similar, in a method of nasal deposition analysis of one or more of the 5-MeO-DMT pharmaceutical compositions or formulations or pharmaceutical compositions or formulations as described herein. In an embodiment, there is provided the use of the above described method, or similar, in a method of nasal deposition analysis of one or more psychedelic dry powder pharmaceutical compositions or formulations or pharmaceutical compositions or formulations. In an embodiment, there is provided the use of the above described method, or similar, in a method of nasal deposition analysis of a psychedelic dry powder formulation. In an embodiment, there is provided the use of the above described coating, or similar, in a method of psychedelic pharmaceutical composition/formulation nasal deposition analysis. In an embodiment, there is provided the use of the above described coating, or similar, in a method of nasal deposition analysis of a psychedelic dry powder formulation. In an embodiment, there is provided the use of the above described coating, or similar, in a method of nasal deposition analysis of one or more psychedelic dry powder pharmaceutical compositions or formulations or pharmaceutical compositions or formulations.

### Results

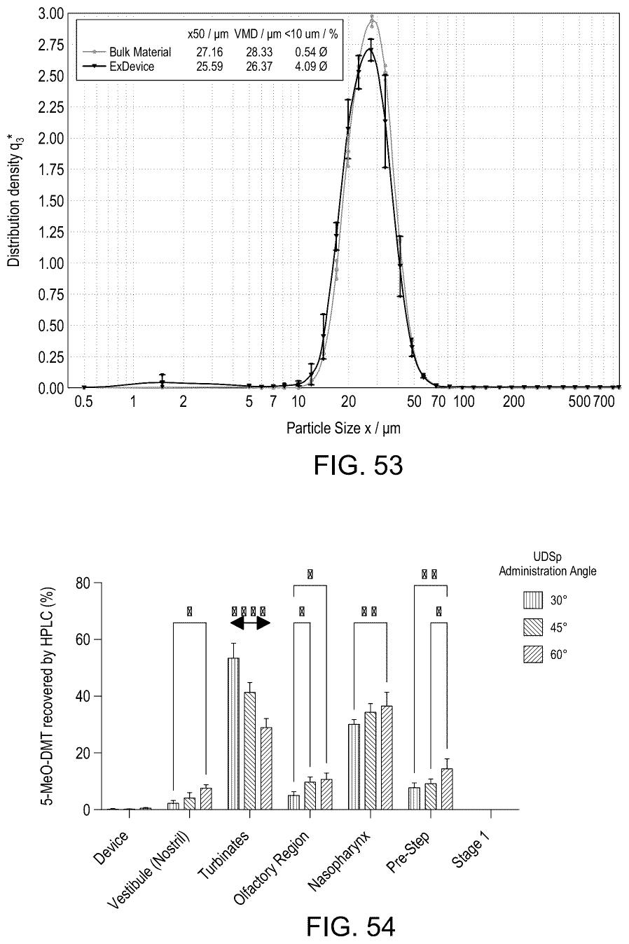

Pharmacopoeia guidelines state that a nasal powder should demonstrate that deposition of the products is localised within the nasal cavity, and the current method requires that most of the particles are larger than 10 μm as determined by laser diffraction. Analysis was performed on the 5-MeO-DMT SDD with the Sympatec, and the particle size distribution shown in FIG. 53. Using the ultrasonic nozzle an optimised nasal powder was achieved in which the mean particle diameter was close to the diameter recommended while maintaining minimal particles below 10 μm, passing acceptance criteria requested by EMA.

The AINI was used to assess the deposition profile of the 5-MeO-DMT SDD pharmaceutical composition/formulation with the method outlined above. The AINI consists of four components for the nasal cavity—the vestibule (nostril), turbinates, olfactory and nasopharynx—which is assembled and attached to a pre-separator. The pre-separator is incorporated to capture any deposition that would falsely land on Stage 1 due to particle bounce in the internal surfaces of the AINI. Minimal deposition was seen in the vestibule compared to commercially available nasal sprays. Minimal deposition was also seen in the lung analogue. The majority of the pharmaceutical composition/formulation was found in the turbinates and the olfactory region showed deposition from 5-11%—which is advantageous as it is theorised that a minimum of 0.01-1% of the oral dose is effective for nose-to-brain absorption. The results of the AINI can be seen in FIG. 54.

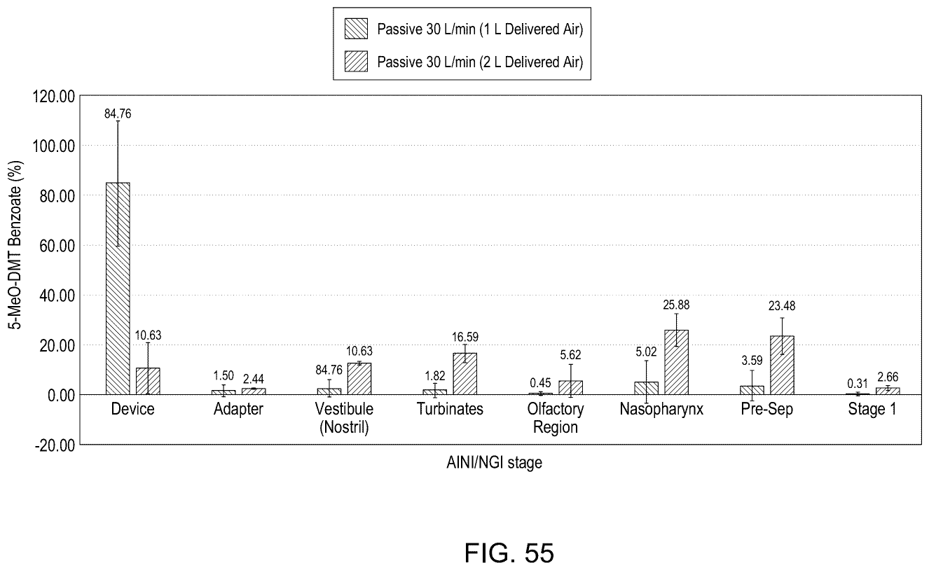

There is therefore provided an advantageous method for the delivery of a 5-MeO-DMT SDD. The same 5-MeO-DMT SDD (as per Example 15) was filled into the passive nasal delivery device at a loading suitable to deliver 12 mg 5MeO DMT free base equivalent. The passive device was positioned into an adapter and drawn through the AINI/NGI with a flow rate of 30 L/min to deliver either 1 L or 2 L of air respectively. The nasal deposition profile produced can be seen in FIG. 55. It can be readily seen that very little drug product was deposited in the desired locations of the turbinates and olfactory region.

There is therefore provided an advantageous method for the delivery of a 5-MeO-DMT SDD wherein said SDD is delivered by an active nasal delivery device. In an embodiment, there is provided the use of a pharmaceutical composition/formulation as described herein in a method of treating a patient in need thereof, wherein the pharmaceutical composition/formulation is administered intranasally via an active delivery nasal device, as described herein, and wherein more than 30%, 40%, 50%, 60%, 70%, 80% or 90% of the pharmaceutical composition/formulation is deposited to the turbinates and/or olfactory region of the nasal cavity.

In an embodiment, the method of treating a patient in need thereof is a method of treating one or more of the conditions or diseases described herein. In an embodiment, there is provided the use of a pharmaceutical composition/formulation as described herein in a method of treating a patient in need thereof, wherein the pharmaceutical composition/formulation is administered intranasally via an active delivery nasal device and wherein less than 30%, 25%, 20%, 15%, 10%, 5%, 4%, 3%, 2% or 1% is deposited in the lungs.

In an embodiment, there is provided a nasal delivery device for delivering a pharmaceutical composition/formulation as described herein to an olfactory region of a nasal cavity, the device comprising a pharmaceutical composition/formulation as described herein. In an embodiment, the device is an active nasal delivery device wherein a plunger style actuator, or similar, is depressed to administer a dose. In an embodiment, the nasal delivery device is not a breath actuated delivery device. In an embodiment, the device comprises a dose volume up to 140 mm³. In an embodiment, the nasal delivery device may be as described in any one of WO21005308; WO22123128; WO22171969; and WO22208014 (the contents of which are incorporated by reference).

In an embodiment, there is provided a dispenser device, optionally for dispensing a pharmaceutical composition/formulation as described herein, the dispenser device comprising: a pharmaceutical composition/formulation as described herein; a dispenser outlet (10); an air expeller (20) for generating a flow of air while the device is being actuated, said air expeller (20) including a piston (21) that slides in an air chamber (22) between a rest position and a dispensing position, said air chamber (22) including a cylindrical body (222) in which said piston (21) slides in airtight manner; and at least one reservoir (30) that contains a single dose of formulation, said reservoir (30) including an air inlet (31) that is connected to said air expeller (20), and a pharmaceutical composition/formulation outlet (32) that is connected to said dispenser outlet (10), said air inlet (31) including a pharmaceutical composition/formulation retainer member (40) for retaining the pharmaceutical composition/formulation in the reservoir (30) until the pharmaceutical composition/formulation is dispensed, and said pharmaceutical composition/formulation outlet (32) being closed by a closure element (50) that is force fitted in the pharmaceutical composition/formulation outlet (32) of the reservoir (30); said device further including a mechanical opening system (61, 62) that co-operates with said closure element (50) so as to expel it mechanically from its closed position while the device is being actuated, said mechanical opening system comprising a rod assembly (61, 62), a first rod portion (61) being part of said air expeller (20) and sliding in said air chamber (22) during actuation of the device, and a second rod portion (62) pushed by said first rod portion (61) during actuation of the device, said rod assembly (61, 62) cooperating at the end of the actuation stroke with said closure element (50) to expel it mechanically from its closed portion, said piston (21) of said air expeller (20), when in its rest position, co-operating in non-airtight manner with said air chamber (22), in such a manner that said air chamber (22) is in communication with the atmosphere in the rest position, said piston (21) including an inner lip (215) that slides in airtight manner on said cylindrical surface (614) during actuation of the device, and that co-operates in non-airtight manner with fluting (615) formed on said cylindrical surface (614) in the rest position to put the air chamber (22) in communication with the atmosphere in the rest position, said piston 21) co-operating in airtight manner with said cylindrical body (222) in any positions, and co-operating in non-airtight manner with said cylindrical surface (614) only in the rest position.

In an embodiment, there is provided a dispenser device, optionally for dispensing a pharmaceutical composition/formulation as described herein, the dispenser device comprising: a pharmaceutical composition/formulation as described herein; a dispenser outlet; an air expeller for generating a flow of air while the device is being actuated, said air expeller including a piston that slides in an air chamber between a rest position and a dispensing position, said air chamber including a cylindrical body in which said piston slides in airtight manner; and at least one reservoir that contains a single dose of formulation, said reservoir including an air inlet that is connected to said air expeller, and a pharmaceutical composition/formulation outlet that is connected to said dispenser outlet, said air inlet including a pharmaceutical composition/formulation retainer member for retaining the pharmaceutical composition/formulation in the reservoir until the pharmaceutical composition/formulation is dispensed, and said pharmaceutical composition/formulation outlet being closed by a closure element that is force fitted in the pharmaceutical composition/formulation outlet of the reservoir; said device further including a mechanical opening system that co-operates with said closure element so as to expel said closure element mechanically from a closed position while the device is being actuated, said piston of said air expeller, when in the rest position, co-operating in non-airtight manner with said air chamber, in such a manner that said air chamber is in communication with the atmosphere in the rest position, wherein said piston includes an inner lip configured to cooperate with a cylindrical surface of a cylindrical member extending inside the cylindrical body, said cylindrical surface including fluting that co-operates in non-airtight manner with said inner lip of the piston in the rest position.

A method of intranasally delivering a powder pharmaceutical composition/formulation comprising a psychedelic and one or more pharmaceutically acceptable carriers or excipients, to a patient, wherein 30% or more, 40% or more, 50% or more, 60% or more, 70% or more, 80% or more, 90% or more, 91% or more, 92% or more, 93% or more, 94% or more, 95% or more, 96% or more, 97% or more, 98% or more or 99% or more of the pharmaceutical composition/formulation reaches the turbinates and olfactory region, wherein the psychedelic is 5-MeO-DMT, or a pharmaceutically acceptable salt thereof, and the pharmaceutical composition/formulation is delivered via a nasal powder dispenser device which may comprise one or more of: a nasal dispenser head for inserting into a patient's nostril, the nasal dispenser head including a dispenser orifice; and an air expeller that, during actuation of the nasal powder dispenser device, generates a flow of compressed air so as to dispense a dose of the powder pharmaceutical composition/formulation into the nostril through the dispenser orifice.

In an embodiment, there is provided an intranasal delivery system, optionally comprising an active nasal delivery device as described herein, comprising: a dry powder pharmaceutical composition/formulation comprising a plurality of powder particles of 5-MeO-DMT, or a pharmaceutically acceptable salt thereof; and an active nasal delivery device, optionally as described herein, configured to deliver the particles to the turbinates and olfactory region of the nasal cavity of a subject at a single actuation; wherein the system is operably configured to emit a powder plume comprising 5-MeO-DMT, or a pharmaceutically acceptable salt thereof, having one or more of: a plume geometry of: angle: 20 to 45 degrees; width: 25 to 55 mm;
a spray pattern of:

| Distance 40 mm: | Distance 70 mm: |
|---|---|
| Dmin(mm): 15 to 25 | Dmin(mm): 15 to 40 |
| Dmax(mm): 20 to 55 | Dmax(mm): 35 to 60 |
| Area (mm²): 390 to 900 | Area (mm²): 800-1600 |
| Area %: 2 to 15 | Area %: 4 to 20 |

a particle size distribution (at 40 mm) of:
   D10=13 to 17, D50=35 to 60, D90=650 to 700, %<10 μm=<0.1, 1, 2, 3, 4, 5, 6, 7, 8, 9 or 10%; or
a particle size distribution (at 70 mm) of:
   D10=13 to 17, D50=24 to 30, D90=540 to 610, %<10 μm=<0.1, 1, 2, 3, 4, 5, 6, 7, 8, 9 or 10%; or
a particle size distribution of:
   D10=13 to 17, D50=22 to 27, D90=35 to 56, %<9 μm=<0.1-10%; or
% particles of equal to or less than 11.7 μm size of:
   0.5 to 5%.

In an embodiment, there is provided an intranasal delivery system, optionally comprising an active nasal delivery device as described herein, comprising: a dry powder pharmaceutical composition/formulation comprising a plurality of powder particles of 5-MeO-DMT, or a pharmaceutically acceptable salt thereof; and an active nasal delivery device, optionally as described herein, configured to deliver the particles to the turbinates and olfactory region of the nasal cavity of a subject at a single actuation; wherein the system is operably configured to emit a powder plume comprising 5-MeO-DMT, or a pharmaceutically acceptable salt thereof, having one or more of: a plume geometry of: angle: 20 to 45 degrees; width: 25 to 55 mm; a spray pattern of:

| Distance 40 mm: | Distance 70 mm: |
|---|---|
| Dmin(mm): 15 to 25 | Dmin(mm): 25 to 40 |
| Dmax(mm): 35 to 55 | Dmax(mm): 45 to 60 |
| Area (mm²): 600 to 850 | Area (mm²): 1100 to 1700 |
| Area %: 1 to 5 | Area %: 4 to 8 |

a particle size distribution (at 40 mm) of: D10=13 to 17, D50=35 to 60, D90=650 to 700, %<10 μm=<0.1, 1, 2, 3, 4, 5, 6, 7, 8, 9 or 10%; or
a particle size distribution (at 70 mm) of: D10=13 to 17, D50=24 to 30, D90=540 to 610, %<10 μm=<0.1, 1, 2, 3, 4, 5, 6, 7, 8, 9 or 10%; or
% particles of equal to or less than 11.7 μm size of: 0.5 to 5%.

In an embodiment, there is provided an intranasal delivery system, optionally comprising an active nasal delivery device as described herein, comprising: a dry powder pharmaceutical composition/formulation comprising a plurality of powder particles of 5-MeO-DMT, or a pharmaceutically acceptable salt thereof; and an active nasal delivery device, optionally as described herein, configured to deliver the particles to the turbinates and olfactory region of the nasal cavity of a subject at a single actuation; wherein the system is operably configured to emit a powder plume comprising 5-MeO-DMT, or a pharmaceutically acceptable salt thereof, having one or more of: a plume geometry of: angle: 20 to 35 degrees; width: 25 to 45 mm; a spray pattern of:

| Distance 40 mm: | Distance 70 mm: |
|---|---|
| Dmin(mm): 15 to 25 | Dmin(mm): 15 to 40 |
| Dmax(mm): 20 to 35 | Dmax(mm): 35 to 50 |
| Area (mm²): 390 to 460 | Area (mm²): 800-1200 |
| Area %: 5 to 15 | Area %: 7 to 20 |

a particle size distribution of: D10=13 to 17, D50=22 to 27, D90=35 to 56, %<9 μm=<0.1-10%; or % particles of equal to or less than 11.7 μm size of: 0.5 to 5%.

In an embodiment, there is provided an intranasal delivery system, optionally comprising an active nasal delivery device as described herein, comprising: a dry powder pharmaceutical composition/formulation comprising a plurality of powder particles of 5-MeO-DMT, or a pharmaceutically acceptable salt thereof; and an active nasal delivery device, optionally as described herein, configured to deliver the particles to the turbinates and olfactory region of the nasal cavity of a subject at a single actuation; wherein the system is operably configured to emit a powder plume comprising 5-MeO-DMT, or a pharmaceutically acceptable salt thereof, having one or more of: a plume geometry of: angle: 27 or 40 degrees; width: 33 or 50 mm; a spray pattern of:

| Distance 40 mm: | Distance 70 mm: |
|---|---|
| Dmin(mm): 21 or 22 | Dmin(mm): 33 or 34 |
| Dmax(mm): 41 or 47 | Dmax(mm): 52 or 54 |
| Area (mm²): 640 or 827 | Area (mm²): 1230 or 1545 |
| Area %: 3 or 4 | Area %: 5 or 7 |

a particle size distribution (at 40 mm) of: D10=15 or 16, D50=38 or 54, D90=684 or 685, %<10 μm=<0.1, 1, 2, 3, 4, 5, 6, 7, 8, 9 or 10%; or a particle size distribution (at 70 mm) of: D10=15 or 16, D50=27 or 28, D90=558 or 596, %<10 μm=<0.1, 1, 2, 3, 4, 5, 6, 7, 8, 9 or 10%; or a particle size distribution of: D10=13 to 17, D50=22 to 27, D90=35 to 56, %<9 μm=<0.1-10%; or % particles of equal to or less than 11.7 μm size of: 0.5 to 5%.

In an embodiment, the powder plume of 5-MeO-DMT, or a pharmaceutically acceptable salt thereof, has a plume geometry of: angle: 22 to 35 degrees; width: 27 to 55 mm; or plume geometry of: angle: 22 to 33 degrees; width: 27 to 42 mm; or a plume geometry of: angle: 25 to 30 degrees; width: 29 to 39 mm; or a plume geometry of: angle: 26 to 28 degrees; width: 32 to 35 mm; or a plume geometry of: angle: 27.5 degrees; width: 34.33 mm; or a plume geometry of: angle: 24.4 degrees; width: 30.30 mm; or a plume geometry of: angle: 24.8 degrees; width: 30.76 mm; or a plume geometry of: angle: 27.4 degrees; width: 34.13 mm; or a plume geometry of: angle: 30.5 degrees; width: 38.29 mm; or a plume geometry of: angle: 39.2 degrees; width: 50.35 mm; or a plume geometry of: angle: 33.43 degrees; width: 52.25 mm; or a plume geometry of: angle: 28 degrees; width: 34 mm; or a plume geometry of: angle: 24 degrees; width: 30 mm; or a plume geometry of: angle: 425 to 43525 degrees; width: 31 mm; or a plume geometry of: angle: 27 degrees; width: 34 mm; a plume geometry of: angle: 31 degrees; width: 38 mm or a plume geometry of: angle: 20, 21, 22, 23, 24, 25, 26, 27, 28, 29, 30, 31, 32, 33, 34, 35, 36, 37, 38, 39, 40, 41, 42, 43, 44, 45 or 46 degrees; width: 26, 27, 28, 29, 30, 31, 32, 33, 34, 35, 36, 37, 38, 39, 40, 41, 42, 43, 44, 45, 46, 47, 48, 49, 50, 51, 52, 53, 54, 55 or 56 mm.

In an embodiment, the powder plume of 5-MeO-DMT, or a pharmaceutically acceptable salt thereof, has a spray pattern of:

| Distance 40 mm: | Distance 70 mm: |
|---|---|
| Dmin(mm): 18 to 22 | Dmin(mm): 20 to 30 |
| Dmax(mm): 25 to 30 | Dmax(mm): 40 to 45 |
| Area (mm²): 400 to 450 | Area (mm²): 900-1100 |
| Area %: 7 to 13 | Area %: 9 to 15 |

or

| Distance 40 mm: | Distance 70 mm: |
|---|---|
| Dmin(mm): 19 to 21 | Dmin(mm): 24 to 28 |
| Dmax(mm): 27 to 29 | Dmax(mm): 42 to 44 |
| Area (mm²): 400 to 450 | Area (mm²): 950-1050 |
| Area %: 8 to 11 | Area %: 12 to 14 |

In an embodiment, the powder plume of 5-MeO-DMT, or a pharmaceutically acceptable salt thereof, has a spray pattern of:

| Distance 40mm: | Distance 70 mm: |
|---|---|
| Dmin(mm): 20.43 or 20.17 or | Dmin(mm): 20.84 or 29.23 or |
| 17.79 or 20.09 | 30.67 or 31.10 |
| Dmax(mm): 27.59 or 24.32 or | Dmax(mm): 43.69 or 38.83 or |
| 29.68 or 27.46 | 42.82 or 43.31 |
| Area (mm²): 431.0 or 423.7 | Area (mm²): 1048.5 or 919.4 |
| or 405.6 or 449.0 | or 1038.8 or 1029.2 |
| Area %: 8.1 or 7.8 or | Area %: 10.3 or 10.1 or |
| 7.5 or 8.3 | 9.0 or 10.1 |

or

| Distance 40 mm: | Distance 70 mm: |
|---|---|
| Dmin(mm): 21.45 or 22.10 | Dmin(mm): 33.43 or 33.43 |
| Dmax(mm): 40.66 or 47.23 | Dmax(mm): 52.25 or 53.50 |
| Area (mm²): 639.6 or 827.1 | Area (mm²): 1229.6 or 1544.9 |
| Area %: 2.7 or 3.5 | Area %: 5.3 or 6.6 |

or

| Distance 40 mm: | Distance 70 mm: |
|---|---|
| Dmin(mm): 20 or 18 | Dmin(mm): 21 or 29 or 31 |
| Dmax(mm): 28 or 24 or 27 | Dmax(mm): 44 or 39 or 43 |
| Area (mm²): 431 or 424 | Area (mm²): 1049 or 919 |
| or 406 or 449 | or 1039 or 1029 |
| Area %: 2.7 or 3.5 | Area %: 9 or 10 |

or

| Distance 40 mm: | Distance 70 mm: |
|---|---|
| Dmin(mm): 18 to 20 | Dmin (mm): 20 to 32 |
| Dmax(mm): 24 to 28 | Dmax(mm): 39 to 45 |
| Area (mm²): 405 to 450 | Area (mm²): 915 to 1050 |
| Area %: 7 to 9 | Area %: 9 to 11 |

In an embodiment, the particle size distribution of the powder plume of 5-MeO-DMT, or a pharmaceutically acceptable salt thereof, is: D10=15.54 or 15.05 or 15.89 or 15.31, D50=26.8 or 28.21 or 38.27 or 53.92, D90=558.4 or 595.9 or 683.8 or 385.3, %<10 μm=5.45 or 3.01 or 3.90 or 3.79; or D10=10.4 or 10.7, D50=21.0 or 22.8, D90=38.4 or 14.9, %<10 μm=8.65% or 8.35%; or D10=13, 14, 15, 16 or 17, D50=22, 23, 24, 25, 26 or 27, D90=35, 36, 37, 38, 39, 40, 41, 42, 43, 44, 45, 46, 47, 48, 49, 50, 51, 52, 53, 54, 55 or 56, %<9 μm=<0.1, 1, 2, 3, 4, 5, 6, 7, 8, 9 or 10%.

In an embodiment, the % particles of equal to or less than 11.7 μm size of the powder plume of 5-MeO-DMT, or a pharmaceutically acceptable salt thereof, is: 0.5 to 5%, 0.6 to 4%, 0.7 to 3%, 0.8 to 2%, 0.9 to 1%. In an embodiment, the active nasal delivery has an actuation force of between 30 and 60N. In an embodiment, the actuation force is between 40 and 50N. In an embodiment, the actuation force is 41, 42, 43, 44, 45, 46, 47, 48 or 49N. In an embodiment, the actuation force is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80N. In an embodiment, the actuation force is 36N. In an embodiment, the actuation force is 37N. In an embodiment, the actuation force is 38N. In an embodiment, the actuation force is 39N. In an embodiment, the actuation force is 36N.

In an embodiment, the dry powder pharmaceutical composition/formulation comprising a plurality of powder particles of 5-MeO-DMT, or a pharmaceutically acceptable salt thereof, comprises a crystalline form of 5-MeO-DMT, or a pharmaceutically acceptable salt thereof, as described herein. In an embodiment, the dry powder pharmaceutical composition/formulation comprising a plurality of powder particles of 5-MeO-DMT, or a pharmaceutically acceptable salt thereof, has a moisture content of <5%, <4%, <3%, <2%, or <1%. In an embodiment, the moisture content is <2%, <1.9%, <1.8%, <1.7%, <1.6%, <1.5%, <1.4%, <1.3%, <1.2% or <1.1%. In an embodiment, the dry powder pharmaceutical composition/formulation comprising a plurality of powder particles of 5-MeO-DMT, or a pharmaceutically acceptable salt thereof, has <5%, <4%, <3%, <2%, <1%, <0.9%, <0.8%, <0.7%, <0.6%, <0.5%, <0.4%, <0.3%, <0.2%, <0.1%, <0.09%, <0.08%, <0.07%, <0.06%, <0.05%, <0.04%, <0.03%, <0.02% or <0.01% by weight of a hydroxyl impurity.

In an embodiment, the dry powder pharmaceutical composition/formulation comprising a plurality of powder particles of 5-MeO-DMT, or a pharmaceutically acceptable salt thereof, has <5%, <4%, <3%, <2%, <1%, <0.9%, <0.8%, <0.7%, <0.6%, <0.5%, <0.4%, <0.3%, <0.2%, <0.1%, <0.09%, <0.08%, <0.07%, <0.06%, <0.05%, <0.04%, <0.03%, <0.02% or <0.01% of by weight of any one impurity. In an embodiment, the dry powder pharmaceutical composition/formulation comprising a plurality of powder particles of 5-MeO-DMT, or a pharmaceutically acceptable salt thereof, has <5%, <4%, <3%, <2%, <1%, <0.9%, <0.8%, <0.7%, <0.6%, <0.5%, <0.4%, <0.3%, <0.2%, <0.1%, <0.09%, <0.08%, <0.07%, <0.06%, <0.05%, <0.04%, <0.03%, <0.02% or <0.01% of by weight of any impurity.

In an embodiment, the impurity profile is determined by RP-HPLC. In an embodiment, the % particles of equal to or less than 11.7 μm size of the powder plume is determined by Next Generation Impactor and HPLC. In an embodiment, the moisture content is determined by Karl Fisher coulometric titration. In an embodiment, the plume geometry is analysed using the Proveris SprayVIEW apparatus (or equivalent) in conjunction with the Proveris automated actuation device. In an embodiment, Analysis is performed at one distance (7.0 cm). In an embodiment, the settings are as follows: Orifice tip distance (cm): 7.0, Frame rate (Hz): 500, Number of images 250, Lens aperture 2.0, Camera position from horizontal (cm): 27.0, Camera height (cm): 8.0, Laser position (cm): 5.2, Laser depth (cm): 5.3, Laser height (cm): 13.2, Actuator position (cm): 7.0, Plume orientation: 0 deg, Palette: Gradient, Arm 1/Arm 2(%): 20-30%, Evacuation time (ms): 1000, Setting time (ms): 1000.

In an embodiment, the spray pattern is determined using the Proveris SprayVIEW apparatus (or equivalent) in conjunction with the Proveris automated actuation device. In an embodiment, analysis is performed at two distances (4.0 cm and 7.0 cm). In an embodiment, the settings are as above for the 7.0 cm distance and as follows for the 4.0 cm distance (where different from the settings used for 7.0 cm): Orifice tip distance (cm): 4.0, Camera position from horizontal (cm): 8.0 and Camera height (cm): 22.

In an embodiment, the particle size distribution is determined by laser diffraction using a Malvern Mastersizer (or equivalent). In an embodiment, the settings are as follows: Instrument: Malvern Mastersizer 3000 with Malvern software (or equivalent), Sampling handling Unit: Hydro MV dispersion unit, Material Refractive Index: 1.590, Absorption Refractive Index: 0.001, Dispersant Refractive Index: 1.391 (2,2,4-trimethylpentane), Obscuration Limits: 10-20%, Sonification time: Externally sonicated for 120 secs during sample preparation prior to addition to Hydro MV, Stirrer Speed: 3000 rpm, Measurement time: 30 secs, Background time: 30 secs, Dispersant: 2,2,4-trimethylpentane (RI=1.391) and Lecithin 0.05% w/w, degassed and equilibrated to ambient temperature.

In an embodiment, the aerodynamic particle size distribution (DISP) is determined by a method based on USP <601>, using the Proveris Sprayview and a Copley Next Generation Impactor (NGI) or equivalent, complying with USP/Ph.Eur. In an embodiment, standard solutions are prepared based on the label claim for the drug product (xmg per 100 ml diluent) where x=label claim. In an embodiment, the settings are as follows: Actuation acceleration: 5000 mm/s/s, Actuation velocity: 70 mm/s, Symmetric: Yes, Initial delay: 0 ms, Hold time: 100 ms, Final delay: 0 ms, Stroke length: 14 mm, One shot is fired into the NGI. Weigh the device prior to (W1) and after firing (W2) to calculate shot weight (W3). W1-W2=shot weight (W3), Add 5 ml of test solvent to each NGI cup then place on the NGI gentle rocker for 5 minutes. Quantitatively wash the expansion chamber, bungs, inlet cone, all cups and the Proveris collar with diluent into the correct flask size and make to volume. Assay is determined via HPLC.

In an embodiment, the particle size distribution (PSD) is determined by laser diffraction. In an embodiment, the analysis is performed using Sympatec instrumentation with R5 lens and a dispersal pressure of 3 bar. The intranasal delivery system/device is held in a clamp stand and positioned central with the extractor and so the tip of the device is 3 cm from the mid-point of the laser. After referencing, the device is manually/hand actuated so the powder passes through the laser beam, which takes a reading. Readings are performed with an R5 lens, in triplicate and then an average calculated.

### Example 29: Further 5-MeO-DMT Pharmaceutical Compositions or Formulations

A spray dried pharmaceutical composition/formulation of 50% 5-MeO-DMT benzoate with 34.5% HPMC 606, 3% isomalt and 12.5% methyl cellulose (MC) SM-100 grade was produced. The isomalt used was an agglomerated spherical isomalt (galenIQ 721) with the following properties: Solubility: 42 g/100 g solution at 20° C. water and Bulk density: 0.40 g/cm³.

SDD formulation

| | |
|---|---|
| Sample Reference | 3815-46-08 |
| Pharmacoat 606 | 34.5% |
| MC SM-100 | 12.5% |
| Isomalt | 3% |
| 5-MeO-DMT benzoate | 50% |

Spray Drying Parameters

| | |
|---|---|
| Instrument | Procept |
| Nozzle | Twin-fluid nozzle |
| Inlet Flow (m³/min) | 0.7 |
| Inlet Temperature (°C) | 130 |
| Cyclone Gas Pressure (bar) | 1.2-1.5 |
| Pump Speed (RPM) | 100 (~1.6-2 g per minute) |
| Feed Stock % solids | 10 |

Yield

| | |
|---|---|
| Yield (%) | 44.519% |

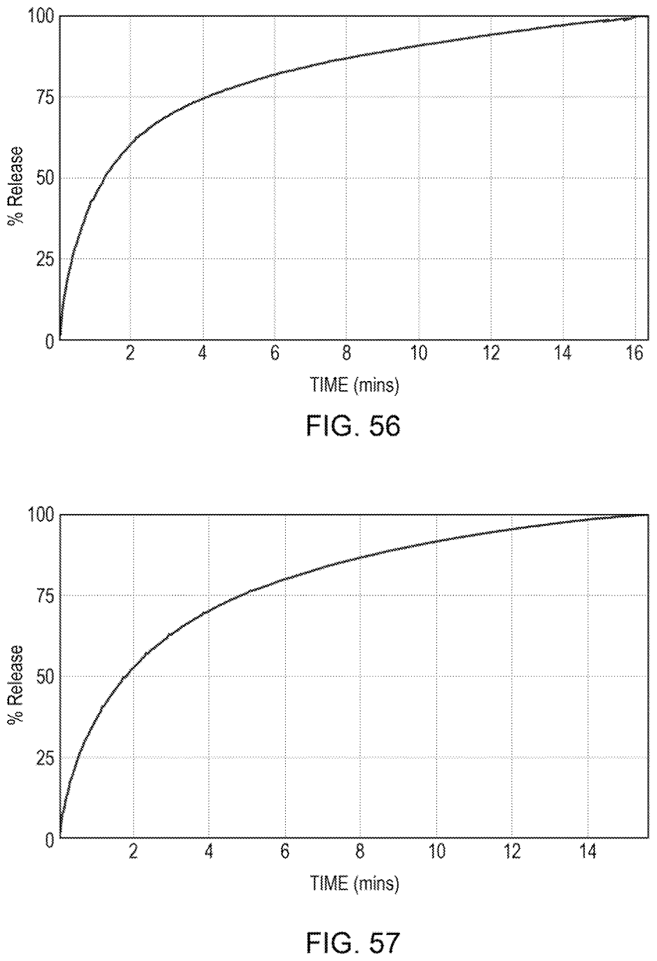

The dissolution profile, shown in FIG. 56, shows that for this SDD~80% release has occurred by ~6 minutes. In an embodiment, there is therefore provided a 5-MeO-DMT pharmaceutical composition/formulation comprising HPMC, isomalt and methyl cellulose. In an embodiment, there is therefore provided an extended release pharmaceutical composition/formulation of 5-MeO-DMT. A spray dried pharmaceutical composition/formulation of 50% 5-MeO-DMT benzoate with 3% sorbitol and 47% MC SM-25 was produced.

SDD formulation

| | |
|---|---|
| Sample Reference | 3815-46-09 |
| MC SM-25 | 47% |
| Sorbitol | 3% |
| 5-MeO-DMT benzoate | 50% |

Spray Drying Parameters

| | |
|---|---|
| Instrument | Procept |
| Nozzle | Twin-fluid nozzle |
| Inlet Flow (m³/min) | 0.7 |
| Inlet Temperature (°C.) | 130 |
| Cyclone Gas Pressure (bar) | 2 |
| Pump Speed (RPM) | 100 −> 400 to achieve ~0.8 |
| | g/min feed rate. |
| Feed Stock % solids | 10 |

Yield

| | |
|---|---|
| Yield (%) | 36.017% |

The dissolution profile, shown in FIG. 57, shows that for this SDD~80% release has occurred by ~6 minutes. In an embodiment, there is therefore provided a 5-MeO-DMT pharmaceutical composition/formulation comprising methyl cellulose and sorbitol. In an embodiment, there is therefore provided an extended release pharmaceutical composition/formulation of 5-MeO-DMT.

### Example 30: 5-MeO-DMT Buccal Pharmaceutical Compositions or Formulations

Buccal pharmaceutical compositions or formulations of 5-MeO-DMT have been developed and are characterised below:

| | (% w/w) | | | | | |
|---|---|---|---|---|---|---|
| Component | S01 | S12 | S26 | S27 | S28 | S29 |
| 5-MeO-DMT | 3.14 | 3.14 | 3.14 | 1.57 | 3.14 | 6.27 |
| benzoate | | | | | | |
| 5-MeO-DMT | 2.01 | 2.01 | 2.01 | 1.01 | 2.01 | 4.02 |
| (corrected | | | | | | |
| for salt) | | | | | | |
| Ethanol | 30.00 | 30.00 | 60.00 | 30.00 | 30.00 | 35.00 |
| Water | — | 0.99 | — | — | — | — |
| Propylene | 2.00 | 2.00 | 2.00 | 2.00 | 2.00 | 5.00 |
| glycol | | | | | | |
| Eudragit | — | 2.00 | — | — | — | — |
| RS100 | | | | | | |
| Eudragit | 2.00 | — | 2.00 | 2.00 | — | 2.00 |
| E100 | | | | | | |
| HFA 134a | 62.86 | 61.87 | 32.86 | 64.43 | 64.86 | 51.73 |
| Total | 100.00 | 100.00 | 100.00 | 100.00 | 100.00 | 100.00 |

The pharmaceutical compositions or formulations were evaluated in an in vitro nasal epithelium permeation experiment using ovine nasal epithelium. The mean cumulative amount of 5-MeO-DMT (μg/mL, % applied dose) delivered to the receptor solution at 1 h and peak flux (μg/min), following application of the 6 pharmaceutical compositions or formulations to reconstructed oral tissues is shown below:

| | Cumulative (μg/mL) | | | Peak Flux (ug/min) | | | % Dosed (cumulative) | | |
|---|---|---|---|---|---|---|---|---|---|
| Formulation | N | Mean | Std Dev | N | Mean | Std Dev | N | Mean | Std Dev |
| S01 | 4 | 33.95 | 8.69 | 4 | 1.61 | 0.41 | 4 | 24.89% | 6.37% |
| S12 | 5 | 51.51 | 11.38 | 5 | 1.60 | 0.64 | 5 | 86.57% | 19.13% |
| S26 | 5 | 24.63 | 3.04 | 5 | 1.10 | 0.41 | 5 | 20.19% | 2.49% |
| S27 | 5 | 19.93 | 4.25 | 5 | 0.93 | 0.14 | 5 | 32.39% | 6.91% |
| S28 | 5 | 25.82 | 2.57 | 5 | 1.20 | 0.32 | 5 | 19.03% | 1.89% |
| S29 | 4 | 83.59 | 7.43 | 4 | 2.86 | 0.23 | 4 | 43.50% | 3.87% |

S29 delivered the most (p < 0.05; ~1.6-fold more) 5-MeO-DMT to the receptor solution after 1 hour compared to S12. S12 delivered more (p < 0.05; ~1.5-fold) 5-MeO-DMT to the receptor solution compared to all other pharmaceutical compositions or formulations.

Regarding the peak flux, S29 also had the greatest (p<0.05; ~1.8-fold) peak flux between the tested pharmaceutical compositions or formulations. However, considering percent applied dose, S12 delivered more 5-MeO-DMT to the receptor solution as a cumulative percentage (p<0.05; 2-fold) and a peak flux percentage (p<0.05; 1.8-fold). It should be noted that S29 contains nearly double the (4.02% w/w) API compared to S01, S12, S26, and S28 (2.01% w/w).

In an embodiment, there is therefore provided a buccal pharmaceutical composition/formulation of 5-MeO-DMT or a pharmaceutically acceptable salt thereof, as described herein. In an embodiment, the buccal pharmaceutical composition/formulation comprises an alcohol. In an embodiment, the buccal pharmaceutical composition/formulation comprises ethanol. In an embodiment, the buccal pharmaceutical composition/formulation comprises a copolymer of poly(ethylacrylate, methyl-methacrylate, and chloro trimethyl-ammonioethyl methacrylate). In an embodiment, the buccal pharmaceutical composition/formulation comprises Eudragit. In an embodiment, the buccal pharmaceutical composition/formulation comprises Eudragit RS100. In an embodiment, the buccal pharmaceutical composition/formulation comprises Eudragit E100. In an embodiment, the buccal pharmaceutical composition/formulation comprises propylene glycol. In an embodiment, the buccal pharmaceutical composition/formulation comprises a hydrogluorocarbon. In an embodiment, the buccal pharmaceutical composition/formulation comprises 1.1, 1,2-tetrafluoroethane. In an embodiment, the buccal pharmaceutical composition/formulation comprises between 1% to 30%, 1% to 25%, 1% to 20%, 1% to 15%, 1% to 10%, 1% to 9%, 1% to 8%, 1% to 7%, 1% to 6%, 1% to 5%, 1% to 4%, 1% to 3%, 1% to 2% API. In an embodiment, the buccal pharmaceutical composition/formulation comprises one or more pharmaceutically acceptable carriers or excipients, wherein each carrier or excipient are individually present as between 1% to 50%, 1% to 40%, 1% to 30%, 1% to 25%, 1% to 20%, 1% to 15%, 1% to 10%, 1% to 9%, 1% to 8%, 1% to 7%, 1% to 6%, 1% to 5%, 1% to 4%, 1% to 3%, 1% to 2% w/w of the total buccal formulation.

### Example 31: Vaporisable Pharmaceutical Compositions or Formulations of 5-MeO-DMT

The inhalation route of administration allows for high bioavailability and low variability in bioavailability between patients and between re-administrations in the same patient, and it therefore allows for a high rate and a high reproducibility of peak experiences. A 5-MeO-DMT aerosol may be generated by volatilization of the drug by way of a vaporiser device. Such a device may comprise a hot air generator and a detachable valve balloon from which the aerosol may be inhaled by the patient. The hot air generator may generate temperatures adjustable between about 40° C. to about 210° C., with an airflow rate of about 12 litres per minute. The central part of the device may be a dosing capsule to which relevant doses of 5-MeO-DMT in an alcohol solution may be applied and which may then be applied into the filling chamber of the device, where it may be heated via the hot air. The dosing capsules may contain a small disc made of tightly packed stainless-steel wire mesh (called the drip pad or liquid pad). The bottom and the lid of the dosing capsules may have holes, allowing airflow through the dosing capsules.

To prepare for the administration, a patient may be asked to initially perform 1-2 deep inhalations with full exhalations, ending this sequence with a deep exhalation. Then, with the mouthpiece firmly held against the lips, the full and complete volume of the inhalation balloon may be inhaled in one inhalation, holding the breath for 10 (±2.5) seconds, followed by a normal exhalation. After completing the inhalation procedure, the patient may be instructed to lie down.

The thermal decomposition properties of a range of salt forms of 5-MeO-DMT has been investigated and is summarised in the report below:

| Salt | TGA Guidance |
|---|---|
| Phosphate | Decomp around 200 °C |
| Fumarate | Decomp around 200 °C |
| Oxalate | Melts 176 °C, full mass loss |
| Tartrate | Melt ~140 °C, decomp ~200 °C |
| Benzenesulfonate | Melt 76 °C, gross decomp starts 280 °C |
| Tosylate | Melt 110 °C, gross decomp starts 280 °C |
| Hydrobromide | Melt 149 °C, gross decomp starts 220 °C |
| Glycolate | Melt 95 °C, gross decomp starts 170 °C |
| Ketoglutarate | Melt 85 °C, gross decomp starts 170 °C |
| Saccharinate | Melt 80 °C, gross decomp starts 180 °C |
| Malate | Melt 100 °C, gross decomp starts 250 °C |
| Benzoate | Melt ~123 °C, decomp starts ~220 °C, full mass loss |

The benzoate and oxalate salts were the only salt forms which underwent full mass loss and within a reasonable temperature range. There is therefore provided an advantageous salt form of 5-MeO-DMT for vaporisation wherein said form is the benzoate or oxalate salt form. In an embodiment, the 5-MeO-DMT salt is in a crystalline form.

In an embodiment, there is provided a vaporisable pharmaceutical composition/formulation of 5-MeO-DMT benzoate. In an embodiment, there is provided a method of treatment comprising administration of a pharmaceutical composition/formulation of pharmaceutical composition/formulation of 5-MeO-DMT benzoate by vaporisation. In an embodiment, there is provided a pharmaceutical composition/formulation comprising 5-MeO-DMT benzoate and an alcohol. In an embodiment, the alcohol is ethanol. In an embodiment, there is provided a 5-MeO-DMT benzoate aerosol.

In an embodiment, there is provided a vaporisable pharmaceutical composition/formulation of 5-MeO-DMT oxalate. In an embodiment, there is provided a method of treatment comprising administration of a pharmaceutical composition/formulation of pharmaceutical composition/formulation of 5-MeO-DMT oxalate by vaporisation. In an embodiment, there is provided a pharmaceutical composition/formulation comprising 5-MeO-DMT oxalate and an alcohol. In an embodiment, the alcohol is ethanol. In an embodiment, there is provided a 5-MeO-DMT oxalate aerosol.

1. An active nasal delivery device comprising a pharmaceutical composition or formulation of 5-MeO-DMT, or a pharmaceutically acceptable salt thereof, and one or more pharmaceutically acceptable carriers or excipients.

2. The active nasal delivery device of claim 1, wherein the device is configured to emit a powder plume comprising 5-MeO-DMT, or a pharmaceutically acceptable salt thereof, having one or more of:
a plume geometry of:
angle: 20 to 45 degrees;
width: 25 to 55 mm;
a spray pattern of:

| Distance 40 mm: | Distance 70 mm: |
|---|---|
| Dmin(mm): 15 to 25 | Dmin(mm): 15 to 40 |
| Dmax(mm): 20 to 55 | Dmax(mm): 35 to 60 |
| Area (mm²): 390 to 900 | Area (mm²): 800-1600 |
| Area %: 2 to 15 | Area %: 4 to 20 |

a particle size distribution (at 40 mm) of:
D10=13 to 17, D50=35 to 60, D90=650 to 700, %<10 μm=<0.1, 1, 2, 3, 4, 5, 6, 7, 8, 9 or 10%; or
a particle size distribution (at 70 mm) of:
D10=13 to 17, D50=24 to 30, D90=540 to 610, %<10 μm=<0.1, 1, 2, 3, 4, 5, 6, 7, 8, 9 or 10%; or
a particle size distribution of:
D10=13 to 17, D50=22 to 27, D90=35 to 56, %<9 μm=<0.1-10%; or
% particles of equal to or less than 11.7 μm size of:
0.5 to 5%.

3. The active nasal delivery device of claim 1 or claim 2, wherein the pharmaceutical composition or formulation of 5-MeO-DMT is a spray dried or dry blended dry powder pharmaceutical composition or formulation.

4. The active nasal delivery device of any one of claims 1 to 3, wherein the pharmaceutical composition or formulation comprises below about 5% moisture content by weight of the pharmaceutical composition or formulation.

5. The active nasal delivery device of any one of claims 1 to 4, wherein at least 95% of the particles of the pharmaceutical composition or formulation are larger than 10 microns in size.

6. The active nasal delivery device of any one of claims 1 to 5, wherein the pharmaceutical composition or formulation comprises methyl cellulose, optionally a high viscosity methyl cellulose.

7. The active nasal delivery device of any one of claims 1 to 6, wherein the pharmaceutical composition or formulation comprises a low viscosity methyl cellulose, and a high viscosity methyl cellulose.

8. The active nasal delivery device of any one of claims 1 to 7, wherein the pharmaceutical composition or formulation comprises a cellulose like/based excipient; optionally HPMC, further optionally a high viscosity HPMC.

9. The active nasal delivery device of claim 8, wherein the pharmaceutical composition or formulation comprises a low viscosity HPMC and a high viscosity HPMC.

10. The active nasal delivery device of claim 9, wherein the ratio of the low viscosity HPMC to high viscosity HPMC is in the ratio of 1:10 to 10:1, optionally 1:4 to 4:1 and further optionally 1:2 to 2:1.

11. The active nasal delivery device of claim 9, wherein the ratio of the low viscosity HPMC to high viscosity HPMC is in the ratio of 50:50, 45:55, 40:60, 35:65, 30:70 or 25:75.

12. The active nasal delivery device of claim 8, wherein the high viscosity HPMC has a viscosity greater or equal to about 20, 30, 40, 50 or 60 megaPascals, optionally where the HPMC is a HPMC containing about 7.0-12.0% hydroxypropyl content, about 28.0-30.0% methoxy content, and a viscosity of about 50 mPas.

13. The active nasal delivery device of claim 8, wherein the low viscosity HPMC has a viscosity less than about 20, 15, 10, 5, 1 megaPascals, optionally where the HPMC is a HPMC containing about 7.0-12.0% hydroxypropyl content, about 28.0-30.0% methoxy content, and a viscosity of about 4.8-7.2 mPas.

14. The active nasal delivery device of any one of claims 1 to 13, wherein the pharmaceutical composition or formulation comprises a polyol, optionally the polyol is mannitol, xylitol, sorbitol, maltitol, erythritol, lactitol or isomalt, further optionally the polyol is sorbitol.

15. The active nasal delivery device of claim 14, wherein the pharmaceutical composition or formulation comprises about 1-10%, 2-5% or 3% polyol by weight, optionally about 3% sorbitol or mannitol or isomalt by weight.

16. The active nasal delivery device of any one of claims 1 to 15, wherein the pharmaceutical composition or formulation comprises a 5-MeO-DMT salt.

17. The active nasal delivery device of any one of claims 1 to 15, wherein the pharmaceutical composition or formulation comprises 5-MeO-DMT benzoate.

18. The active nasal delivery device of any one of claims 1 to 15, wherein the pharmaceutical composition or formulation comprises 5-MeO-DMT hydrochloride.

19. The active nasal delivery device of any one of claims 1 to 15, wherein the pharmaceutical composition or formulation comprises 5-MeO-DMT hydrobromide.

20. The active nasal delivery device of any one of claims 16 to 19, wherein the 5-MeO-DMT salt is amorphous.

21. The active nasal delivery device of any one of claims 16 to 19, wherein the 5-MeO-DMT salt is crystalline.

22. The active nasal delivery device of claim 21, wherein the crystalline 5-MeO-DMT salt is selected from:
a crystalline form of 5-MeO-DMT benzoate, characterised by one or more peaks in an XRPD diffractogram at 17.5, 17.7 and 21.0° 2θ±0.1°2θ as measured using an x-ray wavelength of 1.5406 Å;
a crystalline form of 5-MeO-DMT hydrochloride, characterised by one or more peaks in an XRPD diffractogram at 9.2°±0.1°, 12.2°±0.1°, 14.1°±0.1°, 15.0°±0.1°, 18.5°±0.1°, and 19.5°±0.1°2θ as measured by x-ray powder diffraction using an x-ray wavelength of 1.5406 Å; or
a crystalline form of 5-MeO-DMT hydrobromide, characterised by one or more peaks in an XRPD diffractogram at 14.6, 16.8, 20.8, 24.3, 24.9 and 27.5° 2θ±0.1°2θ as measured by x-ray powder diffraction using an x-ray wavelength of 1.5406 Å.

23. The active nasal delivery device of any one of claims 1 to 22, wherein the active nasal delivery device comprises: a dispenser outlet; an air expeller for generating a flow of air while the device is being actuated; and at least one reservoir that contains a single dose of pharmaceutical composition or formulation.

24. The active nasal delivery device of any one of claims 1 to 22, wherein the active nasal delivery device comprises: a dispenser outlet; an air expeller for generating a flow of air while the device is being actuated, said air expeller including a piston that slides in an air chamber between a rest position and a dispensing position, said air chamber including a cylindrical body in which said piston slides in airtight manner; and at least one reservoir that contains a single dose of pharmaceutical composition or formulation, said reservoir including an air inlet that is connected to said air expeller, and a pharmaceutical composition or formulation outlet that is connected to said dispenser outlet, said air inlet including a pharmaceutical composition or formulation retainer member for retaining the pharmaceutical composition or formulation in the reservoir until the pharmaceutical composition or formulation is dispensed, and said pharmaceutical composition or formulation outlet being closed by a closure element that is force fitted in the pharmaceutical composition or formulation outlet of the reservoir; said device further including a mechanical opening system that co-operates with said closure element so as to expel said closure element mechanically from a closed position while the device is being actuated, said piston of said air expeller, when in the rest position, co-operating in non-airtight manner with said air chamber, in such a manner that said air chamber is in communication with the atmosphere in the rest position, wherein said piston includes an inner lip configured to cooperate with a cylindrical surface of a cylindrical member extending inside the cylindrical body, said cylindrical surface including fluting that co-operates in non-airtight manner with said inner lip of the piston in the rest position.

25. The active nasal delivery device of claim 23 for use in a method of treating a disease or condition selected from: conditions caused by dysfunctions of the central nervous system, conditions caused by dysfunctions of the peripheral nervous system, conditions benefiting from sleep regulation (such as insomnia), conditions benefiting from analgesics (such as chronic pain), migraines, trigeminal autonomic cephalgias (such as short-lasting unilateral neuralgiform headache with conjunctival injection and tearing (SUNCT), and short-lasting neuralgiform headaches with cranial autonomic symptoms (SUNA)), conditions benefiting from neurogenesis (such as stroke, traumatic brain injury, Parkinson's dementia), conditions benefiting from anti-inflammatory treatment, depression, treatment resistant depression, anxiety, substance use disorder, addictive disorder, gambling disorder, eating disorders, obsessive-compulsive disorders, or body dysmorphic disorders, optionally the condition is SUNCT and/or SUNA, alcohol-related diseases and disorders, eating disorders, impulse control disorders, nicotine-related disorders, tobacco-related disorders, methamphetamine-related disorders, amphetamine-related disorders, cannabis-related disorders, cocaine-related disorders, hallucinogen use disorders, inhalant-related disorders, benzodiazepine abuse or dependence related disorders, opioid-related disorders, tobacco addiction, alcohol abuse and/or addiction.

26. The active nasal delivery device of claim 24 for use in a method of treating a disease or condition selected from: conditions caused by dysfunctions of the central nervous system, conditions caused by dysfunctions of the peripheral nervous system, conditions benefiting from sleep regulation (such as insomnia), conditions benefiting from analgesics (such as chronic pain), migraines, trigeminal autonomic cephalgias (such as short-lasting unilateral neuralgiform headache with conjunctival injection and tearing (SUNCT), and short-lasting neuralgiform headaches with cranial autonomic symptoms (SUNA)), conditions benefiting from neurogenesis (such as stroke, traumatic brain injury, Parkinson's dementia), conditions benefiting from anti-inflammatory treatment, depression, treatment resistant depression, anxiety, substance use disorder, addictive disorder, gambling disorder, eating disorders, obsessive-compulsive disorders, or body dysmorphic disorders, optionally the condition is SUNCT and/or SUNA, alcohol-related diseases and disorders, eating disorders, impulse control disorders, nicotine-related disorders, tobacco-related disorders, methamphetamine-related disorders, amphetamine-related disorders, cannabis-related disorders, cocaine-related disorders, hallucinogen use disorders, inhalant-related disorders, benzodiazepine abuse or dependence related disorders, opioid-related disorders, tobacco addiction, alcohol abuse and/or addiction.

27. A dry powder pharmaceutical composition or formulation, produced by spray drying, lyophilisation or hot melt extrusion, wherein the pharmaceutical composition or formulation comprises 5-MeO-DMT, or a pharmaceutically acceptable salt thereof, and one or more pharmaceutically acceptable carriers or excipients.

28. The pharmaceutical composition or formulation of claim 27, wherein the 5-MeO-DMT, or a pharmaceutically acceptable salt thereof, is in an amorphous (non-crystalline) form.

29. The pharmaceutical composition or formulation of claim 27 or 28, wherein the pharmaceutical composition or formulation is a stable free flowing pharmaceutical composition or formulation

30. The pharmaceutical composition or formulation of claim 27 or claim 28, wherein the pharmaceutical composition or formulation comprises at least about 10%, 20%, 30%, 40%, 50%, 60%, 70%, 80%, 90%, 95% or 99% by weight 5-MeO-DMT, or a pharmaceutically acceptable salt thereof.

31. The pharmaceutical composition or formulation of any one of claims 27 to 30, wherein the pharmaceutical composition or formulation exhibits an extended release profile, optionally having a residence time in the nasal cavity of at least 10, 15, 20, 25 or 30 minutes.

32. The pharmaceutical composition or formulation of any one of claims 27 to 31, wherein the pharmaceutical composition or formulation exhibits an extended release profile, wherein 80% of the 5-MeO-DMT active agent is released over a period of time of about 2 to 40, optionally 3 to 30, further optionally 4 to 15 minutes.

33. The pharmaceutical composition or formulation of any one of claims 27 to 32, wherein the pharmaceutical composition or formulation comprises a cellulose like/based excipient; optionally HPMC, further optionally a high viscosity HPMC, still further optionally a high viscosity HPMC, and wherein the pharmaceutical composition or formulation exhibits an extended release profile.

34. The pharmaceutical composition or formulation of any one of claims 27 to 33, wherein the pharmaceutical composition or formulation comprises a low viscosity HPMC and a high viscosity HPMC, and wherein the pharmaceutical composition or formulation exhibits an extended release profile.

35. The pharmaceutical composition or formulation of claim 34, wherein the ratio of the low viscosity HPMC to high viscosity HPMC is in the ratio of 1:10 to 10:1, optionally 1:4 to 4:1 and further optionally 1:2 to 2:1.

36. The pharmaceutical composition or formulation of claim 34 or 35, wherein the ratio of the low viscosity HPMC to high viscosity HPMC is in the ratio of 50:50, 45:55, 40:60, 35:65, 30:70 or 25:75 and wherein the pharmaceutical composition or formulation exhibits an extended release profile.

37. The pharmaceutical composition or formulation of any one of claims 33 to 36, wherein the high viscosity HPMC has a viscosity greater or equal to about 20, 30, 40, 50 or 60 megaPascals, optionally where the HPMC is metolose 60SH50.

38. The pharmaceutical composition or formulation of any one of claims 33 to 37, wherein the low viscosity HPMC has a viscosity less than about 20, 15, 10, 5, 1 megaPascals, optionally where the HPMC is pharmacoat 606.

39. The pharmaceutical composition or formulation of any one of claims 27 to 38, wherein the pharmaceutical composition or formulation comprises a polyol, optionally the polyol is mannitol, xylitol, sorbitol, maltitol, erythritol, lactitol or isomalt, further optionally the polyol is sorbitol.

40. The pharmaceutical composition or formulation of any one of claims 27 to 39, wherein the pharmaceutical composition or formulation comprises about 1-10%, 2-5% or 3% polyol by weight, optionally about 3% sorbitol by weight.

41. The pharmaceutical composition or formulation of any one of claims 27 to 40, wherein the pharmaceutical composition or formulation comprises 5-MeO-DMT hydrochloride, optionally the salt is in an amorphous form, further optionally a state-stable amorphous form.

42. The pharmaceutical composition or formulation of any one of claims 27 to 40, wherein the pharmaceutical composition or formulation comprises 5-MeO-DMT hydrobromide, optionally the salt is in an amorphous form, further optionally a state-stable amorphous form.

43. The pharmaceutical composition or formulation of any one of claims 27 to 40, wherein the pharmaceutical composition or formulation comprises 5-MeO-DMT benzoate, optionally the salt is in an amorphous form, further optionally a state-stable amorphous form.

44. The pharmaceutical composition or formulation of any one of claims 27 to 40, wherein the pharmaceutical composition or formulation comprises 5-MeO-DMT oxalate, optionally the salt is in an amorphous form, further optionally a state-stable amorphous form.

45. A nasal delivery device comprising the pharmaceutical composition or formulation of any one of claims 27 to 44.

46. The nasal delivery device of claim 45, wherein the device is single use.

47. The nasal delivery device of claim 46, wherein the device contains a single dose of the pharmaceutical composition or formulation.

48. The pharmaceutical composition or formulation of any one of claims 27 to 44, or the nasal delivery device of any one of claims 45 to 47, for use as a medicament.

49. The pharmaceutical composition or formulation of any one of claims 27 to 44, or the nasal delivery device of any one of claims 45 to 47, for use in a method of treatment of depression and/or alcohol use disorder.

50. The pharmaceutical composition or formulation of any one of claims 27 to 44, wherein the pharmaceutical composition or formulation is produced by spray drying and wherein following the spray drying of the pharmaceutical composition or formulation, an additional drying step is performed to condition the pharmaceutical composition or formulation and optionally wherein the drying step is performed at between 45 and 15 °C and between 85% to 65% relative humidity (RH); between 35 and 20 °C and between 80% to 70% RH; and further optionally at 25 °C and 75% RH.

51. A dry powder pharmaceutical composition or formulation, produced by a method of spray drying, wherein the pharmaceutical composition or formulation comprises about: 50% by weight 5-MeO-DMT, or a pharmaceutically acceptable salt thereof; 35% by weight HPMC 606; 12% by weight Metolose 60 SH 50; and 3% by weight of sorbitol.

52. A state-stable amorphous dry powder pharmaceutical composition or formulation comprising 5-MeO-DMT HBr and one or more pharmaceutically acceptable carriers or excipients.

53. The pharmaceutical composition or formulation of claim 52, wherein the pharmaceutical composition or formulation is a spray dried pharmaceutical composition or formulation.

54. The pharmaceutical composition or formulation of claim 52, wherein the pharmaceutical composition or formulation is a free flowing pharmaceutical composition or formulation.

55. The pharmaceutical composition or formulation of claim 52, wherein the 5-MeO-DMT HBr is non-hygroscopic.

56. The pharmaceutical composition or formulation of claim 52, wherein the pharmaceutical composition or formulation comprises below about 5% moisture content by weight of the pharmaceutical composition or formulation.

57. The pharmaceutical composition or formulation of claim 52, wherein greater than 70% (w/w) of the 5-MeO-DMT HBr in the pharmaceutical composition or formulation is in an amorphous form.

58. The pharmaceutical composition or formulation of claim 52, wherein the pharmaceutical composition or formulation comprises at least about 10%, 20%, 30%, 40%, 50%, 60%, 70%, 80%, 90%, 95% or 99% by weight 5-MeO-DMT HBr.

59. The pharmaceutical composition or formulation of claim 52, wherein at least 95% of the particles of the pharmaceutical composition or formulation are larger than 10 microns in size.

60. The pharmaceutical composition or formulation of claim 52, wherein no more than 80% of the 5-MeO-DMT is released from the pharmaceutical composition or formulation by 4 minutes in water at 37° C.

61. The pharmaceutical composition or formulation of claim 52, wherein upon administration to a nasal cavity of a subject the pharmaceutical composition or formulation exhibits a residence time, the length of time a substance is present in nasal cavity, for example along the nasal cilia and mucus layer, in the nasal cavity of at least 10, 15, 20, 25 or 30 minutes.

62. The pharmaceutical composition or formulation of claim 52, wherein the pharmaceutical composition or formulation comprises a cellulose like/based excipient; optionally HPMC, further optionally a high viscosity HPMC, still further optionally a high viscosity HPMC.

63. The pharmaceutical composition or formulation of claim 62, wherein the pharmaceutical composition or formulation comprises a low viscosity HPMC and a high viscosity HPMC.

64. The pharmaceutical composition or formulation of claim 63, wherein the ratio of the low viscosity HPMC to high viscosity HPMC is in the ratio of 1:10 to 10:1, optionally 1:4 to 4:1 and further optionally 1:2 to 2:1.

65. The pharmaceutical composition or formulation of claim 64, wherein the ratio of the low viscosity HPMC to high viscosity HPMC is in the ratio of 50:50, 45:55, 40:60, 35:65, 30:70 or 25:75.

66. The pharmaceutical composition or formulation of claim 62, wherein the high viscosity HPMC has a viscosity greater or equal to about 20, 30, 40, 50 or 60 megaPascals, optionally where the HPMC is a HPMC containing about 7.0-12.0% hydroxypropyl content, about 28.0-30.0% methoxy content, and a viscosity of about 50 mPas.

67. The pharmaceutical composition or formulation of claim 62, wherein the low viscosity HPMC has a viscosity less than about 20, 15, 10, 5, 1 megaPascals, optionally where the HPMC is a HPMC containing about 7.0-12.0% hydroxypropyl content, about 28.0-30.0% methoxy content, and a viscosity of about 4.8-7.2 mPas.

68. The pharmaceutical composition or formulation of claim 52, wherein the pharmaceutical composition or formulation comprises a polyol, optionally the polyol is mannitol, xylitol, sorbitol, maltitol, erythritol, lactitol or isomalt, further optionally the polyol is sorbitol.

69. The pharmaceutical composition or formulation of claim 68, wherein the pharmaceutical composition or formulation comprises about 1-10%, 2-5% or 3% polyol by weight, optionally about 3% sorbitol by weight.

70. A nasal delivery device comprising the pharmaceutical composition or formulation of claim 52.

71. A method of treating depression and/or alcohol use disorder in a subject in need thereof, the method comprising intranasally administering to the subject the pharmaceutical composition or formulation of claim 52 in an amount sufficient to treat the depression and/or alcohol use disorder.

72. A method of making the pharmaceutical composition or formulation of claim 52, the method comprising (i) mixing the components of the pharmaceutical composition or formulation with a liquid to form a mixture and spray drying the mixture to form a solid; and (ii) following following step (i), further drying the solid to form the pharmaceutical composition or formulation and optionally wherein the drying step is performed at between 45 and 15 °C and between 85% to 65% relative humidity (RH); between 35 and 20 °C and between 80% to 70% RH; and further optionally at 25° C. and 75% RH.

73. The method of claim 72, wherein the pharmaceutical composition or formulation comprises about: 40-60% by weight 5-MeO-DMT HBr; 30-40% by weight a HPMC containing about 7.0-12.0% hydroxypropyl content, about 28.0-30.0% methoxy content, and a viscosity of about 4.8-7.2 mPas; 7-15% by weight a HPMC containing about 7.0-12.0% hydroxypropyl content, about 28.0-30.0% methoxy content, and a viscosity of about 50 mPas; and 0-5% by weight of sorbitol.

74. A dispenser device, for dispensing the pharmaceutical composition or formulation of any one of claims 51 to 69, the dispenser device comprising: the pharmaceutical composition or formulation of any one of claims 51 to 69; a dispenser outlet; an air expeller for generating a flow of air while the device is being actuated, said air expeller including a piston that slides in an air chamber between a rest position and a dispensing position, said air chamber including a cylindrical body in which said piston slides in airtight manner; and at least one reservoir that contains a single dose of pharmaceutical composition or formulation, said reservoir including an air inlet that is connected to said air expeller, and a pharmaceutical composition or formulation outlet that is connected to said dispenser outlet, said air inlet including a pharmaceutical composition or formulation retainer member for retaining the pharmaceutical composition or formulation in the reservoir until the pharmaceutical composition or formulation is dispensed, and said pharmaceutical composition or formulation outlet being closed by a closure element that is force fitted in the pharmaceutical composition or formulation outlet of the reservoir; said device further including a mechanical opening system that co-operates with said closure element so as to expel said closure element mechanically from a closed position while the device is being actuated, said piston of said air expeller, when in the rest position, co-operating in non-airtight manner with said air chamber, in such a manner that said air chamber is in communication with the atmosphere in the rest position, wherein said piston includes an inner lip configured to cooperate with a cylindrical surface of a cylindrical member extending inside the cylindrical body, said cylindrical surface including fluting that co-operates in non-airtight manner with said inner lip of the piston in the rest position.

75. The dispenser device of claim 74 for use in a method of treating a mental health condition/disease in a subject in need thereof, the method comprising intranasally administering to the subject the pharmaceutical composition or formulation of any one of claims 51 to 69, in an amount sufficient to treat the mental health condition/disease, via the dispenser device of claim 74.

76. The dispenser device of claim 75 for use in a method of treating depression and/or alcohol use disorder in a subject in need thereof, the method comprising intranasally administering to the subject the pharmaceutical composition or formulation of any one of claims 51 to 69, in an amount sufficient to treat depression and/or alcohol use disorder, via the dispenser device of claim 74.

77. A vaporisable pharmaceutical composition or formulation of 5-MeO-DMT benzoate.

78. An aerosol comprising 5-MeO-DMT benzoate.

79. A vaporisable pharmaceutical composition or formulation of 5-MeO-DMT oxalate.

80. An aerosol comprising 5-MeO-DMT oxalate.

81. A buccal pharmaceutical composition or formulation of 5-MeO-DMT, or a pharmaceutically acceptable salt thereof, and (i) an alcohol, optionally ethanol; and (ii) a copolymer of poly(ethylacrylate, methyl-methacrylate, and chloro trimethyl-ammonioethyl methacrylate).

82. A lyophilised pharmaceutical composition or formulation comprising: (i) 5-MeO-DMT, or a pharmaceutically acceptable salt thereof; and (ii) trehalose.

83. The lyophilized pharmaceutical composition or formulation of claim 82, wherein the composition or formulation comprises 5-MeO-DMT oxalate.

84. The lyophilized pharmaceutical composition or formulation of claim 82, wherein the composition or formulation comprises 5-MeO-DMT benzoate.

85. The lyophilized pharmaceutical composition or formulation of claim 82, wherein the composition or formulation comprises 5-MeO-DMT hydrobromide.

86. The lyophilized pharmaceutical composition or formulation of any one of claims 82 to 85, wherein the pharmaceutical composition or formulation is amorphous.

87. The lyophilized pharmaceutical composition or formulation of any one of claims 82 to 86, wherein the pharmaceutical composition or formulation comprises 50% 5-MeO-DMT and 50% trehalose.

88. A lyophilised pharmaceutical composition or formulation comprising: (i) 5-MeO-DMT, or a pharmaceutically acceptable salt thereof; and (ii) lactose monohydrate.

89. The lyophilized pharmaceutical composition or formulation of claim 88, wherein the composition or formulation comprises 5-MeO-DMT oxalate.

90. The lyophilized pharmaceutical composition or formulation of claim 88, wherein the composition or formulation comprises 5-MeO-DMT benzoate.

91. The lyophilized pharmaceutical composition or formulation of claim 88, wherein the composition or formulation comprises 5-MeO-DMT hydrobromide.

92. The lyophilized pharmaceutical composition or formulation of any one of claims 88 to 91, wherein the pharmaceutical composition or formulation is amorphous.

93. The lyophilized pharmaceutical composition or formulation of any one of claims 88 to 92, lactose monohydrate.

94. A lyophilised pharmaceutical composition or formulation comprising: (i) 5-MeO-DMT, or a pharmaceutically acceptable salt thereof; and (ii) polyvinylpyrrolidone (PVP).

95. The lyophilized pharmaceutical composition or formulation of claim 94, wherein the composition or formulation comprises 5-MeO-DMT oxalate.

96. The lyophilized pharmaceutical composition or formulation of claim 94, wherein the composition or formulation comprises 5-MeO-DMT benzoate.

97. The lyophilized pharmaceutical composition or formulation of claim 94, wherein the composition or formulation comprises 5-MeO-DMT hydrobromide.

98. The lyophilized pharmaceutical composition or formulation of any one of claims 94 to 97, wherein the pharmaceutical composition or formulation is amorphous.

99. The lyophilized pharmaceutical composition or formulation of any one of claims 94 to 98, wherein the pharmaceutical composition or formulation comprises 50% 5-MeO-DMT and 50% PVP.

100. The lyophilized pharmaceutical composition or formulation of any one of claims 82 to 99, wherein the pharmaceutical composition or formulation is for use as a medicament.

101. A method of preparation of the lyophilized pharmaceutical composition or formulation of any one of claims 82 to 99.
